(19) United States
(12) Patent Application Publication
(10) Pub. No.: US 2024/0132467 A1
(43) Pub. Date: Apr. 25, 2024

(54) BENZIMIDAZOLE DERIVATIVES FOR TREATING RESPIRATORY DISEASE

(71) Applicant: TMEM16A Limited, Welwyn Garden City (GB)

(72) Inventors: Stephen COLLINGWOOD, Hayward's Heath (GB); Jonathan David Hargrave, Bath (GB); Duncan Alexander Hay, Wallingford (GB); Clive McCarthy, Wantage (GB); Thomas Beauregard Schofield, Oxford (GB); Edward Walker, Didcot (GB); Naomi Went, Didcot (GB)

(73) Assignee: TMEM16A Limited, Welwyn Garden City (GB)

(21) Appl. No.: 18/332,551

(22) Filed: Jun. 9, 2023

Related U.S. Application Data
(63) Continuation of application No. PCT/IB21/00862, filed on Dec. 10, 2021.
(60) Provisional application No. 63/124,395, filed on Dec. 11, 2020.

(51) Int. Cl. C07D 401/06; A61P 11/12; C07D 235/08; C07D 235/12; C07D 235/16; C07D 405/04; C07D 413/06

(52) U.S. Cl. CPC C07D 401/06; A61P 11/12; C07D 235/08; C07D 235/12; C07D 235/16; C07D 405/04; C07D 413/06

(57) ABSTRACT

Compounds of general formula (I): (I) wherein R¹, R², R³, R⁴, R⁵, Y and Z are as defined herein are useful for treating respiratory disease and other diseases and conditions modulated by TMEM16A.

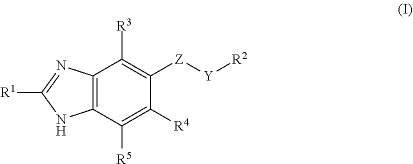

The invention relates to compounds including certain novel compounds which have activity as positive modulators of the calcium-activated chloride channel (CaCC), TMEM16A. The invention also relates to methods of preparing the compounds and pharmaceutical compositions containing them as well as to the use of these compounds in treating diseases and conditions modulated by TMEM16A, particularly respiratory diseases and conditions.

### BACKGROUND

Humans can inhale up to 12,000 L of air each day and with it comes the potential for airborne pathogens (such as bacteria, viruses and fungal spores) to enter the airways. To protect against these airborne pathogens, the lung has evolved innate defence mechanisms to minimise the potential for infection and colonisation of the airways. One such mechanism is the mucus clearance system, whereby secreted mucus is propelled up and out of the airways by the coordinated beating of cilia together with cough clearance. This ongoing ‘cleansing’ of the lung constantly removes inhaled particles and microbes thereby reducing the risk of infection.

In recent years it has become clear that the hydration of the mucus gel is critical to enable mucus clearance (Boucher 2007; Matsui et al, 1998). In a normal, healthy airway, the mucus gel is typically 97% water and 3% w/v solids under which conditions the mucus is cleared by mucociliary action. The hydration of the airway mucosa is regulated by the coordinated activity of a number of ion channels and transporters. The balance of anion (Cl⁻/HCO₃⁻) secretion mediated via the Cystic Fibrosis Transmembrane Conductance Regulator (CFTR) and the Calcium Activated Chloride Conductance (CaCC; TMEM16A) and Na⁺ absorption through the epithelial Na⁺ channel (ENaC) determine the hydration status of the airway mucosa. As ions are transported across the epithelium, water is osmotically obliged to follow and thus fluid is either secreted or absorbed.

In respiratory diseases such as chronic bronchitis and cystic fibrosis, the % solids of the mucus gel is increased as the hydration is reduced and mucus clearance is reduced (Boucher, 2007). In cystic fibrosis, where loss of function mutations in CFTR attenuates ability of the airway to secrete fluid, the % solids can be increased to 15% which is believed to contribute towards the plugging of small airways and failure of mucus clearance. Strategies to increase the hydration of the airway mucus include either the stimulation of anion and thereby fluid secretion or the inhibition of Na⁺ absorption. To this end, stimulating the activity of TMEM16A channels will increase anion secretion and therefore increase fluid accumulation in the airway mucosa, hydrate mucus and enhance mucus clearance mechanisms.

TMEM16A, also referred to as Anoctamin-1 (Ano 1), is the molecular identity of calcium- activated chloride channels (Caputo et al, 2008; Yang et al, 2008). TMEM16A channels open in response to elevation of intracellular calcium levels and allow the bidirectional flux of chloride, bicarbonate and other anions across the cell membrane. Functionally TMEM16A channels have been proposed to modulate transepithelial ion transport, gastrointestinal peristalsis, nociception and cell migration/proliferation (Pedemonte & Galietta, 2014).

TMEM16A channels are expressed by the epithelial cells of different organs including the lungs, liver, kidney, pancreas and salivary glands. In the airway epithelium TMEM16A is expressed at high levels in mucus producing goblet cells, ciliated cells and in submucosal glands. Physiologically TMEM16A is activated by stimuli which mobilise intracellular calcium, particularly purinergic agonists (ATP, UTP), which are released by the respiratory epithelium in response to cyclical shear stress caused by breathing and other mechanical stimuli such as cough. In addition to increasing anion secretion leading to enhanced hydration of the airways, activation of TMEM16A plays an important role in bicarbonate secretion. Bicarbonate secretion is reported to be an important regulator of mucus properties and in controlling airway lumen pH and hence the activity of native antimicrobials such as defensins (Pezzulo et al, 2012).

Indirect modulation of TMEM16A, via elevation of intracellular calcium, has been clinically explored e.g. denufosol (Kunzelmann & Mall, 2003). Although encouraging initial results were observed in small patient cohorts this approach did not deliver clinical benefit in larger patient cohorts (Accurso et al 2011; Kellerman et al 2008). This lack of clinical effect was ascribed to only a transient elevation in anion secretion, the result of a short half-life of denufosol on the surface of the epithelium and receptor/pathway desensitisation, and unwanted effects of elevating intracellular calcium such as increased release of mucus from goblet cells (Moss, 2013). Compounds which act directly upon TMEM16A to enhance channel opening at low levels of calcium elevation are expected to durably enhance anion secretion and mucociliary clearance in patients and improve innate defence. As TMEM16A activity is independent of CFTR function, TMEM16A positive modulators have the potential to deliver clinical benefit to all CF patients and non-CF respiratory diseases characterised by mucus congestion including chronic bronchitis and severe asthma.

TMEM16A modulation has been implicated as a therapy for dry mouth (xerostomia), resultant from salivary gland dysfunction in Sjorgen's syndrome and radiation therapy, dry eye, cholestasis and gastrointestinal motility disorders.

WO 2019/145726 relates to compounds which are positive modulators of TMEM16A and which are therefore of use in the treatment of diseases and conditions in which modulation of TMEM16A plays a role, particularly respiratory diseases and conditions. The present inventors have developed further compounds which are positive modulators of TMEM16A.

### SUMMARY OF THE INVENTION

In a first aspect of the present invention there is provided a compound of general formula (I) including all tautomeric forms all enantiomers and isotopic variants and salts and solvates thereof:

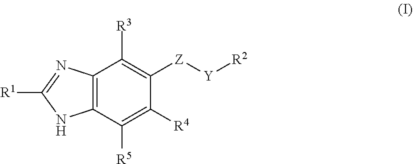

wherein:

R¹ is

i. [CH(R⁷)]ₙ—N(R⁸—C(O)OR⁹;
n is 1 or 2;
each R⁷ is independently H, phenyl or C₁₋₃ alkyl optionally substituted with one or more substituents selected from OH and OCH₃;
R⁸ is H, C₁₋₃ alkyl optionally substituted with one or more substituents selected from OH and methoxy;
R⁹ is C₂₋₄ alkyl; or

ii. CH(R¹¹)(R¹²);
R¹¹ is H, OH, CH₃, CH₂OH or a group which combines with a substituent on R¹² as defined below;
R¹² is selected from cyclohexyl optionally substituted with one or more substituents selected from OH and methoxy; and phenyl or 5- or 6-membered heteroaryl wherein said phenyl or heteroaryl is optionally substituted with one or more substituents selected from OH, methoxy, methyl, fluoro, chloro and a substituent which, together with R¹¹ and the atoms to which it is attached, forms a 5- or 6-membered oxygen-containing heterocyclic ring fused to the phenyl or heteroaryl group R¹²; or

iii. C₂₋₆ alkyl optionally substituted with OR¹⁵;
R¹⁵ is methyl or ethyl; or

iv. 6- to 10-membered aryl or 5- to 10-membered heteroaryl, either or which is optionally substituted with one or more substituents selected from fluoro, chloro, OH or methoxy;

Z is selected from —NH—C(O)— and —C(O)—NH—;

Y is selected from a bond, —CH₂— and —CH(CH₃)—; or Y combines with R² as defined below; and

R² is selected from:

a 3- to 10-membered carbocyclic ring system or a 6- to 10-membered aryl or 5- to 10-membered heteroaryl ring system, wherein the aryl, heteroaryl or carbocyclic ring system is optionally substituted with one or more substituents selected from fluoro; chloro; CN; nitro; OH; C₁₋₆ alkyl optionally substituted with one or more substituents selected from halo, OH and CN; O(C₁₋₆ alkyl) optionally substituted with one or more substituents selected from halo, OH and CN; and CH₂NH—C(O)O—C₁₋₆ alkyl optionally substituted with one or more substituents selected from halo and OH; or

Y and R² together form an unsubstituted C₃₋₈ alkyl group or a group CH₂—C(Ru)(R¹⁸)—CH₂—N(R¹⁹)R²⁰;
wherein each of R¹⁷, R¹⁸ and R¹⁹ is independently H or C₁₋₄ alkyl; and
R²⁰ is C₁₋₄ alkyl or C₁₋₄ haloalkyl;

R³, R⁴ and R⁵ are each independently either H or F;

provided that:
A. when R¹ is CH(R¹¹)(R¹²); where R¹¹ is H or methyl and R¹² is phenyl which is unsubstituted or substituted with 1 or 2 substituents, wherein the substituents are selected from halo and methoxy:
   i. R² is not phenyl or heteroaryl, wherein said phenyl or heteroaryl is optionally substituted with 1 or 2 substituents selected from halo, C₁₋₄ alkyl, C₁₋₄ alkoxy and a 5-membered heteroaryl ring; and
B. when R¹ is CH(R¹¹)(R¹²); where R¹² is phenyl and R¹¹ together with a substituent on R¹² and the atoms to which they are attached combine to form a 5- or 6-membered ring fused to the phenyl ring R¹², wherein the 5- or 6-membered ring is optionally substituted with C₁₋₃ alkyl:
   i. R² is not phenyl or heteroaryl, wherein said phenyl or heteroaryl is optionally substituted with 1, 2 or 3 substituents, wherein the substituents are selected from halo, C₁₋₄ alkyl, C₁₋₄ haloalkyl and C₁₋₄ alkoxy; and
   ii. Y and R² do not combine to form C₃₋₁₀ alkyl;
C. when R¹ is CH(R¹¹)(R¹²); where R¹¹ is H and R¹² is cyclohexyl:
   R² is not phenyl optionally substituted with 1, 2 or 3 substituents, wherein the substituents are selected from halo, methyl, methoxy; unsubstituted 5- to 8 membered heteroaryl.
      Optionally, the compound of general formula (I) is selected from:
      N-(2-Benzyl-1H-benzimidazol-5-yl)-2-cyclohexyl-acetamide (Compound 1.1);
      2-Benzyl-N-(cyclohexylmethyl)-1H-benzimidazole-5-carboxamide (Compound 1.2);
      N-(1-Adamantylmethyl)-2-benzyl-1H-benzimidazole-5-carboxamide (Compound 1.2.1);
      2-Benzyl-N-[(1-methylcyclopentyl)methyl]-1H-benzimidazole-5-carboxamide (Compound 1.2.2);
      2-Benzyl-N-[(1R)-1-cyclohexylethyl]-1H-benzimidazole-5-carboxamide (Compound 1.2.3);
      N-(Cycloheptylmethyl)-2-(1,1-dimethylpropyl)-3H-benzimidazole-5-carboxamide Compound 1.3);
      N-(Cycloheptylme thyl)-2-[(1-hydroxycyclohexyl)methyl]-1H-benzimidazole -5-carboxamide (Compound 1.3.1);
      N-(Cycloheptylmethyl)-2-(2-hydroxy-1-phenyl-ethyl)-1H-benzimidazole-5-carboxamide (Compound 1.3.2);
      N-(Cyclohexylmethyl)-2-[(3-hydroxyphenyl)methyl]-3H-benzimidazole-5-carboxamide (Compound 1.4);
      2-(1-Adamantyl)-N[2-[(3-hydroxyphenyl)methyl]-1H-benzimidazol-5-yl]acetamide (Compound 1.5);
      N(2-[(3-hydroxyphenyl)methyl]-1H-benzimidazol-5-yl1-2-(1-methylcyclohexyl) acetamide (Compound 1.5.1);
      2-Cycloheptyl-N-[2-[(3-hydroxyphenyl)methyl]-1H-benzimidazol-5-yl]acetamide (Compound 1.5.2);
      2-Cyclohexyl-N[2-[(3-hydroxyphenyl)methyl]-1H-benzimidazol-5-yl]acetamide (Compound 1.5.3);
      2-(1-Adamantyl)-N-(2-benzyl-1H-benzimidazol-5-ypacetamide (Compound 1.5.4);
      N-(2-Benzyl-1H-benzimidazol-5-yl)-2-(2-hydroxy-2-adamantypacetamide (Compound 1.6);

### 2-(2-Adamantyl)-N[2-[(3-methoxyphenyOmethyl]-1H-benzimidazol-5-yl]acetamide (Compound 1.7);

2-(2-Adamantyl)-N[2-[(1 S)-1-phenylethyl]-1H-benzimidazol-5-yl]acetamide (Compound 1.7.1);
2-(2-Adamantyl)-N[2-[(1R)-1-phenylethyl]-1H-benzimidazol-5-yl]acetamide (Compound 1.7.2);
tert-Butyl N-[[5-[[2-(1-adamantypacetyl]amino1-1H-benzimidazol-2-yl]methyl]carbamate (Compound 1.7.3);
2-(1-Adamantyl)-N[2-[(2-methoxy-3-pyridyl)methyl]-1H-benzimidazol-5-yl]acetamide (Compound 1.7.4);
2-(2-Adamantyl)-N[2-[(3-hydroxyphenyl)methyl]-1H-benzimidazol-5-yl]acetamide (Compound 1.8);
2-te rt-Butyl-N-R5-chlor o-2-hydroxy -phenyl)methyl]-1H-benzimidazole-5-carboxamide (Compound 1.9);
tert-Butyl N[[5-(cycloheptylmethylcarbamoyl)-1H-benzimidazol-2-yl]methyl] carbamate (Compound 1.10);
2-Benzyl-N-[(1-methylcyclohexyl)methyll-1H-benzimidazole-5-carboxamide (Compound 1.10.1);
2-Benzyl-N-(cyclooctylmethyl)-1H-benzimidazole-5-carboxamide (Compound 1.10.2);
tert-Butyl N-[[1-2-[(2-benzyl-1H-benzimidazol-5-yl)amino]-2-oxo-ethyl]cyclohexyl]methyl]carbamate (Compound 2.1);
N-(2-Benzyl-1H-benzimidazol-5-yl)-2-(4,4-difluorocyclohexypacetamide (Compound 2.1.1);
tert-Butyl N-[1-[5-[[2-(2-adamantypacetyl]amino]-1H-benzimidazol-2-yl1-2-methoxy-ethylicarbamate (Compound 2.2);
tert-Butyl N-[(R)-[1-[5-[[2-(2-adamantyl)acetyl]amino1-1H-benzimidazol-2-yl1-phenyl-methyl]-N-methyl-carbamate Compound 2.2.1;
tert-Butyl N-[(S)-[1-[5-[[2-(2-adamantyl)acetyl]amino1-1H-benzimidazol-2-yl1-phenyl-methyl]-N-methyl-carbamate (Compound 2.2.2);
tert-Butyl N[[6-[[2-adamantypacetyl]amino1-1H-benzimidazol-2-yl]methyl1-N-ethyl-carbamate (Compound 2.2.3);
tert-Butyl N-[[5-[[2-(2-adamantypacetyl]amino1-1H-benzimidazol-2-yl]methyl1-N-(2-methoxyethyl)carbamate (Compound 2.2.4);
2-(2-Adamantyl)-N[2-(2-methoxyethyl)-3H-benzimidazol-5-yl]acetamide (Compound 2.2.5);
2-(2-Adamantyl)-N[2-(3-methoxypropyl)-1H-benzimidazol-5-yl]acetamide (Compound 2.2.6);
tert-Butyl N-[[5-[[2-(2-adamantypacetyl]amino1-1H-benzimidazol-2-yl]methyl1-N-methyl-carbamate (Compound 2.3);
N-(Cycloheptylmethyl)-2-(2,3-dihydrobenzofuran-3-yl)-1H-benzimidazole-5-carboxamide (Compound
2.4);
2-(2-adamantyl)-N[2-[hydroxy(phenyl)methyl]-1H-benzimidazol-5-yl]acetamide (Compound 2.5);
2-Cyclohexyl-N-(2-phenyl-1H-benzimidazol-5-yOacetamide (Compound 3.1);
N-(2-Benzyl-1H-benzimidazol-5-yl)adamantane-1-carboxamide (Compound 3.2);
N-(Cycloheptylmethyl)-7-fluoro-2-[(3-hydroxyphenyl)methyl]-1H-benzimidazole-5-carboxamide (Compound 3.3);
N-(Cycloheptylmethyl)-6-fluoro-2-[(3-hydroxyphenyl)methyl]-1H-benzimidazole-5-carboxamide (Compound 3.3.1);
N-(Cycloheptylmethyl)-4-fluoro-2-[(3-hydroxyphenyl)methyl]-1H-benzimidazole-5-carboxamide (Compound 3.3.2);
tert-Butyl N-[2-[5-[[2-(2-adamantypacetyl]amino]-1H-benzimidazol-2-yllethyllcarbamate (Compound 3.4);
2-(2-Adamantyl)-N[2-[(3,5-dimethylisoxazol-4-yl)methyl]-1H-benzimidazol-5-yl]acetamide (Compound 3.4.1);
2-Benzyl-N-(2,2-dimethylpropyl)-1H-benzimidazole-5-carboxamide (Compound 3.5);
2-Benzyl-N-(1,1,2,2-tetramethylpropyl)-1H-benzimidazole-5-carboxamide (Compound 3.5.1);
N-(cycloheptylmethyl)-2-[(5-fluoro-2-methoxy-phenyl)methyl]-1H-benzimidazole-5-carboxamide;
N-(cycloheptylmethyl)-2-[(3-fluoro-2-methoxy-phenyl)methyl]-1H-benzimidazole-5-carboxamide; and their enantiomers, salts and solvates.

Compounds of general formula (I) are modulators of TMEM16A and are therefore useful for the treatment or prophylaxis of diseases and conditions affected by the modulation of TMEM16A.

### DESCRIPTION OF THE DRAWINGS

FIG. 1 is an example trace from a whole-cell patch clamp (Qpatch) TMEM16A potentiator assay as used in the Biological Example and illustrates the methodology used in the assay.

### DETAILED DESCRIPTION OF THE INVENTION

Throughout the specification and the claims which follow, unless the context requires otherwise, the word ‘comprise’, and variations such as ‘comprises’ and ‘comprising’, will be understood to imply the inclusion of a stated integer, step, group of integers or group of steps but not to the exclusion of any other integer, step, group of integers or group of steps.

In the present specification, references to “pharmaceutical use” refer to use for administration to a human or an animal, in particular a human or a mammal, for example a domesticated or livestock mammal, for the treatment or prophylaxis of a disease or medical condition. The term “pharmaceutical composition” refers to a composition which is suitable for pharmaceutical use and “pharmaceutically acceptable” refers to an agent which is suitable for use in a pharmaceutical composition. Other similar terms should be construed accordingly.

In the present specification, the term “C₁₋₆” alkyl refers to a straight or branched fully saturated hydrocarbon group having from 1 to 6 carbon atoms. The term encompasses methyl, ethyl, n-propyl, isopropyl, n-butyl, isobutyl and t-butyl. Other alkyl groups, for example C₁₋₁₀ alkyl are as defined above but contain different numbers of carbon atoms.

The terms “carbocyclic” and “carbocyclyl” refer to a non-aromatic hydrocarbon ring system containing from 3 to 10 ring carbon atoms, unless otherwise indicated, and optionally one or more double bond. The carbocyclic group may be a single ring or may contain two or three rings which may be fused or bridged, where carbon atoms in a bridge are included in the number of ring carbon atoms. Examples include cyclopropyl, cyclobutyl, cyclopentyl, cyclohexyl, cyclopentenyl and cyclohexenyl as well as bridged systems such as bicyclo[1.1.1]pentyl, bicyclo-[2.2.1]heptyl, bicyclo-[2.2.2loctyl and adamantyl.

In the context of the present specification, the terms “heterocyclic” and “heterocyclyl” refer to a non-aromatic ring system containing 3 to 10 ring atoms, unless otherwise indicated, including at least one heteroatom selected from N, O and S. The heterocyclic group may be a single ring or may contain two or three rings which may be fused or bridged, where bridge atoms are included in the number of ring atoms. Examples include tetrahydrofuranyl, tetrahydropyranyl, pyrrolidinyl, piperidinyl, morpholinyl, piperazinyl and thiomorpholinyl, as well as fused systems such as cyclopropyl-fused pyrrolidine. References to an oxygen containing heterocyclic ring include both rings in which the only heteroatom is oxygen, for example tetrahydrofuran and tetrahydropyran and also rings in which an additional heteroatom selected from N and S is present, or example morpholine.

The terms “aryl” and “aromatic” in the context of the present specification refer to a ring system with aromatic character having from 5 to 14 ring carbon atoms, unless otherwise indicated, and containing up to three rings. Where an aryl group contains more than one ring, not all rings must be fully aromatic in character. Examples of aromatic moieties are benzene, naphthalene, fluorene, tetrahydronaphthalene, indane and indene.

The terms “heteroaryl” and “heteroaromatic” in the context of the specification refer to a ring system with aromatic character having from 5 to 14 ring atoms, unless otherwise indicated, at least one of which is a heteroatom selected from N, 0 and S, and containing up to three rings. Where a heteroaryl group contains more than one ring, not all rings must be aromatic in character. Examples of heteroaryl groups include pyridine, pyrimidine, indole, indazole, thiophene, benzothiophene, benzoxazole, benzofuran, dihydrobenzofuran, tetrahydrobenzofuran, benzimidazole, benzimidazoline, quinoline and indolene.

The term “halogen” refers to fluorine, chlorine, bromine or iodine and the term “halo” to fluoro, chloro, bromo or iodo groups. Similarly, “halide” refers to fluoride, chloride, bromide or iodide.

The term “C₁₋₆ haloalkyl” as used herein refers to a C₁₋₆ alkyl group as defined above in which one or more of the hydrogen atoms is replaced by a halo group. Any number of hydrogen atoms may be replaced, up to perhalo substitution. Examples include trifluoromethyl, chloroethyl and 1,1-difluoroethyl. A fluoroalkyl group is a haloalkyl group in which halo is fluoro.

The term “isotopic variant” refers to isotopically-labelled compounds which are identical to those recited in formula (I) but for the fact that one or more atoms are replaced by an atom having an atomic mass or mass number different from the atomic mass or mass number most commonly found in nature, or in which the proportion of an atom having an atomic mass or mass number found less commonly in nature has been increased (the latter concept being referred to as “isotopic enrichment”). Examples of isotopes that can be incorporated into compounds of the invention include isotopes of hydrogen, carbon, nitrogen, oxygen, fluorine, iodine and chlorine such as 2H (deuterium), 3H, 11C, 13C, 14C, 18F, 1231 or 1251 (e.g. 3H, 11C, 14C, 18F, 1231 or 1251), which may be naturally occurring or non-naturally occurring isotopes.

The compound of general formula (I) may be also be in the tautomeric form:

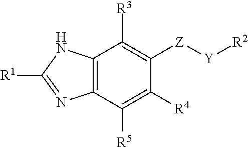

In some compounds of the present invention, R¹ is [CH(R⁷)]ₙ—N(R⁸—C(O)OR⁹, wherein n, R⁷, R⁸ and R⁹ are as defined above for general formula (I).

In some compounds of this type, n is 1 and in other such compounds, n is 2.

More suitably in such compounds, R⁷ is selected from H, phenyl, methyl, CH₂OH and CH₂OCH₃, still more suitably H, methyl, phenyl and CH₂OCH₃.

R⁸ is more suitably selected from H, methyl optionally substituted with methoxy and ethyl optionally substituted with methoxy.

R⁹ is more suitably selected from C₃₋₄ alkyl, especially n-butyl, i-butyl and t-butyl, particularly t-butyl.

In some compounds in which R¹ is [CH(R⁷)]ₙ—N(R⁸—C(O)OR⁹, wherein n is 1, R⁷ and R⁸ are not both H. For example, in some cases, R⁷ is H and R⁸ is C₁₋₃ alkyl optionally substituted with one or more substituents selected from OH and methoxy, especially methyl or ethyl. In other cases, R⁷ is phenyl or C₁₋₃ alkyl optionally substituted with one or more substituents selected from OH and OCH₃, especially phenyl, methyl or CH₂OCH₃, and R⁸ is H. In still other cases, R⁷ is phenyl or C₁₋₃ alkyl optionally substituted with one or more substituents selected from OH and methoxy and R⁸ is C₁₋₃ alkyl optionally substituted with one or more substituents selected from OH and methoxy; for example R⁷ is CH₂OCH₃ or phenyl and R⁸ is methyl or ethyl.

In some compounds of general formula (I), R¹ is CH(R¹¹) (R¹²) wherein R¹¹ and R¹² are as defined above for general formula (I).

In some suitable compounds of this type, R¹² is cyclohexyl optionally substituted with OH.

In other such compounds, R¹² is phenyl, pyridyl or oxazolyl, any of which is optionally substituted with one or more substituents selected from OH, methoxy, fluoro and chloro.

In some cases, for example, R¹² is phenyl optionally substituted at the 2-position with OH or methoxy and optionally having one or two further substituents, preferably one further substituent selected from fluoro and chloro. Examples of such R¹² groups include phenyl 2-hydroxyphenyl, 3-hydroxyphenyl, 4-hydroxyphenyl, 2-methoxyphenyl, 3-methoxyphenyl, 4-methoxyphenyl, 3-fluoro-2-methoxyphenyl, 4-fluoro-2-methoxyphenyl, 5-fluoro-2-methoxyphenyl and 5-chloro-2-methoxyphenyl.

In other cases, R¹² is a pyridyl group optionally substituted with OH or methoxy, for example 2-methoxy-pyridin-3-yl, or an oxazolyl group optionally substituted with one or two methyl groups, especially a dimethyloxazolyl group.

Alternatively, R¹² is phenyl having a substituent which, together with R¹¹ and the atoms to which it is attached, forms a 5- or 6-membered oxygen-containing heterocyclic ring fused to the phenyl group R¹².

The phenyl group R¹² may also contain other substituents as set out above. Suitably, the R¹² substituent which combines with R¹¹ is at a position on the phenyl group R¹² adjacent to the position at which the phenyl group R¹² is linked to CH(R¹¹) and the combined substituent is a 2- or 3-membered hydrocarbon chain in which a CH₂ moiety is optionally replaced with —O—. For example, R¹¹ and a substituent on R¹² may combine to form a group —O—CH₂—, —CH₂—O—, —O—CH₂—CH₂—, —CH₂—CH₂—O—. One example of this type of combined R¹¹ and R¹² group is 2,3-dihydrobenzofuran-3-yl.

In some compounds of general formula (I), R¹ is C₂₋₆ alkyl optionally substituted with OR¹⁵, wherein R¹⁵ is as defined in general formula (I). In some more suitable compounds of this type, R¹ is unsubstituted C₃₋₆ alkyl, especially a branched unsubstituted C₃₋₆ alkyl and more particularly a branched unsubstituted C₄₋₆ alkyl group. In other more suitable compounds of this type, R¹ is methyl or ethyl substituted with OR¹⁵, especially with methoxy.

In still other compounds of general formula (I), R¹ is 6- to 10-membered aryl or 5- to 10-membered heteroaryl, either or which is optionally substituted with one or more substituents selected from fluoro, chloro, OH or methoxy. More suitably, R¹ is phenyl or a 5- or 6-membered heteroaryl group, suitably a nitrogen- or oxygen-containing heteroaryl group. The phenyl or heteroaryl group may optionally be substituted as defined above but is more suitably unsubstituted. Unsubstituted phenyl is an example of an R¹ group of this type.

In some compounds of general formula (I), R² is a 3- to 10-membered carbocyclic ring system optionally substituted as defined above.

In some compounds of this type, R² is a bridged carbocyclic ring system such as bicyclo[1.1.1]pentanyl, bicyclo[2.1.1]hexanyl, bicyclo42.2.11heptanyl, bicyclo42.2.21octanyl or adamantyl, especially bicyclo-[2.2.1]heptanyl or adamantyl. Compounds in which R² is adamantyl are particularly suitable. In some cases, when R² is a bridged carbocyclic ring system, it is unsubstituted. Alternatively, a bridged carbocyclic ring system R² may be substituted, for example with OH. An example of such an R² group is adamantyl substituted with OH.

In other compounds of this type, R² is a carbocyclic ring system, particularly a 5- to 8-membered carbocyclic ring system selected from cyclopentyl, cyclohexyl, cycloheptyl and cyclooctyl, any of which may be unsubstituted or substituted as defined above. More suitable substituents for such R² groups include OH, fluoro, C₁₋₆ alkyl, O(C₁₋₆ alkyl), and NH—C(O)O—C₁₋₆ alkyl, especially OH, C₁₋₄, alkyl, O(C₁₋₄ alkyl) and NH—C(O)O—C₁₋₄ alkyl, and still more suitably C₁₋₄ alkyl (especially methyl) fluoro and NH—C(O)O—C₁₋₄ alkyl.

In other more suitable compounds of this type, R² is an unsubstituted cyclopentyl, cyclohexyl or cycloheptyl ring, especially unsubstituted cyclohexyl or cycloheptyl.

In other compounds of general formula (I), R² is a 6- to 10-membered aryl or 5- to 10-membered heteroaryl ring system, optionally substituted as defined above. More suitably in this case, R² is phenyl or a 5- or 6-membered heteroaryl ring optionally substituted with one or more substituents selected from fluoro, chloro, OH, C₁₋₆ alkyl optionally substituted with one or more substituents selected from OH and halo, O(C₁₋₆ alkyl) and O(C₁₋₆ haloalkyl), still more suitably fluoro, chloro, OH, C₁₋₄ alkyl, C₁₋₄ alkyl substituted with OH and O(C₁₋₄ alkyl); and especially fluoro, chloro, OH, C₁₋₄ alkyl, C₁₋₄ alkyl substituted with OH and methoxy. In particular, R² is phenyl substituted with OH at the 2-position and optionally with a further substituent selected from fluoro and chloro.

In some compounds of general formula (I), Y and R² together form an unsubstituted C₃₋₈ alkyl group, more suitably a C₅₋₈ alkyl group.

In some compounds of general formula (I), Y and R² together form a group CH₂—C(R¹⁷)(R¹⁸)—CH₂—N(R¹⁹)R²⁰;
wherein each of R¹⁷, R¹⁸, R¹⁹ and R²⁰ is as defined above.

More suitably, each of R¹⁷, R¹⁸ and R¹⁹ is independently H or methyl and R²⁰ is C₁₋₄ haloalkyl. Still more suitably, each of R¹⁷ and R¹⁸ is independently H or methyl, R¹⁹ is H and R²⁰ is C₁₋₄ haloalkyl.

In some particularly preferred compounds of general formula (I), R² is unsubstituted cyclohexyl and R¹ is CH(R¹¹)(R¹²), where R¹¹ and R¹² are as defined above. More particularly, R¹¹ is as defined above and R¹² is phenyl optionally substituted with OH or methoxy. Compounds where R² is unsubstituted cyclohexyl and R¹ is unsubstituted benzyl are particularly suitable, in particular N-(2-benzyl-1H-benzimidazol-5-yl)-2-cyclohexyl-acetamide; 2-benzyl-N-(cyclohexylmethyl)-1H-benzimidazole-5-carboxamide and salts and solvates thereof.

In some compounds of general formula (I), R³, R⁴ and R⁵ are all H.

In some compounds of general formula (I), one of R³, R⁴ and R⁵ is halo and the others are H. In certain compounds R³ is halo and R⁴ and R⁵ are H. In certain compounds R⁴ is halo and R³ and R⁵ are H. In certain compounds R⁵ is halo and R³ and R⁴ are H.

In other compounds of general formula (I), one or more of R³, R⁴ and R⁵ is F. For example:

R³ is F and R⁴ and R⁵ are H; or

R⁴ is F and R³ and R⁵ are H; or

R⁵ is F and R³ and R⁴ are H.

In some compounds of general formula (I), Z is -NH—C(O)—.

In some compounds of general formula (I), Z is —C(O)NH—.

In some compounds of general formula (I), Y is a bond.

In some compounds of general formula (I), Y is —CH₂—.

In some compounds of general formula (I), Y is —CH(CH₃)—.

In one embodiment, the compound of formula (I) is a compound of formula (IA) including all tautomeric forms all enantiomers and isotopic variants and salts and solvates thereof:

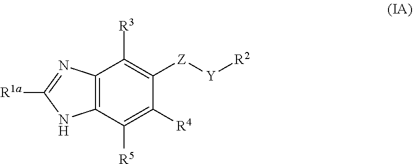

wherein R², R³, R⁴, R⁵, Y and Z are as defined for general formula (I) and:

R^{1a} is

i. [CH(R^{7a})]ₙ—N(R^{8a})—C(O)ORR^{9a};
n is 1 or 2;
each R^{7a} is independently H, phenyl or C₁₋₃ alkyl optionally substituted with one or more substituents selected from OH and OCH₃;
R^{8a} is H, C₁₋₃ alkyl optionally substituted with one or more substituents selected from OH and methoxy;
provided that when n is 1, R^{7a} and R^{8a} are not both H;
R^{9a} is C₂₋₄ alkyl; or

ii. CH(R^{11a})(R^{12a});
Rlla is H, OH, CH₃, CH₂OH or a group which combines with a substituent on R¹² a as defined below;
R¹² a is selected from phenyl or 5- or 6-membered heteroaryl, wherein said phenyl or heteroaryl is optionally substituted with one or more substituents selected from OH, methoxy, methyl, fluoro, chloro and a substituent which, together with R^{11a} and the atoms to which it is attached, forms a 5- or 6-membered oxygen-containing heterocyclic ring fused to the phenyl or heteroaryl group R^{12a}; provided that when R^{12a} is phenyl or 6-membered heteroaryl optionally substituted with one or more substituents selected from OH, methoxy, methyl, fluoro or chloro, R^{11a} is not H; or

iii. methyl, ethyl or n-propyl substituted with OR^{15a};
R^{15a} is methyl or ethyl; or

iv. 6- to 10-membered aryl or 5- to 10-membered heteroaryl, either or which is optionally substituted with one or more substituents selected from fluoro, chloro, OH or methoxy; provided that:
A. when R¹ a is CH(R^{11a})(R^{12a}); where R^{11a} is H or methyl and R^{12a} is phenyl which is unsubstituted or substituted with 1 or 2 substituents, wherein the substituents are selected from halo and methoxy:
   i. R² is not phenyl or heteroaryl, wherein said phenyl or heteroaryl is optionally substituted with 1 or 2 substituents selected from halo, C₁₋₄ alkyl, C₁₋₄ alkoxy and a 5-membered heteroaryl ring; and
B. when R¹ a is CH(R¹¹ a)(R) where R^{12 a} is phenyl and R ll a together with a substituent on
R^{12a} and the atoms to which they are attached combine to form a 5- or 6-membered ring fused to the phenyl ring R^{12a}, wherein the 5- or 6-membered ring is optionally substituted with C₁₋₃ alkyl:
   i. R^{12a} is not phenyl or heteroaryl, wherein said phenyl or heteroaryl is optionally substituted with 1, 2 or 3 substituents, wherein the substituents are selected from halo, C₁₋₄ alkyl, C₁₋₄ haloalkyl and C₁₋₄ alkoxy; and
   ii. Y and R² do not combine to form C₃₋₁₀ alkyl.

In some compounds of general formula (IA), R^{1a} is [CH(R^{7a})]ₙ—N(R^{8a})—C(O)OR^{9a}, wherein n, R^{7a}, R^{8a} and R^{9a} are as defined above for general formula (IA).

In some compounds of this type, n is 1 and in other such compounds, n is 2.

More suitably in such compounds, R^{7a} is selected from H, phenyl, methyl, CH₂OH and CH₂OCH₃, still more suitably H, methyl, phenyl and CH₂OCH₃.

R^{8a} is more suitably selected from H, methyl optionally substituted with methoxy and ethyl optionally substituted with methoxy.

R^{9a} is more suitably selected from C₃₋₄ alkyl, especially n-butyl, i-butyl and t-butyl, particularly t-butyl.

In compounds of general formula (IA), wherein R¹ is [CH(R⁷)]ₙ—N(R⁸—C(O)OR⁹, and n is 1, R^{7a} and R^{8a} are not both H. In some cases, R^{7a} is H and R^{8a} is C₁₋₃ alkyl optionally substituted with one or more substituents selected from OH and methoxy, especially methyl or ethyl. In other cases, R^{7a} is phenyl or C₁₋₃ alkyl optionally substituted with one or more substituents selected from OH and OCH₃, especially phenyl, methyl or CH₂OCH₃, and R^{8a} is H. In still other cases, R^{7a} is phenyl or C₁₋₃ alkyl optionally substituted with one or more substituents selected from OH and methoxy and R^{8a} is C₁₋₃ alkyl optionally substituted with one or more substituents selected from OH and methoxy; for example R^{7a} is CH₂OCH₃ or phenyl and R^{8a} is methyl or ethyl.

In some compounds of general formula (IA), R^{1a} is CH(R^{11a})(R^{12a}), wherein R^{11a} and R^{12a} are as defined above for general formula (IA).

In some such compounds, R^{12a} is phenyl or a 6-membered heteroaryl group such as pyridyl either of which is optionally substituted with one or more substituents selected from OH, methoxy, fluoro and chloro; and R^{11a} is OH, CH₃ or CH₃OH.

For example, R^{12a} is phenyl optionally substituted at the 2-position with OH or methoxy and optionally having one or two further substituents, preferably one further substituent selected from fluoro and chloro. Examples of such R^{12a} groups include phenyl 2-hydroxyphenyl, 3-hydroxyphenyl, 4-hydroxyphenyl, 2-methoxyphenyl, 3-methoxyphenyl, 4-methoxyphenyl, 3-fluoro-2-methoxyphenyl, 4-fluoro-2-methoxyphenyl, 5-fluoro-2-methoxyphenyl and 5-chloro-2-methoxyphenyl.

In other such compounds, R^{12a} is a pyridyl group optionally substituted with OH or methoxy, for example 2-methoxy-pyri din-3-yl.

In other compounds of general formula (IA) wherein R^{1a} is CH(R^{11a})(R^{12a}), R^{12a} is a 5-membered heteroaryl group such as oxazolyl, optionally substituted with one or two methyl groups, especially a dimethyloxazolyl group. In this case R^{11a} may be H, OH, CH₃ or CH₃OH.

In still other compounds of general formula (IA) wherein R^{1a} is CH(Rila)(R^{12a}), R12a i s phenyl having a substituent which, together with R^{11a} and the atoms to which it is attached, forms a 5- or 6-membered oxygen-containing heterocyclic ring fused to the phenyl group R^{12a}. The phenyl group R^{12a} may also contain other substituents as set out above. Suitably, the R^{12a} substituent which combines with R^{11a} is at a position on the phenyl group R^{12a} adjacent to the position at which the phenyl group R^{12a} is linked to CH(R^{11a}) and the combined substituent is a 2- or 3-membered hydrocarbon chain in which a CH₂ moiety is optionally replaced with —O—. For example, R^{11a} and a substituent on R^{12a} may combine to form a group —O—CH₂—, —CH₂—O—, —O—CH₂—CH₂—, —CH₂—CH₂—O—. One example of this type of combined R¹¹ and R¹² group is 2,3-dihydrobenzo furan-3-yl .

In some compounds of general formula (IA), R^{1a} is methyl, ethyl or n-propyl, especially ethyl or n-propyl, substituted with OR¹⁵, especially with methoxy.

In still other compounds of general formula (IA), R^{1a} is 6- to 10-membered aryl or 5- to 10-membered heteroaryl, either or which is optionally substituted with one or more substituents selected from fluoro, chloro, OH or methoxy. More suitably, R^{1a} is phenyl or a 5- or 6-membered heteroaryl group, suitably a nitrogen- or oxygen-containing heteroaryl group. The phenyl or heteroaryl group may optionally be substituted as defined above but is more suitably unsubstituted. Unsubstituted phenyl is an example of an Rla group of this type.

In compounds of general formula (IA), more suitable values for R², R³, R⁴, R⁵, Y and Z are as defined above for compounds of general formula (I).

In a further embodiment, the compound of general formula (I) is a compound of general formula (TB) including all tautomeric forms all enantiomers and isotopic variants and salts and solvates thereof:

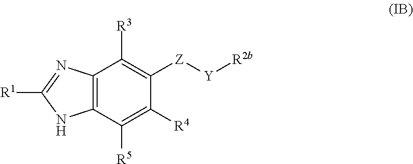

wherein R¹, R³, R⁴, R⁵, Y and Z are as defined for general formula (I) and:

R^{2b} is selected from:

i. a 3- to 10-membered carbocyclic ring system substituted with one or more substituents selected from fluoro; chloro; CN; nitro; OH; C₁₋₆ alkyl optionally substituted with one or more substituents selected from halo, OH and CN; O(C₁₋₆ alkyl) optionally substituted with one or more substituents selected from halo, OH and CN; and CH₂NH—C(O)O—C₁₋₆ alkyl optionally substituted with one or more substituents selected from halo and OH; or

Y and R^{2b} together form an unsubstituted C₃₋₈ alkyl group or a group CH₂—C(R^{17b})(R^{18b})—CH₂—N(R^{19b})R^{20b};
wherein each of R^{17b}, R^{18b} and R^{19b} is independently H or C₁₋₄ alkyl; and
R^{20b} is C₁₋₄ alkyl or C₁₋₄ haloalkyl;

provided that:
when R¹ is CH(R¹¹)(R¹²); where R¹² is phenyl and R¹¹ together with a substituent on R¹² and the atoms to which they are attached combine to form a 5- or 6-membered ring fused to the phenyl ring R¹², wherein the 5- or 6-membered ring is optionally substituted with C₁₋₃ alkyl:
   Y and R^{2b} do not combine to form C₃₋₈ alkyl.

In some compounds of general formula (IB), R^{2b} is a 3- to 10-membered carbocyclic ring system substituted as defined above.

In some compounds of this type, R^{2b} is a bridged carbocyclic ring system such as bicyclo[1.1.1]pentanyl, bicyclo[2.1.1]hexanyl, bicyclo-12.2.11heptanyl, bicyclo-12.2.2loctanyl or adamantyl, especially bicyclo-12.2.11heptanyl or adamantyl substituted as defined above. Compounds in which R² is substituted adamantyl are particularly suitable, especially adamantyl substituted with OH.

In other compounds of general formula (IB), R′ is a carbocyclic ring system, particularly a 5- to 8-membered carbocyclic ring system selected from cyclopentyl, cyclohexyl, cycloheptyl and cyclooctyl, any of which is substituted as defined above. More suitable substituents for such R² groups include OH, fluoro, C₁₋₆ alkyl, O(C₁₋₆ alkyl), and NH—C(O)O—C₁₋₆ alkyl, especially OH, C₁₋₄ alkyl, O(C₁₋₄ alkyl) and NH—C(O)O—C₁₋₄ alkyl, and still more suitably C₁₋₄ alkyl (especially methyl) fluoro and NH—C(O)O—C₁₋₄ alkyl.

In some compounds of general formula (IB), Y and R^{2b} together form an unsubstituted C₃₋₈ alkyl group, more suitably a C₅₋₈ alkyl group.

In some compounds of general formula (I), Y and R^{2b} together form a group CH₂—C(R^{17b})(R^{18b})—CH₂—N(R^{19b})R^{20b};
wherein each of R^{17b}, R^{18b}, R^{19b} and R^{20b} is as defined above.

More suitably, each of R^{17b}, R^{18b} and R^{19b} is independently H or methyl and R^{20b} is C₁₋₄ haloalkyl. Still more suitably, each of R^{17b} and R^{18b} is independently H or methyl, R^{19b} is H and R^{20b} is C₁₋₄ haloalkyl.

In the following discussion, references to a compound of general formula (I) includes compounds of general formulae (IA) and (IB).

Compounds of general formula (I) in which Z is —NH—C(O)— may be prepared by reacting a compound of general formula (II):

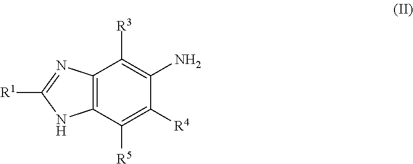

wherein R¹, R³, R⁴ and R⁵ are as defined for general formula (I);

with a compound of general formula (III):

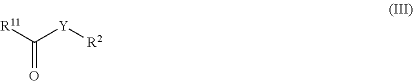

wherein Y and R² are as defined for general formula (I) and R¹¹ is OH or a halogen, particularly Cl.

When R¹¹ is a halogen, the reaction may be conducted in an organic solvent and in the presence of a base such as diisopropylethylamine.

When R¹¹ is OH, the reaction may be conducted in the presence of a coupling reagent and under basic conditions, for example in the presence of an amine such as diisopropylethylamine (DIPEA) or triethylamine (TEA) and in an organic solvent such as DMF.

Suitable coupling reagents include known peptide coupling agents such as 0-(Benzotriazol-1-yl)-N,N,N′,N′-tetramethyluronium hexafluorophosphate (HBTU), 0-(Benzotriazol-1-yl)- N,N,N′,N′- tetramethyluronium tetrafluoroborate (TBTU), O-(7-Azabenzotriazol -1-yl)-N,N,N ′,N′-tetramethyluronium hexafluorophosphate (HATU), O-(7-Azabenzotriazol-1-yl)- N,N,N′,N′-tetramethyluronium tetrafluoroborate (TATU), (Benzotriazol-1-yloxy)tris(dimethylamino)phosphonium hexafluorophosphate (BOP), (Benzotriazol-1-yloxy)tripyrrolidinophosphonium hexafluorophosphate (PyBOP) carbodiimides such as 1-ethyl-3-(3-dimethylaminopropyl)carbodiimide (EDCI) and triazoles such as 1-hydroxy-7-azabenzotriazole (HOAt) or hydroxybenzotriazole (HOBt). Suitably, the reaction is conducted under basic conditions, for example in the presence of an amine such as diisopropylethylamine (DIPEA) and in an organic solvent such as DMF

Compounds of general formula (III) are commercially available or may be prepared by known methods. This is also the case for some compounds of general formula (II). However, when compounds of general formula (II) are not commercially available, they may be prepared by reduction of a compound of general formula (IV):

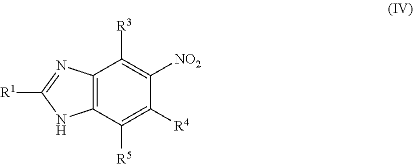

wherein R¹, R³, R⁴ and R⁵ are as defined for general formula (I).

The reduction may be carried out by hydrogenation over a palladium catalyst. Suitably, a hydrogenation reaction of this type will be conducted in an alcoholic solvent, for example ethanol.

Alternatively, the reduction may be carried out using a metal such as zinc and an acid such as acetic acid.

If R¹ of general formula (III) contains an OH group, it may be protected during the reaction, for example as a benzyloxy group. The protecting group may be removed during the reduction, particularly when hydrogenation is used.

A compound of general formula (IV) may be prepared by reacting a compound of general formula (V):

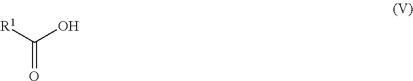

wherein R¹ is as defined for general formula (I);

with a compound of general formula (VI):

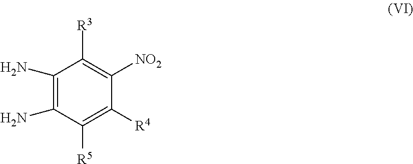

wherein R³, R⁴ and R⁵ are as defined for general formula (I).

The reaction has two steps. Suitably, the first step is carried out in the presence of a coupling reagent and under basic conditions, for example in the presence of an amine such as diisopropylethylamine (DIPEA) or triethylamine (TEA) and in an organic solvent such as DMF. Suitable coupling agents are as set out above for the reaction of the compounds of general formulae (II) and (III). The second step is a cyclisation step in which the product of the first step is heated in acidic conditions, suitably in acetic acid at temperatures of about 50 to 100° C.

Compounds of general formulae (V) and (VI) are commercially available or may be prepared using known methods.

An alternative method for the synthesis of a compound of general formula (I) in which Z is —NH—C(O)— is by reacting a compound of general formula (V) as defined above with a compound of general formula (VII)

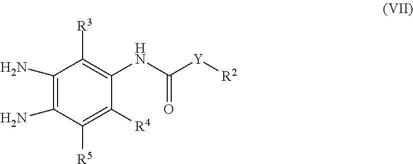

wherein R², R³, R⁴ and R⁵ are as defined above for general formula (I).

As with the reaction between compounds of general formulae (V) and (VI), the reaction is a two-step process. The first step is carried out in the presence of a coupling reagent and under basic conditions, for example in the presence of an amine such as diisopropylethylamine (DIPEA) or triethylamine (TEA) and in an organic solvent such as DMF. Suitable coupling agents are as set out above for the reaction of the compounds of general formulae (II) and (III). The second step is a cyclisation step and is carried out by heating the product of the first step with an acid such as acetic acid at a temperature of about 50 to 100° C.

A compound of general formula (VII) may be prepared by reduction of a compound of general formula (VIII):

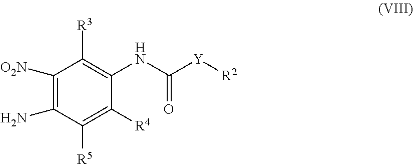

wherein R², R³, R⁴ and R⁵ are as defined above for general formula (I).

The reduction may be a catalytic hydrogenation, for example using a palladium catalyst in an alcoholic solvent such as ethanol.

A compound of general formula (VIII) may be prepared by reaction of a compound of general formula (III) as defined above with a compound of general formula (IX):

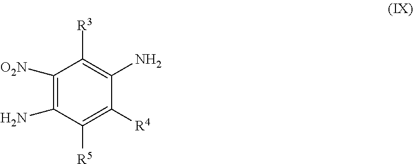

wherein R², R³, R⁴ and R⁵ are as defined above for general formula (I).

Suitably, the reaction is carried out in the presence of a coupling reagent and under basic conditions, for example in the presence of an amine such as diisopropylethylamine (DIPEA) or triethylamine (TEA) and in an organic solvent such as DMF. Suitable coupling agents are as set out above for the reaction of the compounds of general formulae (II) and (III).

Compounds of general formula (IX) are commercially available or may be prepared by known methods.

Compounds of general formula (I) in which Z is —C(O)—NH— may be prepared by reacting a compound of general formula (XII):

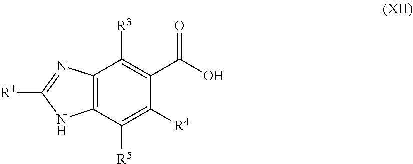

wherein R¹, R³, R⁴ and R⁵ are as defined for general formula (I);

with a compound of general formula (XIII):

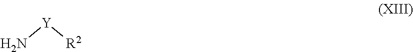

wherein R² and Y are as defined for general formula (I).

Suitably, the reaction is carried out in the presence of a coupling reagent and under basic conditions, for example in the presence of an amine such as 4-dimethylaminopyridine (DMAP) and in an organic solvent such as DMF. Suitable coupling agents are as set out above for the reaction of the compounds of general formulae (II) and (III), with 1-ethyl-3-(3-dimethylaminopropyl)carbodiimide (EDCI) being particularly suitable.

Compounds of general formula (XIII) are commercially available or may be prepared by known methods. Some compounds of general formula (XII) are also commercially available.

Compounds of general formula (XII) which are not commercially available may be prepared by hydrolysis of a compound of general formula (XIV):

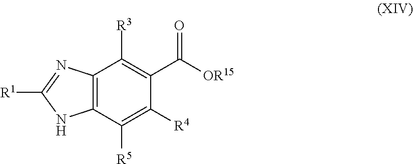

wherein RI, R³, R⁴ and R⁵ are as defined for general formula (I) and R¹⁵ is C₁₋₆ alkyl or benzyl.

The hydrolysis is suitably base hydrolysis for example using an alkali metal hydroxide, particularly lithium hydroxide, in aqueous solution.

Compounds of general formula (XIV) may be prepared by the reaction of a compound of general formula

(V) as defined above with a compound of general formula (XV):

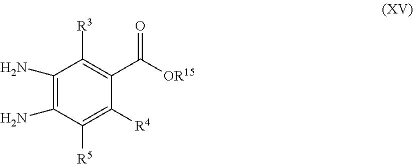

wherein R¹, R³, R⁴ and R⁵ are as defined for general formula (I) and R¹⁵ is as defined for general formula (XIV).

Suitably, the first step of the two step reaction is carried out in the presence of a coupling reagent and under basic conditions, for example in the presence of an amine such as diisopropylethylamine (DIPEA) or triethylamine (TEA) and in an organic solvent such as DMF. Suitable coupling agents are as set out above for the reaction of the compounds of general formulae (II) and (III). The product of the first step is then treated with an acid such as acetic acid.

Compounds of general formula (XV) are commercially available or may be prepared by known methods.

Compounds of general formula (I) in which Z is —C(O)—NH— may also be prepared by reacting compound of general formula (XIII) as defined above with a compound of general formula (XVI):

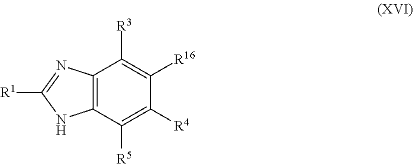

wherein R¹, R³, R⁴ and R⁵ are as defined for general formula (I) and R¹⁶ is a halogen, especially bromine; and carbon monoxide.

The carbon monoxide may be generated in situ as described in Example 3.3 below.

A compound of general formula (XVI) may be prepared by reaction of a compound of general formula (V) as defined above with a compound of general formula (XIX):

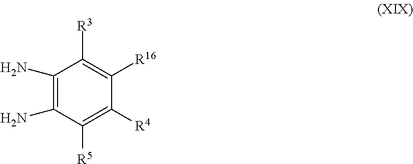

wherein R¹, R³, R⁴ and R⁵ are as defined for general formula (I) and R¹⁶ is as defined for general formula (XVI).

Suitably, the first step of the reaction is carried out in the presence of a coupling reagent and under basic conditions, for example in the presence of an amine such as diisopropylethylamine (DIPEA) or triethylamine (TEA) and in an organic solvent such as DMF. Suitable coupling agents are as set out above for the reaction of the compounds of general formulae (II) and (III). The cyclisation is achieved by treating the product of the first step with an acid such as acetic acid at a temperature of about 50 to 100° C.

Compounds of general formula (I) in which Z is —C(O)—NH— may also be prepared by reacting a compound of general formula (V) as defined above with a compound of general formula (XX)

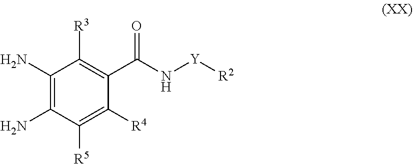

wherein R², R³, R⁴ and R⁵ are as defined above for general formula (I).

Suitably, the first step of the reaction is carried out in the presence of a coupling reagent and under basic conditions, for example in the presence of an amine such as diisopropylethylamine (DIPEA) or triethylamine (TEA) and in an organic solvent such as DMF. Suitable coupling agents are as set out above for the reaction of the compounds of general formulae (II) and (III). The cyclisation is achieved by treating the product of the first step with an acid such as acetic acid at a temperature of about 50 to 100° C.

A compound of general formula (XX) may be prepared by reacting a compound general formula (XIII) as defined above with a compound of general formula (XVIII):

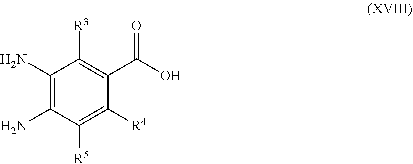

wherein R¹, R³, R⁴ and R⁵ are as defined for general formula (I) as defined above. Suitably, the reaction is carried out in the presence of a coupling reagent and under basic conditions, for example in the presence of an amine such as diisopropylethylamine (DIPEA) or triethylamine (TEA) and in an organic solvent such as DMF. Suitable coupling agents are as set out above for the reaction of the compounds of general formulae (II) and (III).

A compound of general formula (XVIII) may be prepared by hydrolysis of a compound of general formula (XV), in particular by base hydrolysis, for example using an alkali metal hydroxide such as lithium hydroxide in an alcoholic solvent such as methanol or a mixture of methanol, tetrahydrofuran and water.

In the synthesis of compounds of general formula (I), protecting groups may be used where necessary. Suitable protecting groups are well known (see Greene's Protective Groups in Organic Synthesis, Peter G. M. Wuts, Ed, John Wiley & Sons, Inc, 2014). For example, if the R¹ or R² group comprises an aromatic ring substituted with OH, protection may be required. For example, where the R¹ group is CH(R¹¹)(R¹²), in which R¹² is phenyl substituted with OH, the OH group may be protected as a lactone, which can be ring opened by treatment with a reducing agent, such as sodium or lithium borohydride, to give the required R¹² group. Alternatively, OH groups may be protected as O(C₁₋₆) alkyl, especially methoxy or as benzyloxy. When the protecting group is methoxy, deprotection may be is carried out by reaction with boron tribromide. Benzyloxy groups may be removed by catalytic hydrogenation as shown in Examples 1.4, 1.5 and 3.3. As a further alternative, OH groups may be protected by tri(C₁₋₆ alkyl) silyl groups, which may be removed in an aqueous workup.

The compounds of general formula (I) are positive modulators of TMEM16A and therefore, in a further aspect of the invention, there is provided a compound of general formula (I) as defined above for use in medicine, particularly in the treatment or prophylaxis of diseases and conditions affected by modulation of TMEM16A.

There is also provided the use of a compound of general formula (I) in the manufacture of a medicament for the treatment or prophylaxis of diseases and conditions affected by modulation of TMEM16A. There is also provided a method for the treatment or prophylaxis of diseases and conditions affected by modulation of TMEM16A, the method comprising administering to a patient in need of such treatment an effective amount of a compound of general formula (I).

The diseases and conditions affected by modulation of TMEM16A include respiratory diseases and conditions, dry mouth (xerostomia), intestinal hypermobility, cholestasis and ocular conditions.

There is also provided:
A compound of general formula (I) for use in the treatment or prophylaxis of respiratory diseases and conditions.
A compound of general formula (I) for use in the treatment or prophylaxis of dry mouth (xerostomia).
A compound of general formula (I) for use in the treatment or prophylaxis of intestinal hypermobility.
A compound of general formula (I) for use in the treatment or prophylaxis of cholestasis.
A compound of general formula (I) for use in the treatment or prophylaxis of ocular conditions.

The invention also provides:
The use of a compound of general formula (I) in the manufacture of a medicament for the treatment or prophylaxis of respiratory diseases and conditions.
The use of a compound of general formula (I) in the manufacture of a medicament for the treatment or prophylaxis of dry mouth (xerostomia).
The use of a compound of general formula (I) in the manufacture of a medicament for the treatment or prophylaxis of intestinal hypermobility.
The use of a compound of general formula (I) in the manufacture of a medicament for the treatment or prophylaxis of cholestasis.
The use of a compound of general formula (I) in the manufacture of a medicament for the treatment or prophylaxis of ocular conditions.

There is further provided:
A method for the treatment or prophylaxis of respiratory diseases and conditions, the method comprising administering to a patient in need of such treatment an effective amount of a compound of general formula (I).
A method for the treatment or prophylaxis of dry mouth (xerostomia), the method comprising administering to a patient in need of such treatment an effective amount of a compound of general formula (I).
A method for the treatment or prophylaxis of intestinal hypermobility, the method comprising administering to a patient in need of such treatment an effective amount of a compound of general formula (I).
A method for the treatment or prophylaxis of cholestasis, the method comprising administering to a patient in need of such treatment an effective amount of a compound of general formula (I).
A method for the treatment or prophylaxis of ocular conditions, the method comprising administering to a patient in need of such treatment an effective amount of a compound of general formula (I).

Respiratory diseases and conditions which may be treated or prevented by the compounds of general formula (I) include cystic fibrosis, chronic obstructive pulmonary disease (COPD), chronic bronchitis, emphysema, bronchiectasis, including non-cystic fibrosis bronchiectasis, asthma and primary ciliary dyskinesia.

Dry mouth (xerostomia) which may be treated or prevented by the compounds of general formula (I) may result from Sjorgens syndrome, radiotherapy treatment and xerogenic drugs.

Intestinal hypermobility which may be treated or prevented by the compounds of general formula (I) may be associated with gastric dyspepsia, gastroparesis, chronic constipation and irritable bowel syndrome.

Ocular conditions which may be treated or prevented by the compounds of by the compounds of general formula (I) include dry eye disease.

The compounds of the present invention will generally be administered as part of a pharmaceutical composition and therefore the invention further provides a pharmaceutical composition comprising a compound of general formula (I) together with a pharmaceutically acceptable excipient. The pharmaceutical composition may be formulated for oral, rectal, nasal, bronchial (inhaled), topical (including dermal, transdermal, eye drops, buccal and sublingual), vaginal or parenteral (including subcutaneous, intramuscular, intravenous and intradermal) administration and may be prepared by any methods well known in the art of pharmacy.

The composition may be prepared by bringing into association the above defined active agent with the excipient. In general, the formulations are prepared by uniformly and intimately bringing into association the active agent with liquid carriers or finely divided solid carriers or both, and then if necessary shaping the product. The invention extends to methods for preparing a pharmaceutical composition comprising bringing a compound of general formula (I) in conjunction or association with a pharmaceutically acceptable carrier or vehicle.

Formulations for oral administration in the present invention may be presented as: discrete units such as capsules, sachets or tablets each containing a predetermined amount of the active agent; as a powder or granules; as a solution or a suspension of the active agent in an aqueous liquid or a non-aqueous liquid; or as an oil-in-water liquid emulsion or a water in oil liquid emulsion; or as a bolus etc.

For compositions for oral administration (e.g. tablets and capsules), the term “acceptable carrier” includes vehicles such as common excipients e.g. binding agents, for example syrup, acacia, gelatin, sorbitol, tragacanth, polyvinylpyrrolidone (Povidone), methylcellulose, ethylcellulose, sodium carboxymethylcellulose, hydroxypropylmethylcellulose, sucrose and starch; fillers and carriers, for example con) starch, gelatin, lactose, sucrose, microcrystalline cellulose, kaolin, mannitol, dicalcium phosphate, sodium chloride and alginic acid; and lubricants such as magnesium stearate, sodium stearate and other metallic stearates, glycerol stearate, stearic acid, silicone fluid, talc waxes, oils and colloidal silica. Flavouring agents such as peppermint, oil of wintergreen, cherry flavouring and the like can also be used. It may be desirable to add a colouring agent to make the dosage form readily identifiable. Tablets may also be coated by methods well known in the art.

A tablet may be made by compression or moulding, optionally with one or more accessory ingredients. Compressed tablets may be prepared by compressing in a suitable machine the active agent in a free flowing form such as a powder or granules, optionally mixed with a binder, lubricant, inert diluent, preservative, surface-active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active agent.

Other formulations suitable for oral administration include lozenges comprising the active agent in a flavoured base, usually sucrose and acacia or tragacanth; pastilles comprising the active agent in an inert base such as gelatin and glycerin, or sucrose and acacia; and mouthwashes comprising the active agent in a suitable liquid carrier. For topical application to the skin, compounds of general formula (I) may be made up into a cream, ointment, jelly, solution or suspension etc. Cream or ointment formulations that may be used for the drug are conventional formulations well known in the art, for example, as described in standard text books of pharmaceutics such as the British Pharmacopoeia.

Topical administration to the lung may be achieved by use of an aerosol formulation. Aerosol formulations typically comprise the active ingredient suspended or dissolved in a suitable aerosol propellant, such as a chlorofluorocarbon (CFC) or a hydrofluorocarbon (HFC). Suitable CFC propellants include trichloromonofluoromethane (propellant 11), dichlorotetrafluoromethane (propellant 114), and dichlorodifluoromethane (propellant 12). Suitable HFC propellants include tetrafluoroethane (HFC-134a) and heptafluoropropane (HFC-227). The propellant typically comprises 40%-99.5% e.g. 40%-90% by weight of the total inhalation composition. The formulation may comprise excipients including co-solvents (e.g. ethanol) and surfactants (e.g. lecithin, sorbitan trioleate and the like). Other possible excipients include polyethylene glycol, polyvinylpyrrolidone, glycerine and the like. Aerosol formulations are packaged in canisters and a suitable dose is delivered by means of a metering valve (e.g. as supplied by Bespak, Valois or 3M or alternatively by Aptar, Coster or Vari).

Topical administration to the lung may also be achieved by use of a non-pressurised formulation such as an aqueous solution or suspension. These may be administered by means of a nebuliser e.g. one that can be hand-held and portable or for home or hospital use (ie non-portable). The formulation may comprise excipients such as water, buffers, tonicity adjusting agents, pH adjusting agents, surfactants and co-solvents. Suspension liquid and aerosol formulations (whether pressurised or unpressurised) will typically contain the compound of the invention in finely divided form, for example with a D₅₀ of 0.5-10 μm e.g. around 1-5 μm. Particle size distributions may be represented using D₁₀, D₅₀ and D₉₀ values. The D₅₀ median value of particle size distributions is defined as the particle size in microns that divides the distribution in half. The measurement derived from laser diffraction is more accurately described as a volume distribution, and consequently the D₅₀ value obtained using this procedure is more meaningfully referred to as a D_{V}_{₅₀} value (median for a volume distribution). As used herein D_{V} values refer to particle size distributions measured using laser diffraction. Similarly, D₁₀ and D₉₀ values, used in the context of laser diffraction, are taken to mean D_{V}_{₉₀} and D_{V}_{₉₀} values and refer to the particle size whereby 10% of the distribution lies below the D₁₀ value, and 90% of the distribution lies below the D₉₀ value, respectively.

Topical administration to the lung may also be achieved by use of a dry-powder formulation. A dry powder formulation will contain the compound of the disclosure in finely divided form, typically with a mass mean diameter (MMAD) of 1-10 μm or a D_{50 of} 0.5-10 μm e.g. around 1-5 μm. Powders of the compound of the invention in finely divided form may be prepared by a micronization process or similar size reduction process. Micronization may be performed using a jet mill such as those manufactured by Hosokawa Alpine.

The resultant particle size distribution may be measured using laser diffraction (e.g. with a Malvern Mastersizer 2000S instrument). The formulation will typically contain a topically acceptable diluent such as lactose, glucose or mannitol (preferably lactose), usually of comparatively large particle size e.g. a mass mean diameter (MMAD) of 50 am or more, e.g. 100 μm or more or a D₅₀ of 40-150 μm. As used herein, the term “lactose” refers to a lactose-containing component, including α-lactose monohydrate, β-lactose monohydrate, a-lactose anhydrous, β-lactose anhydrous and amorphous lactose. Lactose components may be processed by micronization, sieving, milling, compression, agglomeration or spray drying. Commercially available forms of lactose in various forms are also encompassed, for example Lactohale® (inhalation grade lactose; DFE Pharma), InhaLac 70 (sieved lactose for dry powder inhaler; Meggle), Pharmatose® (DFE Pharma) and Respitose® (sieved inhalation grade lactose; DFE Pharma) products. In one embodiment, the lactose component is selected from the group consisting of a-lactose monohydrate, α-lactose anhydrous and amorphous lactose. Preferably, the lactose is a-lactose monohydrate.

Dry powder formulations may also contain other excipients. Thus in one embodiment a dry powder formulation according the present disclosure comprises magnesium or calcium stearate. Such formulations may have superior chemical and/or physical stability especially when such formulations also contain lactose.

A dry powder formulation is typically delivered using a dry powder inhaler (DPI) device. Example dry powder delivery systems include SPINHALERO, DISKHALER®, TURBOHALER®, DISKUS®, SKYEHALER®, ACCUHALER® and CLICKHALER®. Further examples of dry powder delivery systems include ECLIPSE, NEXT, ROTAHALER, HANDIHALER, AEROLISER, CYCLOHALER, BREEZHALER/NEOHALER, MONODOSE, FLOWCAPS, TWINCAPS, X-CAPS, TURBOSPIN, ELPENHALER, MIATHALER, TWISTHALER, NOVOLIZER, PRESSAIR, ELLIPTA, ORIEL dry powder inhaler, MICRODOSE, PULVINAL, EASYHALER, ULTRAHALER, TAIFUN, PULMOJET, OMNIHALER, GYROHALER, TAPER, CONIX, XCELOVAIR and PROHALER.

In one embodiment a compound of general formula (I) is provided as a micronized dry powder formulation, for example comprising lactose of a suitable grade.

Thus, as an aspect of the invention there is provided a pharmaceutical composition comprising a compound of general formula (I) in particulate form in combination with particulate lactose, said composition optionally comprising magnesium stearate.

In one embodiment a compound of general formula (I) is provided as a micronized dry powder formulation, comprising lactose of a suitable grade and magnesium stearate, filled into a device such as DISKUS. Suitably, such a device is a multidose device, for example the formulation is filled into blisters for use in a multi-unit dose device such as DISKUS.

In another embodiment a compound of general formula (I) is provided as a micronized dry powder formulation, for example comprising lactose of a suitable grade, filled into hard shell capsules for use in a single dose device such as AEROLISER.

In another embodiment a compound of general formula (I) is provided as a micronized dry powder formulation, comprising lactose of a suitable grade and magnesium stearate, filled into hard shell capsules for use in a single dose device such as AEROLISER.

In another embodiment a compound of general formula (I) is provided as a fine powder for use in an inhalation dosage form wherein the powder is in fine particles with a D₅₀ of 0.5-10 μm e.g. around 1-5 μm, that have been produced by a size reduction process other than jet mill micronisation e.g. spray drying, spray freezing, microfluidisation, high pressure homogenisation, super critical fluid crystallisation, ultrasonic crystallisation or combinations of these methods thereof, or other suitable particle formation methods known in the art that are used to produce fine particles with an aerodynamic particle size of 0.5-10 μm. The resultant particle size distribution may be measured using laser diffraction (e.g. with a Malvern Mastersizer 2000S instrument). The particles may either comprise the compound alone or in combination with suitable other excipients that may aid the processing. The resultant fine particles may form the final formulation for delivery to humans or may optionally be further formulated with other suitable excipients to facilitate delivery in an acceptable dosage form.

The compound of the invention may also be administered rectally, for example in the form of suppositories or enemas, which include aqueous or oily solutions as well as suspensions and emulsions and foams. Such compositions are prepared following standard procedures, well known by those skilled in the art. For example, suppositories can be prepared by mixing the active ingredient with a conventional suppository base such as cocoa butter or other glycerides. In this case, the drug is mixed with a suitable non-irritating excipient which is solid at ordinary temperatures but liquid at the rectal temperature and will therefore melt in the rectum to release the drug. Such materials are cocoa butter and polyethylene glycols.

Generally, for compositions intended to be administered topically to the eye in the form of eye drops or eye ointments, the total amount of the compound of general formula (I) will be about 0.0001 to less than 4.0% (w/w).

Preferably, for topical ocular administration, the compositions administered according to general formula (I) will be formulated as solutions, suspensions, emulsions and other dosage forms. Aqueous solutions are generally preferred, based on ease of formulation, as well as a patient's ability to administer such compositions easily by means of instilling one to two drops of the solutions in the affected eyes. However, the compositions may also be suspensions, viscous or semi-viscous gels, or other types of solid or semi-solid compositions. Suspensions may be preferred for compounds that are sparingly soluble in water.

An alternative for administration to the eye is intravitreal injection of a solution or suspension of the compound of general formula (I). In addition, the compound of general formula (I) may also be introduced by means of ocular implants or inserts.

The compositions administered according to general formula (I) may also include various other ingredients, including, but not limited to, tonicity agents, buffers, surfactants, stabilizing polymer, preservatives, co-solvents and viscosity building agents. Suitable pharmaceutical compositions of general formula (I) include a compound of the invention formulated with a tonicity agent and a buffer. The pharmaceutical compositions of general formula (I) may further optionally include a surfactant and/or a palliative agent and/or a stabilizing polymer.

Various tonicity agents may be employed to adjust the tonicity of the composition, preferably to that of natural tears for ophthalmic compositions. For example, sodium chloride, potassium chloride, magnesium chloride, calcium chloride, simple sugars such as dextrose, fructose, galactose, and/or simply polyols such as the sugar alcohols mannitol, sorbitol, xylitol, lactitol, isomaltitol, maltitol, and hydrogenated starch hydrolysates may be added to the composition to approximate physiological tonicity. Such an amount of tonicity agent will vary, depending on the particular agent to be added. In general, however, the compositions will have a tonicity agent in an amount sufficient to cause the final composition to have an ophthalmically acceptable osmolality (generally about 150-450 mOsm, preferably 250-350 mOsm and most preferably at approximately 290 mOsm). In general, the tonicity agents of the invention will be present in the range of 2 to 4% w/w. Preferred tonicity agents of the invention include the simple sugars or the sugar alcohols, such as D-mannitol.

An appropriate buffer system (e.g. sodium phosphate, sodium acetate, sodium citrate, sodium borate or boric acid) may be added to the compositions to prevent pH drift under storage conditions. The particular concentration will vary, depending on the agent employed. Preferably however, the buffer will be chosen to maintain a target pH within the range of pH 5 to 8, and more preferably to a target pH of pH 5 to 7.

Surfactants may optionally be employed to deliver higher concentrations of compound of general formula (I). The surfactants function to solubilise the compound and stabilise colloid dispersion, such as micellar solution, microemulsion, emulsion and suspension. Examples of surfactants which may optionally be used include polysorbate, poloxamer, polyosyl 40 stearate, polyoxyl castor oil, tyloxapol, Triton, and sorbitan monolaurate. Preferred surfactants to be employed in the invention have a hydrophile/lipophile/balance “HLB” in the range of 12.4 to 13.2 and are acceptable for ophthalmic use, such as TritonX114 and tyloxapol.

Additional agents that may be added to the ophthalmic compositions of compounds of general formula (I) are demulcents which function as a stabilising polymer. The stabilizing polymer should be an ionic/charged example with precedence for topical ocular use, more specifically, a polymer that carries negative charge on its surface that can exhibit a zeta-potential of (−)10-50 mV for physical stability and capable of making a dispersion in water (i.e. water soluble). A preferred stabilising polymer of the invention would be polyelectrolyte, or polyelectrolytes if more than one, from the family of cross-linked polyacrylates, such as carbomers and Pemulen(R), specifically Carbomer 9′74p (polyacrylic acid), at 0.1-0.5% w/w.

Other compounds may also be added to the ophthalmic compositions of the compound of general formula (I) to increase the viscosity of the carrier. Examples of viscosity enhancing agents include, but are not limited to: polysaccharides, such as hyaluronic acid and its salts, chondroitin sulfate and its salts, dextrans, various polymers of the cellulose family; vinyl polymers; and acrylic acid polymers.

Topical ophthalmic products are typically packaged in multidose form. Preservatives are thus required to prevent microbial contamination during use. Suitable preservatives include: benzalkonium chloride, chlorobutanol, benzododecinium bromide, methyl paraben, propyl paraben, phenylethyl alcohol, edentate disodium, sorbic acid, polyquaternium-1, or other agents known to those skilled in the art. Such preservatives are typically employed at a level of from 0.001 to 1.0% w/v. Unit dose compositions of general formula (I) will be sterile, but typically unpreserved. Such compositions, therefore, generally will not contain preservatives.

Parenteral formulations will generally be sterile.

The medical practitioner, or other skilled person, will be able to determine a suitable dosage for the compound of general formula (I) and hence the amount of the compound of the invention that should be included in any particular pharmaceutical formulation (whether in unit dosage form or otherwise).

Compounds of general formula (I) may be used in combination with one or more other active agents which are useful in the treatment or prophylaxis of respiratory diseases and conditions.

An additional active agent of this type may be included in the pharmaceutical composition described above but alternatively it may be administered separately, either at the same time as the compound of general formula (I) or at an earlier or later time.

Therefore, in a further aspect of the present invention there is provided a product comprising a compound of general formula (I) and an additional agent useful in the treatment or prevention of respiratory conditions as a combined preparation for simultaneous, sequential or separate use in the treatment of a disease or condition affected by modulation of TMEM16A and especially a respiratory disease or condition, for example one of the diseases and conditions mentioned above.

There is also provided a compound of general formula (I) in combination with an additional agent useful in the treatment or prevention of respiratory conditions as a combined preparation for simultaneous, sequential or separate use in the treatment of a disease or condition affected by modulation of TMEM16A and especially a respiratory disease or condition, for example one of the diseases and conditions mentioned above.

Suitable additional active agents which may be included in a pharmaceutical composition or a combined preparation with the compounds of general formula (I) include:
β2 adrenoreceptor agonists such as metaproterenol, isoproterenol, isoprenaline, albuterol, salbutamol, formoterol, salmeterol, indacaterol, terbutaline, orciprenaline, bitolterol mesylate, pirbuterol, olodaterol, vilanterol and abediterol;
antihistamines, for example histamine H1 receptor antagonists such as loratadine, cetirizine, desloratadine, levocetirizine, fexofenadine, astemizole, azelastine and chlorpheniramine or H4 receptor antagonists; dornase alpha;
corticosteroids such as prednisone, prednisolone, flunisolide, triamcinolone acetonide, beclomethasone dipropionate, budesonide, fluticasone propionate mometasone furoate and fluticasone furoate; Leukotriene antagonists such as montelukast and zafirlukast;
anticholinergic compounds, particularly muscarinic antagonists such as ipratropium, tiotropium, glycopyrrolate, aclidinium and umeclidinium;
CFTR repair therapies (e.g. CFTR potentiators, correctors or amplifiers) such as Ivacaftor, QBW251, Bamacaftor (VX659), Elexacaftor (VX445), VX561/CPT-656, VX152, Olacaftor (VX440), GLP2737, GLP2222, GLP2451, PTI438, PTI801, PTI808, FDL-169 and FDL-176 and CFTR correctors such as Lumacaftor and Tezacaftor or combinations thereof (for example a combination of Ivacaftor, Tezacaftor and Elexacaftor);
ENaC modulators, particularly ENaC inhibitors;
Antibiotics;
Antivirals such as ribavirin and neuraminidase inhibitors such as zanamivir;
Antifungals such as PUR1900;
Airway hydrating agents (osmoloytes) such as hypertonic saline and mannitol (Bronchitol®); and Mucolytic agents such as. N-acetyl cysteine.

When the additional active agent is an ENaC modulator, it may be an ENaC inhibitor such as amiloride, VX-371, AZD_{5634,} QBW276, SPX-101, BI443651, BI1265162 and ETD_{001.} Other suitable ENaC blockers are disclosed in WO 2017/221008, WO 2018/096325, WO 2019/077340 and WO 2019/220147 and any of the example compounds of those applications may be used in combination with the compounds of general formula (I). Particularly suitable compounds for use in combination with the compounds of general formula (I) include compounds having a cation selected from:
2-[({3-amino-5H-pyrrolo [2,3-b]pyrazin-2-yl}formamido) ethyl]-6-(4-{bis[(2S,3R,4R,5R)-2,3,4,5 ,6-pentahydroxyhexyl]amino}piperidine-1-carbonyl)-1,3-diethyl-1H-1, 3-benzodiazol-3-ium;
2-[({3-amino-5H-pyrrolo [2,3-b]pyrazin-2-yl}formamido) methyl]-6-{[2-(4-{bis[(2S,3R,4R,5R)-2,3,4,5,6-pentahydroxyhexyl] amino Ipiperidin-1-ypethyl}carbamoyl }-1,3-diethyl-1H-1,3-benzodiazol-3-ium;
2-[({3-amino-5H-pyrrolo [2,3-b]pyrazin-2-yl}formamido)methyl]-5[4-( {bis[(2S,3R,4R,5R)-2,3,4,5,6-pentahydroxyhexyl]amino}methyppiperidine -1-carbonyl]-1,3-diethyl -1H-1,3-benzodi azol-3-ium;
2-[({3-amino-5H-pyrrolo [2,3-b]pyrazin-2-yl}formamido)methyl]-6-[(3R)-3-{bis[(2S,3R,4R,5R)-2,3,4,5,6-pentahydroxyhexyl]amino}pyrrolidine -1-carbonyl]-1,3-diethyl-1H-1,3-benzodi azol-3-ium;
2-[({3-amino-5H-pyrrolo [2,3-b] pyrazin-2-yl }formamido)methyl]-6-[(3S)-3-{bis[(2S,3R,4R,5R)-2,3,4,5,6-pentahydroxyhexyl]amino}pyrrolidine -1-carbonyl]-1,3-diethyl-1H-1,3-benzodi azol-3-ium;
2-[({3-amino-5H-pyrrolo [2,3-b]pyrazin-2-yl}formamido)methyl]-1,3-diethyl-6-{[(1r,4r)-4-{bis [(2S,3R,4R,5R)-2,3,4,5,6-pentahydroxyhexyl]amino}cyclohexyl]carbamoyl }-1H-1,3-benzodiazol-3-ium;
2-[({3-amino-5H-pyrrolo [2,3-b]pyrazin-2-yl}formamido)methyl]-1,3-diethyl-6-{[(1s,4s)-4-{bis[(2S,3R,4R,5R)-2,3,4,5,6-pentahydroxyhexyl]amino}cyclohexyl]carbamoyl}-1H-1,3-benzodiazol-3-ium;
and a suitable anion, for example halide, sulfate, nitrate, phosphate, formate, acetate, trifluoroacetate, fumarate, citrate, tartrate, oxalate, succinate, mandelate, methane sulfonate or p-toluene sulfonate.

The invention is illustrated by the following Examples.

### EXAMPLES

The invention is illustrated by the following non-limiting Examples.

### Examples

### General Conditions

Mass spectra were run on LC-MS systems using electrospray ionization. These were run using either a Waters Acquity uPLC system with Waters PDA and ELS detectors or Shimadzu LCMS-2010EV systems. [M+H]+ refers to mono-isotopic molecular weights.

NMR spectra were recorded on a Bruker Avance III HD 500 MHz with a 5 mm Broad Band Inverse probe, a Bruker Avance III HD 250 MHz or a 400 MHz Avance III HD Nanobay fitted with a 5 mm Broad Band Observed SmartProbe using the solvent as internal deuterium lock. Spectra were recorded at room temperature unless otherwise stated and were referenced using the solvent peak.

Referring to the examples that follow, compounds of the preferred embodiments were synthesized using the methods described herein, or other methods, which are known in the art.

The various starting materials, intermediates, and compounds of the preferred embodiments may be isolated and purified, where appropriate, using conventional techniques such as precipitation, filtration, crystallization, evaporation, distillation, and chromatography. Unless otherwise stated, all starting materials are obtained from commercial suppliers and used without further purification. Salts may be prepared from compounds by known salt-forming procedures.

Compounds were purified by flash column chromatography on normal phase silica on Biotage® Isolera systems using the appropriate SNAP cartridge or Sfar cartridge and gradient. Alternatively, compounds were purified on reverse phase silica using either Biotage® Isolera or Biotage® Selekt systems with the appropriate SNAP C18 or Sfax C18 cartridges and reverse phase eluent or by preparative HPLC (if stated otherwise).

### Preparative HPLC Using Acidic pH, Early Elution Method

Purifications by were performed on a Gilson LC system using Waters Sunfire C18 columns (30 mm×100 mm, 10 μM; temperature: RT) and a gradient of 10-95% B (A=0.1% formic acid in water; B=0.1% formic acid in acetonitrile) over 14.44 min then 95% B for 2.11 min, with an injection volume of 1500 uL and a flow rate of 40 mL/min. UV spectra were recorded at 215 nm using a Gilson detector.

### Preparative HPLC Using Acidic pH, Standard Elution Method

Purifications by preparative HPLC (acidic pH, standard elution method) were performed on a Gilson LC system using Waters Sunfire C18 columns (30 mm×100 mm, 10 μM; temperature: RT) and a gradient of 30-95% B (A=0.1% formic acid in water; B=0.1% formic acid in acetonitrile) over 11 min then 95% B for 2.11 min, with an injection volume of 1500 μL and a flow rate of 40 mL/min. UV spectra were recorded at 215 nm using a Gilson detector.

### Preparative HPLC Using Basic pH, Early Elution Method

Purifications by preparative HPLC (basic pH, early elution method) were performed on a Gilson LC system using Waters Xbridge C18 columns (30 mm×100 mm, 10 μM; temperature: RT) and a gradient of 10-95% (A=0.2% ammonium hydroxide in water; B=0.2% ammonium hydroxide in acetonitrile) over 14.44 min then 95% B for 2.11 min, with an injection volume of 1500 μL and a flow rate of 40 mL/min. UV spectra were recorded at 215 nm using a Gilson detector.

### Preparative HPLC Using Basic pH, Standard Elution Method

Purifications by preparative HPLC (basic pH, standard elution method) were performed on a Gilson LC system using Waters Xbridge C18 columns (30 mm×100 mm, 10 μM; temperature: RT) and a gradient of 30-95% (A=0.2% ammonium hydroxide in water; B=0.2% ammonium hydroxide in acetonitrile) over 11 min then 95% B for 2.11 min, with an injection volume of 1500 μL and a flow rate of 40 mL/min. UV spectra were recorded at 215 nm using a Gilson detector.

If not indicated otherwise, the analytical HPLC conditions are as follows:

### Method A

Column: Phenomenex Kinetix-XB C18 2.1×100 mm, 1.7 μm
Column Temp: 40° C.
Eluents: A: H₂O+0.1% formic acid, B: acetonitrile+0.1% formic acid
Flow Rate: 0.6 mL/min
Gradient: 0-5.3 min 5-100% B, 5.3-5.8 min 100% B, 5.8-5.82 min 100-5% B, 5.82-7.00 min 5% B

### Method B

Column: Waters UPLC® CSH™ C18 2.1×100 mm, 1.7 μm
Column Temp: 40° C.
Eluents: A: 2 mM amm.bicarbonate, buffered to pH10, B: acetonitrile
Flow Rate: 0.6 mL/min
Gradient: 0-5.3 min 5-100% B, 5.3-5.8 min 100% B, 5.8-5.82 min 100-5% B, 5.82-7.00 min 5% B

### Method C

Column: Waters UPLC® BEH TM C18 2.1×100 mm, 1.7 μm
Column Temp: 40° C.
Eluents: A: 2 mM ammonium bicarbonate, buffered to pH10, B: acetonitrile
Flow Rate: 0.6 mL/min
Gradient: 0-5.3 min 5-100% B, 5.3-5.8 min 100% B, 5.8-5.82 min 100-5% B, 5.82-7.00 min 5% B

### Method E

Column: Kinetex Core-Shell C18 2.1×50 mm, 5μm
Column Temp: 40° C.
Eluents: A: H₂O+0.1% formic acid, B: acetonitrile+0.1% formic acid
Flow Rate: 1.2 mL/min
Gradient: 0-1.20 min 5-100% B, 1.20-1.30 min 100% B, 1.30-1.31 min 100-5% B, 1.31-1.7 min 5% B

### Method F

Column: Phenomenex Gemini-NX C18 2×50 mm, 3 μm
Column Temp: 40° C.
Eluents: A: 2 mM ammonium bicarbonate, buffered to pH10, B: acetonitrile
Flow Rate: 1 mL/min
Gradient: 0-1.80 min 1-100% B, 1.80-2.10 min 100% B, 2.10-2.30 min 100-1% B, 2.30-3.50 min 1% B

The following examples are intended to illustrate the invention and are not to be construed as being limitations thereon. Temperatures are given in degrees centigrade. If not mentioned otherwise, all evaporations are performed in vacuo, preferably between about 15 mm Hg and 100 mm Hg (=20-133 mbar). The structure of final products, intermediates and starting materials is confirmed by standard analytical methods, e.g., microanalysis and spectroscopic characteristics, e.g., MS, IR, and NMR. Abbreviations used are those conventional in the art. If not defined, the terms have their generally accepted meanings.

### Abbreviation

AcOH acetic acid
aq. aqueous
br broad
d doublet
dd doublet of doublets
DCE dichloroethane
DCM dichloromethane
DIPEA diisopropylethylamine
DMAP 4-dimethylaminopyridine
DMF N,N-dimethylformamide
EDCI 1-ethyl-3-(3-dimethylaminop ropyl)carbodiimide
EtOAc ethyl acetate
EtOH ethanol
HOAt 1-hydroxy-7-azabenzotriazole
HATU 2-(7-aza-1H-benzotriazole-1-yl)-1,1,3,3-tetramethyluronium hexafluorophosphate
HPLC high pressure liquid chromatography
IPA isopropyl alcohol
MeCN acetonitrile
MeOH MeOH
MS mass spectrometry
m multiplet
min minute(s)
mL milliliter(s)
m/z mass to charge ratio
NMR nuclear magnetic resonance
q quartet
Rt retention time
s singlet
t triplet
N,N,N′,N′-tetramethyl-O-(benzotriazol-1-yl)uronium tetrafluoroborate
TEA triethylamine
THF tetrahydrofuran

### Preparation of Examples

### Example 1.1

### N-(2-Benzyl-1H-benzimidazol-5-yl)-2-cyclohexyl-acetamide

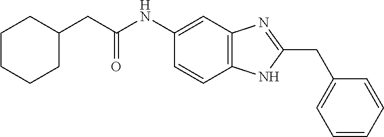 [SMILES: O=C(CC1CCCCC1)Nc1ccc2[nH]c(Cc3ccccc3)nc2c1]

To a cooled (0° C.) solution of 2-benzyl-1H-benzimidazol-5-amine (Intermediate A) (75 mg, 0.34 mmol) and DIPEA (70 μL, 0.40 mmol) in DCM (2 mL) was added 2-cyclohexylacetyl chloride (57 μL, 0.37 mmol). The solution was warmed to room temperature and stirred for 1 h. The resulting mixture was diluted with DCM (5 mL) and washed with water (5 mL), brine (5 mL) dried over Na₂SO₄ and concentrated in vacuo. The crude residue was dissolved in MeOH (2 mL) and 7M NH 3 in MeOH (0.5 mL) and the mixture was allowed to stand for 5 mins. The methanolic solution was purified by preparative HPLC (basic pH, early elution method) to afford the title compound as off-white powder.

LC-MS (Method A): Rt 2.10 min; MS m/z 348.2=[M+H]+

1H NMR (500 MHz, DMSO-d6) δ12.16-12.08 (m, 1H), 9.80-9.68 (m, 1H), 7.97-7.83 (m, 1H), 7.42-7.09 (m, 7H), 4.13 (s, 2H), 2.17 (d, J=7.0 Hz, 2H), 1.82-1.74 (m, 1H), 1.72-1.59 (m, 5H), 1.27-1.11 (m, 3H), 1.02-0.93 (m, 2H).

### Example 1.2

### 2-Benzyl-N-(cyclohexylmethyl)-1H-benzimidazole-5-carboxamide

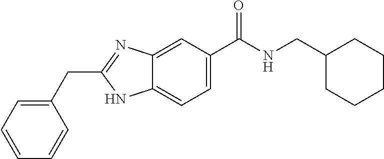 [SMILES: O=C(NCC1CCCCC1)c1ccc2[nH]c(Cc3ccccc3)nc2c1]

To a solution of commercially available 2-benzyl-1H-benzimidazole-5-carboxylic acid (75 mg, 0.30 mmol) in DMF (2 mL) was added EDCI (63 mg, 0.33 mmol), DMAP (40 mg, 0.33 mmol) and HOAt (45 mg, 0.33 mmol). After stirring at room temperature for 5 mins, cyclohexylmethanamine (67 mg, 0.59 mmol) was added and stirring was continued under an inert atmosphere for 16 h. The resulting mixture was diluted with EtOAc (20 mL) and washed with water (2×10 mL), brine (2×10 mL) and concentrated in vacuo. The crude material was purified by preparative HPLC (acidic pH, standard elution method) to afford the title compound as a colourless solid.

LC-MS (Method A): Rt 2.21 min; MS m/z 348.2=[M+H]+

1H NMR (500 MHz,Methanol-d4) δ 8.43 (t, J=5.7 Hz, 1H), 8.01 (s, 1H), 7.70 (dd, J=8.5, 1.6 Hz, 1H), 7.54 (d, J=8.4 Hz, 1H), 7.35-7.30 (m, 4H), 7.28-7.23 (m, 1H), 4.26 (s, 2H), 3.26-3.21 (m, 2H), 1.85 -1.73 (m, 4H), 1.71-1.62 (m, 2H), 1.34-1.17 (m, 3H), 1.07-0.97 (m, 2H).

### Example 1.2.1

### N-(1-Adam antylmethyl)-2-benzyl-1H-benzimidazole-5-carboxamide

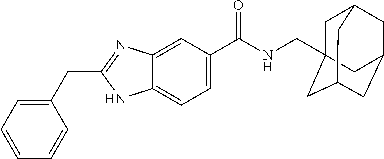 [SMILES: O=C(NCC12CC3CC(CC(C3)C1)C2)c1ccc2[nH]c(Cc3ccccc3)nc2c1]

The title compound was prepared from 2-benzyl-1H-benzimidazole-5-carboxylic acid and 1-adamantylmethanamine analogously to Example 1.2.

LC-MS (Method A): Rt 2.67 min; MS m/z 400.3=[M+H]+

1H NMR (500 MHz, DMSO-d6) δ 12.47 (br. s, 1H), 8.21-8.15 (m, 1H), 8.02 (br. s, 1H), 7.67 (d, J=8.4 Hz, 1H), 7.56-7.39 (m, 1H), 7.35-7.29 (m, 4H), 7.26-7.20 (m, 1H), 4.20 (s, 2H), 3.00 (d, J=6.3 Hz, 2H), 1.96-1.89 (m, 3H), 1.69-1.56 (m, 6H), 1.54-1.46 (m, 6H).

### Example 1.2.2

### 2-Benzyl-N- I(1-methylcyclopentyl)methylj -1H-benzimidazole-5-carboxamide

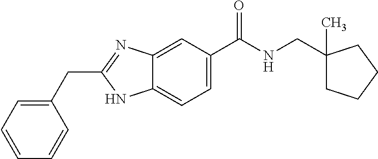 [SMILES: CC1(CNC(=O)c2ccc3[nH]c(Cc4ccccc4)nc3c2)CCCC1]

The title compound was prepared from 2-benzyl-1H-benzimidazole-5-carboxylic acid and (1-methylcyclopentyl)methanamine analogously to Example 1.2.

LC-MS (Method A): Rt 2.22 min; MS m/z 348.2=[M+H]+

1H NMR (500 MHz, DMSO-d6) δ 12.48 (br s, 1H), 8.26 (t, J=6.0 Hz, 1H), 8.19-7.80 (m, 1H), 7.67 (d, J=8.1 Hz, 1H), 7.61-7.39 (m, 1H), 7.38-7.29 (m, 4H), 7.28-7.19 (m, 1H), 4.20 (s, 2H), 3.22 (d, J=6.3 Hz, 2H), 1.69-1.51 (m, 6H), 1.32-1.17 (m, 2H), 0.98 (s, 3H).

### Example 1.2.3

### 2-Benzyl-N-[(1R)-1-cyclohexylethyl]-1H-benzimidazole-5-carboxamide

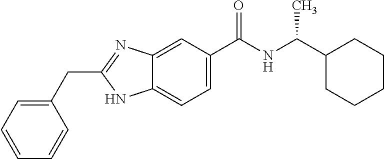 [SMILES: C[C@@H](NC(=O)c1ccc2[nH]c(Cc3ccccc3)nc2c1)C1CCCCC1]

The title compound was prepared from 2-benzyl-1H-benzimidazole-5-carboxylic acid and (1R)-1-cyclohexylethanamine analogously to Example 1.2.

LC-MS (Method A): Rt 2.38 min; MS m/z 362.3=[M+H]+

1H NMR (500 MHz, DMSO-d6) δ 12.45 (br s, 1H), 8.15-7.83 (m, 2H), 7.66 (d, J=8.5 Hz, 1H), 7.47 (br s, 1H), 7.36-7.28 (m, 4H), 7.27-7.17 (m, 1H), 4.19 (s, 2H), 3.89-3.79 (m, 1H), 1.78-1.66 (m, 4H), 1.63-1.57 (m, 1H), 1.46-1.38 (m, 1H), 1.24-1.07 (m, 6H), 1.00-0.89 (m, 2H).

### Example 1.3

### N-(Cycloheptylmethyl)-2-(1,1-dimethylpropyl)-3H-benzimidazole-5-carboxamide

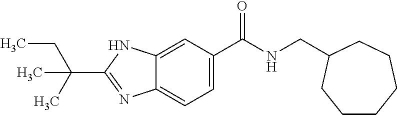 [SMILES: CCC(C)(C)c1nc2ccc(C(=O)NCC3CCCCCC3)cc2[nH]1]

### Step 1: 3,4-Diamino-N-(cycloheptylmethyl)benzamide

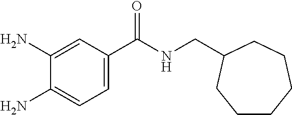 [SMILES: Nc1ccc(C(=O)NCC2CCCCCC2)cc1N]

To a solution of cycloheptylmethanamine (2.27 mL, 15.77 mmol) and 3,4-diaminobenzoic acid (2.0 g, 13.14 mmol) in THF (50 mL) and DMF (20 mL) was added TBTU (5.06 g, 15.77 mmol) and TEA (5.5 mL, 39.43 mmol). After stirring at room temperature for 19 h, the mixture was concentrated in vacuo. The crude material was dissolved in EtOAc (50 mL) and washed with water (2×25 mL). The aqueous portions were back-extracted with EtOAc (3×50 mL) and the combined organic extracts were washed with brine (2×25 mL), dried over Na₂SO₄ and concentrated in vacuo to afford a dark brown syrup. Purification of the crude product by C18 reverse phase chromatography eluting with 10-100% MeCN in water afforded the title compound as a pale brown solid.

LC-MS (Method E): Rt 0.96 min; MS m/z 262.1=[M+H]+

1H NMR (250 MHz, Chloroform-d) δ 7.22 (d, J=1.9 Hz, 1H), 7.08 (dd, J=8.0, 2.0 Hz, 1H), 6.67 (d, J=8.0 Hz, 1H), 6.13-5.92 (m, 1H), 3.81-2.90 (m, 6H), 1.85-1.33 (m, 11H), 1.32-1.14 (m, 2H).

Step 2: N-(Cycloheptylmethyl)-2-(1,1-dimethylpropyl)-3H-benzimidazole -5-carb oxamide A mixture of 2,2-dimethylbutanoic acid (34 μL, 0.28 mmol), HATU (105 mg, 0.28 mmol) and TEA (80 μL, 0.46 mmol) in DMF (1.9 mL) was stirred at room temperature for 1 h and then treated with 3,4-diamino-N-(cycloheptylmethyl)benzamide (step 1) (60 mg, 0.23 mmol) in DMF (1 mL). After stirring at room temperature for 24 h, the mixture was diluted with EtOAc (10 mL) and washed with saturated aqueous sodium hydrogen carbonate (2×10 mL). The organic portion was dried over Na₂SO₄ and concentrated in vacuo and the crude material was purified by chromatography on silica eluting with 0-100% EtOAc in heptanes. The resulting residue was dissolved in acetic acid (1.9 mL) and stirred at 60° C. for 3 h. The mixture was diluted with EtOAc (20 ml) and washed with saturated aqueous sodium hydrogen carbonate (3×20 mL). The organic portion was dried over Na₂SO₄ and concentrated in vacuo. Purification of the residue by preparative HPLC (low pH, early elution method) afforded the title compound as a colourless solid.

LC-MS (Method A): Rt 2.22 min; MS m/z 342=[M+H]+

1H NMR (500 MHz, DMSO-d6) δ 12.27-12.17 (m, 1H), 8.41-8.25 (m, 1H), 8.11-7.86 (m, 1H), 7.70-7.58 (m, 1H), 7.58-7.39 (m, 1H), 3.11 (t, J=6.3 Hz, 2H), 1.79-1.69 (m, 5H), 1.67-1.59 (m, 2H), 1.58-1.44 (m, 4H), 1.43-1.33 (m, 8H), 1.22-1.13 (m, 2H), 0.70 (t, J=7.4 Hz, 3H).

### Example 1.3.1

### N-(Cycloheptylmethyl)-2-[(1-hydroxycyclohexyl)methyl]-1H-benzimidazole-5-carboxamide

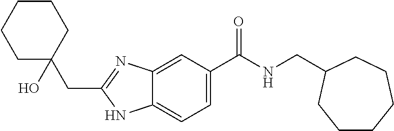 [SMILES: O=C(NCC1CCCCCC1)c1ccc2[nH]c(CC3(O)CCCCC3)nc2c1]

The title compound was prepared from 3,4-diamino-N-(cycloheptylmethyl)benzamide (Example 1.3 step 1) and 2-(1-hydroxycyclohexyl)acetic acid analogously to Example 1.3 step 2.

LC-MS (Method A): Rt 2.19 min; MS m/z 384.4=[M+H]+

1H NMR (500 MHz, DMSO-d6) δ 12.21 (br. s, 1H), 8.35 (t, J=5.6 Hz, 1H), 8.02 (s, 1H), 7.65 (d, J=8.3 Hz, 1H), 7.50 (d, J=7.7 Hz, 1H), 4.67 (br. s, 1H), 3.10 (t, J=6.3 Hz, 2H), 2.92 (s, 2H), 1.80-1.70 (m, 3H), 1.68-1.34 (m, 17H), 1.23-1.12 (m, 3H).

### Example 1.3.2

### N-(Cycloheptylmethyl)-2-(2-hydroxy-1-phenyl-ethyl)-1H-benzimidazole-5-carboxamide

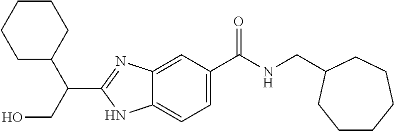 [SMILES: O=C(NCC1CCCCCC1)c1ccc2[nH]c(C(CO)C3CCCCC3)nc2c1]

The title compound was prepared from 3,4-diamino-N-(cycloheptylmethyl)benzamide (Example 1.3 step 1) and 3-hydroxy-2-phenyl-propanoic acid analogously to Example 1.3 step 2.

LC-MS (Method A): Rt 2.46 min; MS m/z 392.4=[M+H]+

1H NMR (500 MHz, DMSO-d6) δ 12.56-12.39 (m, 1H), 8.40-8.28 (m, 1H), 8.19-7.89 (m, 1H), 7.70 -7.63 (m, 1H), 7.61-7.40 (m, 1H), 7.39-7.34 (m, 2H), 7.34-7.28 (m, 2H), 7.25-7.20 (m, 1H), 5.00 (br s, 1H), 4.35 (t, J=7.2 Hz, 1H), 4.24-4.17 (m, 1H), 3.98-3.91 (m, 1H), 3.10 (t, J=6.3 Hz, 2H), 1.82 -1.68 (m, 3H), 1.67-1.59 (m, 2H), 1.57-1.34 (m, 6H), 1.23-1.12 (m, 2H).

### Example 1.4

### p N-(Cyclohexylmethyl)-2-[(3-hydroxyphenyl)methyl]-3H-benzimidazole-5-carboxamide

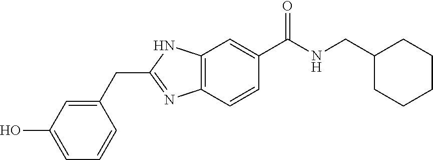 [SMILES: O=C(NCC1CCCCC1)c1ccc2nc(Cc3cccc(O)c3)[nH]c2c1]

Step 1: Methyl 2[(3-benzyloxyphenyl)methyl]-1H-benzimidazole-5-carboxylate

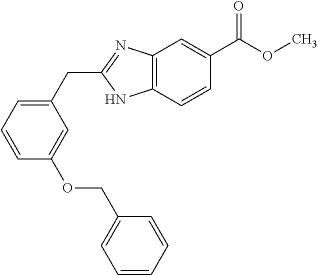 [SMILES: COC(=O)c1ccc2[nH]c(Cc3cccc(OCc4ccccc4)c3)nc2c1]

A solution of 2-(3-benzyloxyphenyl)acetic acid (2.01 g, 8.3 mmol), HATU (3.16 g, 8.3 mmol), and DIPEA (3.19 mL, 18.27 mmol) in DMF (50 mL) was stirred at room temperature for 45 mins and then treated with methyl 3,4-diaminobenzoate (1.38 g, 8.3 mmol). The reaction mixture was stirred at room temperature for 18 h. The resulting mixture was concentrated in vacuo and the residue was partitioned between with sat. NaHCO₃ (100 mL) and EtOAc (125 mL). The organic layer was separated, washed with water (2×75 mL), brine (2×75 mL), dried over Na₂SO₄ and concentrated in vacuo. The crude material was triturated with MeOH (40 mL) and the solid was filtered and dried in a vacuum oven at 40° C. for 3 h. The resulting solid was suspended in AcOH (25 mL) and stirred at 70° C. for 6 h. The mixture was concentrated in vacuo and the residue was partitioned between sat. NaHCO₃ (100 mL) and EtOAc (125 mL). The organic layer was washed with water (2×75 mL), brine (2×75 mL), dried over Na₂SO₄ and concentrated in vacuo to afford the title compound as an off-white powder.

LC-MS (Method E): Rt 1.05 min; MS m/z 373.0=[M+H]+

1H NMR (500 MHz, DMSO-d6) δ 8.10 (d, J=0.9 Hz, 1H), 7.79 (dd, J=8.4, 1.6 Hz, 1H), 7.56 (d, J=8.4 Hz, 1H), 7.44-7.40 (m, 2H), 7.38-7.33 (m, 2H), 7.32-7.28 (m, 1H), 7.24 (t, J=7.9 Hz, 1H), 7.03-7.00 (m, 1H), 6.93-6.87 (m, 2H), 5.07 (s, 2H), 4.19 (s, 2H), 3.85 (s, 3H).

Step 2: Methyl 2[(3-hydroxyphenyl)methyl]-1H-benzimidazole-5-carboxylate

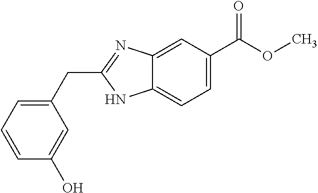 [SMILES: COC(=O)c1ccc2[nH]c(Cc3cccc(O)c3)nc2c1]

To a suspension of methyl 2[(3-benzyloxyphenyl)methyl]-1H-benzimidazole-5-carboxylate (step 1) (95%, 700 mg, 1.79 mmol) in EtOH (20 mL) was added 10% Pd-C (10%, 150 mg, 0.14 mmol). The reaction mixture was placed under a hydrogen atmosphere and stirred at room temperature for 6 h. The resulting mixture was filtered through a plug of Celite® (filter material) washing through with EtOH (45 mL)). The filtrate was concentrated in vacuo to afford the title compound as a pale orange/brown solid.

LC-MS (Method E): Rt 0.83 min; MS m/z 283.1=[M+H]+

1H NMR (500 MHz, DMSO-d6) δ 8.09 (s, 1H), 7.78 (dd, J=8.4, 1.6 Hz, 1H), 7.55 (d, J=8.4 Hz, 1H), 7.10 (t, J=7.8 Hz, 1H), 6.74 (d, J=7.7 Hz, 1H), 6.72-6.69 (m, 1H), 6.62 (dd, J=8.0, 1.8 Hz, 1H), 4.12 (s, 2H), 3.85 (s, 3H). Step 3: 2[(3-Hydroxyphenyl)methyl]-1H-benzimidazole-5-carboxylic acid

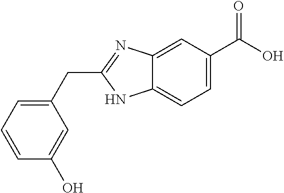 [SMILES: O=C(O)c1ccc2[nH]c(Cc3cccc(O)c3)nc2c1]

2M aq. LiOH solution (2.53 mL, 5.06 mmol) was added to a solution of methyl 24(3-hydroxyphenyl)methyl1-1H-benzimidazole-5-carboxylate (step 2) (95%, 501 mg, 1.69 mmol) in THF (8 mL) and the reaction mixture was stirred at 50° C. for 4 h. The volatile organics were removed in vacuo then the resulting aqueous mixture was acidified to pH 4. The mixture was extracted with 3:1 chloroform:IPA (3×30 mL) and the combined organic extracts were dried over Na₂SO₄ and concentrated in vacuo to afford the title compound as a pale orange powder.

LC-MS (Method E): Rt 0.73 min; MS m/z 269.1=[M+H]+

1H NMR (500 MHz, DMSO-d6) δ 13.48-11.55 (m, 2H), 9.37 (s, 1H), 8.10 (d, J=0.8 Hz, 1H), 7.81 (dd, J=8.4, 1.5 Hz, 1H), 7.57 (d, J=8.4 Hz, 1H), 7.12 (t, J=7.8 Hz, 1H), 6.75 (d, J=7.7 Hz, 1H), 6.73-6.70 (m, 1H), 6.64 (dd, J=8.0, 1.8 Hz, 1H), 4.16 (s, 2H).

Step 4: N-(Cyclohexylmethyl)-24(3-hydroxyphenyl)methyll -3H-benzimidazole-5-carboxamide

A solution of 2[(3-hydroxyphenyl)methyl]-3H-benzimidazole-5-carboxylic acid (step 3) (50 mg, 0.19 mmol), EDCI (33 mg, 0.21 mmol), DMAP (46 mg, 0.37 mmol) and HOAt (28 mg, 0.21 mmol) in DMF (1 mL) was stirred for 5 mins then treated with cyclohexylmethanamine (48.5 μL, 0.37 mmol). The resulting mixture was stirred at room temperature for 2 h and concentrated in vacuo. The residue was taken up in EtOAc (5 mL) and the organics were washed with water (3×5 mL), brine (5 mL), dried over Na₂SO₄ and concentrated in vacuo. The crude product was purified by preparative HPLC (basic pH, standard elution method) followed by further purification by preparative HPLC (acidic pH, standard elution method) to afford the title compound as an off-white solid.

LC-MS (Method A): Rt 2.00 min; MS m/z 364.2=[M+H]+

1H NMR (500 MHz, DMSO-d6) δ 12.52-12.34 (m, 1H), 9.33 (s, 1H), 8.39-8.28 (m, 1H), 8.13-7.84 (m, 1H), 7.76-7.60 (m, 1H), 7.60-7.33 (m, 1H), 7.11 (t, J=7.8 Hz, 1H), 6.74 (d, J=7.6 Hz, 1H), 6.71-6.66 (m, 1H), 6.63 (dd, J=8.0, 2.1 Hz, 1H), 4.11 (s, 2H), 3.12 (t, J=6.4 Hz, 2H), 1.77-1.65 (m, 4H), 1.65 -1.51 (m, 2H), 1.28-1.08 (m, 3H), 1.00-0.85 (m, 2H).

### Example 1.5

### 2-(1-Adamantyl)-N-12-[(3-hydroxyphenyl)methyl]-1H-benzimidazol-5-yljacetamide

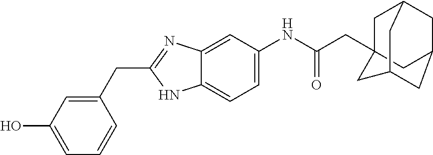 [SMILES: O=C(CC12CC3CC(CC(C3)C1)C2)Nc1ccc2[nH]c(Cc3cccc(O)c3)nc2c1]

Step 1: 2[(3-Benzyloxyphenyl)methyl]-5-nitro-1H-benzimidazole

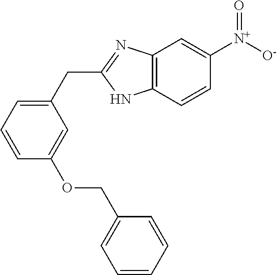 [SMILES: O=[N+]([O-])c1ccc2[nH]c(Cc3cccc(OCc4ccccc4)c3)nc2c1]

A solution of 2-(3-benzyloxyphenyl)acetic acid (2.99 g, 12.34 mmol), HATU (4.69 g, 12.34 mmol) and DIPEA (5.27 mL, 30.17 mmol) in DMF (50 mL) was stirred at room temperature for 45 mins then treated with 4-nitrobenzene-1,2-diamine (2.1 g, 13.71 mmol). The reaction mixture was stirred at room temperature for 60 h then diluted with sat. NaHCO₃ (50 mL) and EtOAc (100 mL). The phases were separated and the organic portion was washed with water (2×50 mL), brine (2×50 mL), dried over Na₂SO₄ and concentrated in vacuo. The residue was dissolved in acetic acid (40 mL) and stirred at 70° C. for 2.5 h. Additional AcOH (20 mL) was added and stirring continued at 70° C. for 1 h. The reaction mixture was concentrated in vacuo and the residue was partitioned between sat. NaHCO₃ (50 mL) and EtOAc (100 mL). The organic portion was separated, washed with water (50 mL), brine (50 mL), dried over Na₂SO₄ and concentrated in vacuo. Purification of the crude product by chromatography on silica eluting with 20-75% EtOAc in heptanes followed by C18 reverse phase chromatography eluting with 10-100% MeCN in water (0.1% formic acid) afforded the title compound as a bright orange solid.

LC-MS (Method E): Rt 1.17 min; MS m/z 360.0=[M+H]+

1H NMR (500 MHz, DMSO-d6) δ 12.94 (br s, 1H), 8.41 (br s, 1H), 8.07 (d, J=8.6 Hz, 1H), 7.65 (br s, 1H), 7.47-7.19 (m, 6H), 7.01 (s, 1H), 6.94-6.88 (m, 2H), 5.07 (s, 2H), 4.23 (s, 2H). Step 2: 34(5-Amino-1H-benzimidazol-2-yl)methyllphenol

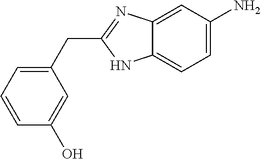 [SMILES: Nc1ccc2[nH]c(Cc3cccc(O)c3)nc2c1]

To a suspension of 2[(3-benzyloxyphenyl)methyl]-5-nitro-1H-benzimidazole (step 1) (89%, 250 mg, 0.62 mmol) in EtOH (30 mL) was added 10% Pd-C (10%, 66 mg, 0.06 mmol). The reaction was placed under a hydrogen atmosphere and stirred at room temperature for 6 h. The resulting mixture was passed through a plug of Celite® (filter material) and washed through with EtOH (˜35 mL). The filtrate was concentrated in vacuo then azeotroped with Et₂O (3×15 mL) to afford the title compound as a grey powder.

LC-MS (Method F): Rt 1.19 min; MS m/z 240.2=[M+H]+

1H NMR (500 MHz, DMSO-d6) δ 11.61 (br. s, 1H), 9.28 (br. s, 1H), 7.22-7.04 (m, 2H), 6.75-6.37 (m, 5H), 4.74 (br. s, 2H), 3.95 (s, 2H).

Step 3: 2-(1-Adamantyl)-N[2-[(3-hydroxyphenyl)methyl]-1H-benzimidazol-5-yl] acetamide

A solution of HATU (119 mg, 0.31 mmol), 2-(1-adamantyl)acetic acid (55 mg, 0.28 mmol) and DIPEA (109 μL, 0.63 mmol) in DMF (2 mL) was stirred for 30 mins at room temperature then treated with 34(5-amino-1H-benzimidazol-2-yl)methyllphenol (step 2) (80%, 85 mg, 0.28 mmol). The reaction mixture was stirred at room temperature for 1 h and concentrated in vacuo. The residue was taken up in EtOAc (5 mL) and washed with water (3×5 mL), brine (5 mL), dried over Na₂SO₄ and concentrated in vacuo. The crude product was purified by preparative HPLC (basic pH, standard elution method) to afford the title compound as an off- white powder.

LC-MS (Method A): Rt 2.28 min; MS m/z 416.3=[M+H]+

1H NMR (500 MHz, DMSO-d6) δ 12.14-12.03 (m, 1H), 9.73-9.57 (m, 1H), 9.31 (s, 1H), 7.99-7.81 (m, 1H), 7.46-7.04 (m, 3H), 6.75-6.56 (m, 3H), 4.03 (s, 2H), 2.05 (s, 2H), 1.93 (s, 3H), 1.71-1.52 (m, 12H).

### Example 1.5.1

### N-[2-[(3-hydroxyphenyl)methyl]-1H-benzimidazol-5-yl]-2-(1-methylcyclohexyl) acetamide

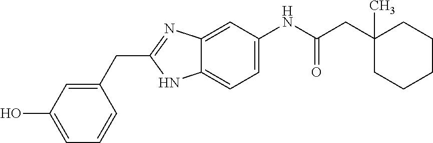 [SMILES: CC1(CC(=O)Nc2ccc3[nH]c(Cc4cccc(O)c4)nc3c2)CCCCC1]

The title compound was prepared from 34(5-amino-1H-benzimidazol-2-yl)methyllphenol (Example 1.5 step 2) and 2-(1-methylcyclohexyl)acetic acid analogously to Example 1.5 step 3. LC-MS (Method A): Rt 2.11 min; MS m/z 378.3=[M+H]+

1H NMR (500 MHz, DMSO-d6) δ 12.07 (br s, 1H), 9.69 (s, 1H), 9.39 (br s, 1H), 7.90 (s, 1H), 7.36 (d, J=8.2 Hz, 1H), 7.25-7.12 (m, 1H), 7.08 (t, J=7.8 Hz, 1H), 6.71 (d, J=7.6 Hz, 1H), 6.69-6.66 (m, 1H), 6.60 dd, J=8.0, 1.9 Hz, 1H), 4.03 (s, 2H), 2.21 (s, 2H), 1.53-1.38 (m, 7H), 1.35-1.26 (m, 3H), 1.03 (s, 3H).

### Example 1.5.2

### 2-Cycloheptyl-N-[2- [(3-hydroxyphenyl)methyl]-1H-benzimidazol-5-yl]acetamide

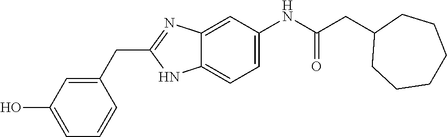 [SMILES: O=C(CC1CCCCCC1)Nc1ccc2[nH]c(Cc3cccc(O)c3)nc2c1]

The title compound was prepared from 34(5-amino-1H-benzimidazol-2-yl)methyllphenol (Example 1.5 step 2) and 2-cycloheptylacetic acid analogously to Example 1.5 step 3.

LC-MS (Method A): Rt 2.11 min; MS m/z 378.3=[M+H]+

1H NMR (500 MHz, DMSO-d6) δ 12.17-12.04 (m, 1H), 9.80-9.68 (m, 1H), 9.30 (s, 1H), 7.96-7.83 (m, 1H), 7.45-7.10 (m, 2H), 7.08 (t, J=7.8 Hz, 1H), 6.71 (d, J=7.6 Hz, 1H), 6.69-6.65 (m, 1H), 6.63-6.58 (m, 1H), 4.05-4.00 (m, 2H), 2.24-2.13 (m, 2H), 2.06-1.92 (m, 1H), 1.75-1.65 (m, 2H), 1.65-1.51 (m, 4H), 1.51-1.34 (m, 4H), 1.27-1.16 (m, 2H).

### Example 1.5.3

### 2-Cyclohexyl-N-12- [(3-hydroxyphenyl)methyl]-1H-benzimidazol-5-yljacetamide

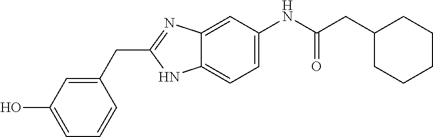 [SMILES: O=C(CC1CCCCC1)Nc1ccc2[nH]c(Cc3cccc(O)c3)nc2c1]

The title compound was prepared from 34(5-amino-1H-benzimidazol-2-yl)methyllphenol (Example 1.5 step 2) and 2-cyclohexylacetic acid analogously to Example 1.5 step 3.

LC-MS (Method A): Rt 1.89 min; MS m/z 364.3=[M+H]+

1H NMR (500 MHz, DMSO-d6) δ 12.12-12.04 (m, 1H), 9.80-9.67 (m, 1H), 9.30 (s, 1H), 7.96-7.83 (m, 1H), 7.45-7.04 (m, 3H), 6.72 (d, J=7.5 Hz, 1H), 6.69-6.65 (m, 1H), 6.61 (dd, J=8.0, 1.8 Hz, 1H), 4.05-4.01 (m, 2H), 2.20-2.15 (m, 2H), 1.86-1.55 (m, 6H), 1.32-1.06 (m, 3H), 1.06-0.91 (m, 2H).

### Example 1.5.4

### 2-(1-Adamantyl)-N-(2-benzyl-1H-benzimidazol-5-yl)acetamide

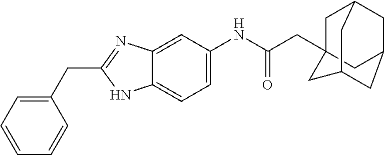 [SMILES: O=C(CC12CC3CC(CC(C3)C1)C2)Nc1ccc2[nH]c(Cc3ccccc3)nc2c1]

The title compound was prepared from 2-benzyl-1H-benzimidazol-5-amine (Intermediate A) and 2-(1-adamantyl)acetic acid analogously to Example 1.5 step 3 .

LC-MS (Method C): Rt 3.51 min; MS m/z 400.3=[M+H]+

1H NMR (500 MHz, DMSO-d6) δ 12.10 (s, 1H), 9.84-9.40 (m, 1H), 8.02-7.78 (m, 1H), 7.48-7.05 (m, 7H), 4.13 (s, 2H), 2.04 (s, 2H), 1.93 (s, 3H), 1.75-1.48 (m, 12H).

### Example 1.6

### N-(2-Benzyl-1H-benzimidazol-5-yl)-2-(2-hydroxy-2-adamantypacetamide

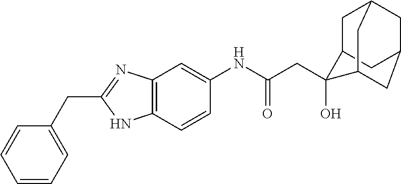 [SMILES: O=C(CC1(O)C2CC3CC(C2)CC1C3)Nc1ccc2[nH]c(Cc3ccccc3)nc2c1]

Step 1: 2-(2-Hydroxy-2-adamantyl)acetic acid

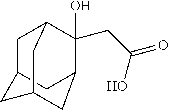 [SMILES: O=C(O)CC1(O)C2CC3CC(C2)CC1C3]

To a solution of diisopropylamine (350 μL, 2.5 mmol) in THF (4 mL) at −78° C. was added n-BuLi (1.6M in hexanes) (1.56 mL, 2.5 mmol). The mixture was warmed to 0° C., stirred for 30 mins then re-cooled to -78° C. Acetic acid (95 μL, 1.66 mmol) added followed by n-BuLi (1.6M in hexanes) (1.04 mL, 1.66 mmol). The solution was warmed to 0° C., stirred for 30 mins then re-cooled to -78° C. and adamantan-2-one (751 mg, 5.0 mmol) in THF (1.5 mL) was added. The resulting solution was allowed to warm to room temperature and stirred for 1 h. The reaction was quenched by addition of sat. NH₄Cl solution (1 mL). The mixture was diluted with diethyl ether (10 mL) and washed with 2M NaOH solution (10 mL). The ether layer was separated and discarded. The aqueous layer was acidified to pH 2 using 2M HC1 solution and extracted with EtOAc (10 mL). The EtOAc solution was washed with water (10 mL), brine (10 mL), dried over Na₂SO₄ and concentrated in vacuo to afford the title compound as a colourless powder.

LC-MS (Method E): Rt 0.98 min; MS m/z 209.0=

1H NMR (250 MHz, DMSO-d6) δ 11.99 (br s, 1H), 4.48 (br s, 1H), 2.55 (s, 2H), 2.20 (d, J=12.5 Hz, 2H), 1.92-1.57 (m, 10H), 1.40 (d, J=12.2 Hz, 2H).

Step 2: N-(2-Benzyl-1H-benzimidazol-5-yl)-2-(2-hydroxy-2-adamantypacetamide

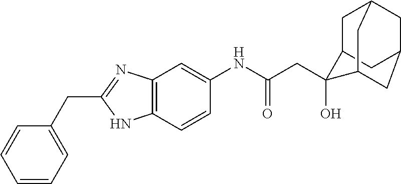 [SMILES: O=C(CC1(O)C2CC3CC(C2)CC1C3)Nc1ccc2[nH]c(Cc3ccccc3)nc2c1]

To a solution of 2-(2-hydroxy-2-adamantyl)acetic acid (step 1) (120 mg, 0.57 mmol), DIPEA (209 μL, 1.2 mmol) and 2-benzyl-1H-benzimidazol-5-amine (Intermediate A) (127 mg, 0.57 mmol) in DMF (3 mL) was added HATU (239 mg, 0.63 mmol) and the mixture stirred at room temperature for 1 h. The resulting mixture was diluted with EtOAc (10 mL) and washed with water (10 mL), brine (10 mL), dried over Na₂SO₄ and concentrated in vacuo. The crude residue was triturated with a mixture MeCN/MeOH/water (1:1:1) and the resulting solids collected by filtration. The solids were washed with MeCN/water (1:1) and ether then dried under a flow of nitrogen to afford the title compound as an off-white powder.

LC-MS (Method A): Rt 2.20 min; MS m/z 416.3=[M+H]+

1H NMR (250 MHz, DMSO-d6) δ 12.23-12.11 (m, 1H), 10.01-9.89 (m, 1H), 7.96-7.80 (m, 1H), 7.46 -7.10 (m, 7H), 5.13-5.03 (m, 1H), 4.14 (s, 2H), 2.69 (s, 2H), 2.28-2.19 (m, 2H), 1.92 (d, J=12.4 Hz, 2H), 1.80 (s, 1H), 1.75-1.61 (m, 7H), 1.42 (d, J=11.9 Hz, 2H).

### Example 1.7

### 2-(2-Adamantyl)-N-12-[(3-methoxyphenyl)methyl]-1H-benzimidazol-5-yljacetamide

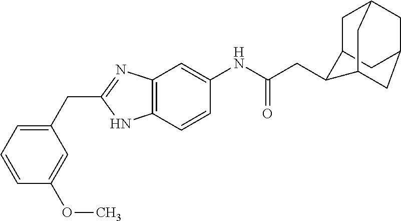 [SMILES: COc1cccc(Cc2nc3cc(NC(=O)CC4C5CC6CC(C5)CC4C6)ccc3[nH]2)c1]

2-(3-Methoxyphenyl)acetic acid (83 mg, 0.50 mmol), HATU (210 mg, 0.55 mmol) and DIPEA (0.18 mL, 1.00 mmol) were dissolved in DMF (1.5 mL) and the mixture was stirred at room temperature for 30 mins. A solution of 2-(2-adamantyl)-N-(3,4-diaminophenyl)acetamide (Intermediate B) (150 mg, 0.50 mmol) in DMF (1 mL) was added and the reaction mixture was stirred at room temperature overnight. Additional 2-(3-methoxyphenyl)acetic acid (42 mg, 0.25 mmol) , HATU (105 mg, 0.28 mmol) and DIPEA (0.09 mL, 0.5 mmol) were stirred in DMF (0.5 mL) for 10 mins then added to the main reaction mixture. After stirring at room temperature for 4.5 h, the resulting mixture was diluted with EtOAc (10 mL) and saturated aqueous sodium bicarbonate solution (10 mL). The phases were separated and the organic portion was washed with water (2×20 mL), brine (10 mL) and concentrated in vacuo. Purification of the crude material by chromatography on silica eluting with a gradient of 0-100% EtOAc in heptanes afforded a brown oil. The oil was triturated in EtOAc to yield an off-white solid. The solid was suspended in acetic acid (1 mL) and stirred at 60° C. for 5 h. After cooling to room temperature, the mixture was concentrated in vacuo and the residue was partitioned between EtOAc (5 mL) and saturated aqueous sodium bicarbonate solution (5 mL). The layers were separated and the organic layer was washed with saturated aqueous sodium bicarbonate solution (3×5 mL), passed through a hydrophobic frit and concentrated in vacuo. The resulting oil was azeotroped thrice with MeCN to afford the title compound as a colourless solid.

LC-MS (Method A): Rt 2.83 min; MS m/z 430=[M+H]+

1H NMR (500 MHz, DMSO-d6) δ 12.12 (s, 1H), 9.91-9.70 (m, 1H), 8.06-7.79 (m, 1H), 7.49-7.05 (m, 3H), 6.97-6.85 (m, 2H), 6.84-6.74 (m, 1H), 4.10 (s, 2H), 3.73 (s, 3H), 2.45 (d, J=7.5 Hz, 2H), 2.28-2.21 (m, 1H), 1.99-1.65 (m, 12H), 1.56-1.49 (m, 2H).

The compounds of the following tabulated Examples (Table Ex1.7) were prepared analogously to Example 1.7 from 2-(2-adamantyl)-N-(3,4-diaminophenyl)acetamide (Intermediate B) and the appropriate commercially available acid.

| Ex. | Structure and Name | Retention Time, [M + H]+, 1H NMR |
|---|---|---|
| 1.7.1 | 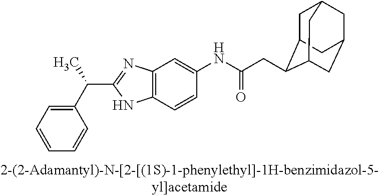 [SMILES: C[C@@H](c1ccccc1)c1nc2cc(NC(=O)CC3C4CC5CC(C4)CC3C5)ccc2[nH]1] | LC-MS (Method A): Rt 2.65 min; MS m/z 414.3 = [M + H]+ 1H NMR (500 MHz, DMSO-d6) δ 12.11 − 11.96 (m, 1H), 9.91 − 9.69 (m, 1H), 8.02 − 7.77 (m, 1H), 7.51 − 7.03 (m, 7H), 4.41 − 4.23 (m, 1H), 2.47 − 2.41 (obscured m, 2H), 2.27 − 2.19 (m, 1H), 1.99 − 1.63 (m, 15H), 1.57 − 1.46 (m, 2H). |
| 1.7.2 | 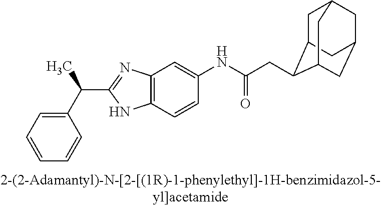 [SMILES: C[C@H](c1ccccc1)c1nc2cc(NC(=O)CC3C4CC5CC(C4)CC3C5)ccc2[nH]1] | LC-MS (Method A): Rt 2.63 min; MS m/z 414.3 = [M + H]+ 1H NMR (500 MHz, DMSO-d6) δ 12.10 − 12.00 (m, 1H), 9.85 − 9.72 (m, 1H), 7.95 − 7.83 (m, 1H), 7.45 − 7.27 (m, 5H), 7.25 − 7.17 (m, 1H), 7.15 − 7.10 (m, 1H), 4.33 (q, J = 6.9 Hz, 1H), 2.44 (d, J = 7.6 Hz, 2H), 2.27 − 2.19 (m, 1H), 1.97 − 1.91 (m, 2H), 1.88 − 1.64 (m, 13H), 1.55 − 1.48 (m, 2H). |

### Example 1.7.3

### tert-Butyl N-[[5-[[2-(1-adamantyl)acetyl]amino]-1H-benzimidazol-2-yl]methyl]carbamate

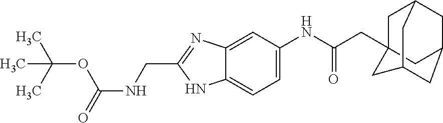 [SMILES: CC(C)(C)OC(=O)NCc1nc2cc(NC(=O)CC34CC5CC(CC(C5)C3)C4)ccc2[nH]1]

The title compound was prepared from 2-(1-adamantyl)-N-(3,4-diaminophenyl)acetamide (Intermediate C) and 2-(tert-butoxycarbonylamino)acetic acid analogously to Example 1.7.

LC-MS (Method A): Rt 2.69 min; MS m/z 439.3=[M+H]+

1H NMR (500 MHz, DMSO-d6) δ 12.02 (br. s, 1H), 9.67 (s, 1H), 7.94 (s, 1H), 7.46-7.26 (m, 2H), 7.19 (s, 1H), 4.31 (d, J=5.8 Hz, 2H), 2.05 (s, 2H), 1.94 (s, 3H), 1.71-1.57 (m, 12H), 1.41 (s, 9H).

### Example 1.7.4

### 2-(1-Adamantyl)-N-12-[(2-methoxy-3-pyridyl)methyl]-1H-benzimidazol-5-yljacetamide

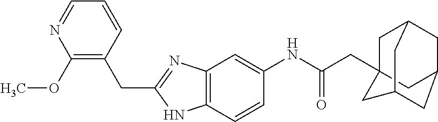 [SMILES: COc1ncccc1Cc1nc2cc(NC(=O)CC34CC5CC(CC(C5)C3)C4)ccc2[nH]1]

The title compound was prepared from 2-(1-adamantyl)-N-(3,4-diaminophenyl)acetamide (Intermediate C) and 2-(2-methoxy-3-pyridyl)acetic acid analogously to Example 1.7.

LC-MS (Method A): Rt 2.37 min; MS m/z 431.3=[M+H]+

1H NMR (500 MHz, DMSO-d6) δ 12.03 (br s, 1H), 9.73-9.59 (m, 1H), 8.09 (dd, J=5.0, 1.8 Hz, 1H), 8.01-7.79 (m, 1H), 7.55 (dd, J=7.2, 1.7 Hz, 1H), 7.41-7.07 (m, 2H), 6.96 (dd, J=7.2, 5.0 Hz, 1H), 4.09 (s, 2H), 3.87 (s, 3H), 2.05 (s, 2H), 1.94 (s, 3H), 1.69-1.58 (m, 12H).

### Example 1.8

### 2-(2-Adamantyl)-N-12-[(3-hydroxyphenyl)methyl]-1H-benzimidazol-5-yljacetamide

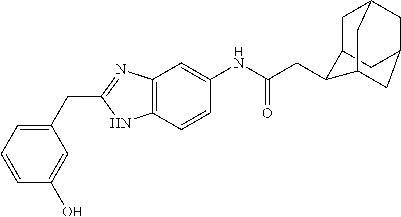 [SMILES: O=C(CC1C2CC3CC(C2)CC1C3)Nc1ccc2[nH]c(Cc3cccc(O)c3)nc2c1]

1M BBr₃ in DCM (0.20 mL, 0.20 mmol) was added dropwise to an ice cold solution of 2-(2-adamantyl)-N-[2-[(3-methoxyphenyl)methyl]-1H-benzimidazol-5-yl]acetamide (Example 1.7) (44 mg, 0.10 mmol) in DCM (3 mL). The mixture was stirred in the ice bath for 5 mins and then at room temperature overnight. The reaction mixture was re-cooled in the ice bath and treated with additional 1M BBr₃ in DCM (0.10 mL, 0.10 mmol) and stirring continued at room temperature for 6 h. Water (5 mL) was added slowly to the stirring reaction mixture. The majority of aqueous layer was carefully removed with a pipette. The remaining DCM/aqueous suspension was filtered under vacuum to afford a white solid. The solid was dissolved in MeOH and purified by C18 reverse phase chromatography eluting withl0-100% MeCN in water (+0.1% formic acid) to afford the title compound as a colourless solid.

LC-MS (Method A): Rt 2.57 min; MS m/z 416=[M+H]+

1H NMR (500 MHz, DMSO-d6) δ 12.10 (s, 1H), 9.82 (s, 1H), 9.33 (s, 1H), 7.92 (s, 1H), 7.48-7.04 (m, 3H), 6.74-6.70 (m, 1H), 6.69-6.66 (m, 1H), 6.62-6.58 (m, 1H), 4.03 (s, 2H), 2.45 (d, J=7.7 Hz, 2H), 2.26-2.19 (m, 1H), 1.99-1.91 (m, 2H), 1.88-1.63 (m, 10H), 1.56-1.46 (m, 2H).

### Example 1.9

### 2-tert-Butyl-N-[(5-chloro-2-hydroxy-phenyl)methyl]-1H-benzimidazole-5-carboxamide

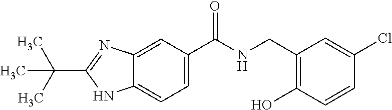 [SMILES: CC(C)(C)c1nc2cc(C(=O)NCc3cc(Cl)ccc3O)ccc2[nH]1]

Step 1: Methyl 2-tert-butyl-1H-benzimidazole-5-carboxylate

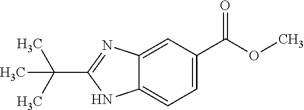 [SMILES: COC(=O)c1ccc2[nH]c(C(C)(C)C)nc2c1]

To a solution of methyl 3,4-diaminobenzoate (200 mg, 1.2 mmol), 2,2-dimethylpropanoic acid (148 mg, 1.44 mmol) and DIPEA (0.25 mL, 1.44 mmol) in DMF (7 mL) was added HATU (503 mg, 1.32 mmol) and the mixture stirred at room temperature for 1 h. Additional 2,2-dimethylpropanoic acid (148 mg, 1.44 mmol), DIPEA (0.25 mL, 1.44 mmol) and HATU (503 mg, 1.32 mmol) were added and stirring was continued overnight. The resulting mixture was diluted with EtOAc (15 mL) and washed water (10 mL) and brine (10 mL), dried over Na₂SO₄ and concentrated in vacuo The crude residue was dissolved in acetic acid (7 mL) and heated at 60° C. for 2 h and then at 70° C. for 2 h. After cooling to room temperature, the mixture was concentrated in vacuo. The residue was dissolved in EtOAc (10 mL) then washed with sat. NaHCO₃ solution (10 mL), brine (10 mL), dried over Na₂SO₄ and concentrated in vacuo. The crude residue was purified by chromatography on silica eluting with 30-100% EtOAc in heptanes to afford the title compound as a pale brown glassy solid.

LC-MS (Method E): Rt 0.80 min; MS m/z 233.3=[M+H]+

1H NMR (500 MHz,Chloroform-d) δ 10.01 (br s, 1H), 8.48-8.10 (m, 1H), 7.96-7.90 (m, 1H), 7.79-7.32 (m, 1H), 3.92 (s, 3H), 1.52 (s, 9H).

Step 2: 2-tert-Butyl-1H-benzimidazole-5-carboxylic acid

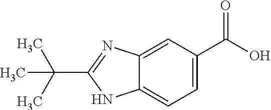 [SMILES: CC(C)(C)c1nc2cc(C(=O)O)ccc2[nH]1]

To a solution of methyl 2-tert-butyl-1H-benzimidazole-5-carboxylate (step 1) (213 mg, 0.92 mmol) in MeOH (1.5 mL), THF (1.5 mL) and water (1.5 mL) was added LiOH (26 mg, 1.1 mmol) and the mixture stirred at room temperature overnight. Additional LiOH (26 mg, 1.1 mmol was added and the reaction stirred at room temperature for 8 h. Further LiOH (79 mg, 3.3 mmol) was added and the mixture was stirred for 30 h. The reaction was quenched by addition of 1M HC1 solution to pH 4. The aqueous mixture was extracted with EtOAc (10 mL), CHC13/IPA (1:1) (10 mL) and the combined organic extracts were dried over Na₂SO₄ and concentrated in vacuo to afford the title compound as pale pink powder.

LC-MS (Method E): Rt 0.77 min; MS m/z 219.0=[M+H]+

Step 3: (5-Chloro-2-methoxy-phenyl)methanamine

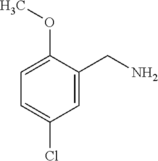

A solution of 5-chloro-2-methoxy-benzonitrile (2.0 g, 11.93 mmol) in THF (30 mL) was added dropwise to a solution of lithium aluminium hydride (2.4M in THF, 7.46 mL, 17.9 mmol) in THF (22.5 mL) at 0° C. Once addition was complete the mixture was warmed to room temperature and stirred for 1 h. The reaction was quenched by slow addition of 1M NaOH solution (10 mL) at 0° C. The resulting mixture was diluted with EtOAc (50 mL) and 1M NaOH solution (50 mL) and filtered to remove the suspended solids. The organic layer was separated, washed with brine, dried over Na₂SO₄ and concentrated in vacuo to afford the title compound as a yellow oil.

LC-MS (Method E): Rt 0.72 min; MS m/z 172.0, 174.0=[M+H]+

1H NMR (250 MHz, Chloroform-d) δ 7.23-7.13 (m, 2H), 6.77 (d, J=8.5 Hz, 1H), 3.83 (s, 3H), 3.78 (s, 2H).

Step 4: 2-tert-Butyl-N- [(5-chloro-2-methoxy-phenyl)methyl]-1H-benzimidazole-5-carboxamide

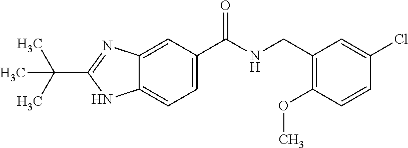 [SMILES: COc1ccc(Cl)cc1CNC(=O)c1ccc2[nH]c(C(C)(C)C)nc2c1]

To a solution of 2-teat-butyl-1H-benzimidazole-5-carboxylic acid (step 2)(80 mg, 0.37 mmol) in DMF (1 mL) was added DIPEA (57 mg, 0.44 mmol) and HATU (167 mg, 0.44 mmol) followed by a solution of (5-chloro-2-methoxy-phenyl)methanamine (step 3) (90%, 84 mg, 0.44 mmol) in DMF (1 mL) and the mixture stirred at room temperature for 1 h. The resulting mixture was diluted with EtOAc (10 mL) and washed with water (10 mL), brine (10 mL), dried over Na₂SO₄ and concentrated in vacuo. The crude residue was purified by chromatography on silica eluting with 50-100% EtOAc in heptanes to afford the title compound as pale pink glass.

LC-MS (Method E): Rt 1.02 min; MS m/z 372.0/374.0=[M+H]+

1H NMR (250 MHz, Methanol-d4) δ 8.08 (br s, 1H), 7.75 (dd, J=8.4, 1.6 Hz, 1H), 7.63-7.52 (m, 1H), 7.27-7.18 (m, 2H), 6.97 (d, J=8.4 Hz, 1H), 4.57 (s, 2H), 3.89 (s, 3H), 1.49 (s, 9H).

Step 5: 2-tert-Butyl-N- [(5-chloro-2-hydroxy-phenyl)methyl]-1H-benzimidazole -5-carboxamide

To solution of 2-tert-butyl-N-R5-chloro-2-methoxy-phenyl)methyll -1H-benzimidazole-5-carboxamide (step 4) (94%, 124 mg, 0.31 mmol) in DCM (1 mL) at 0° C. was added 1M BBr₃ in DCM (0.47 mL, 0.47 mmol) and the reaction mixture was allowed to stir at room temperature overnight. The reaction was quenched by dropwise addition of sat. NaHCO₃ solution (5 mL) and the resulting mixture was diluted with EtOAc (10 mL) and sat. NaHCO₃ solution (5 mL). The organic portion was separated, washed with brine (10 mL), dried over Na₂SO₄ and concentrated in vacuo. Purification of the crude residue by preparative HPLC (basic pH, standard elution method) afforded the title compound as a colourless powder.

LC-MS (Method A): Rt 1.77 min; MS m/z 358.1/360.1=[M+H]+

1H NMR (500 MHz, Methanol-d4) δ 8.08 (s, 1H), 7.74 (dd, J=8.4, 1.6 Hz, 1H), 7.57 (d, J=8.3 Hz, 1H), 7.21 (d, J=2.6 Hz, 1H), 7.08 (dd, J=8.6, 2.6 Hz, 1H), 6.79 (d, J=8.6 Hz, 1H), 4.54 (s, 2H), 1.49 (s, 9H).

### Example 1.10

### tert-Butyl N-[[5-(cycloheptylmethylcarbamoyl)-1H-benzimidazol-2-yl]methyl] carbamate

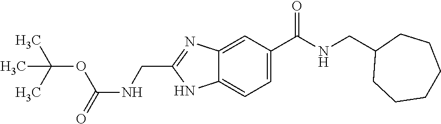 [SMILES: CC(C)(C)OC(=O)NCc1nc2cc(C(=O)NCC3CCCCCC3)ccc2[nH]1]

Step 1: Methyl 2-[(tert-butoxycarbonylamino)methyl]-1H-benzimidazole-5-carboxylate

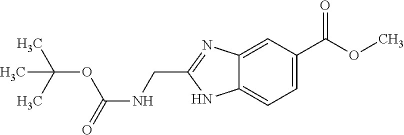 [SMILES: COC(=O)c1ccc2[nH]c(CNC(=O)OC(C)(C)C)nc2c1]

To a stirred solution of 2-(tert-butoxycarbonylamino)acetic acid (3.45 g, 19.70 mmol) in DMF (100 mL) was added HATU (8.24 g, 21.67 mmol) and DIPEA (3.78 mL, 21.67 mmol) and the mixture stirred for 10 mins at room temperature. Methyl 3,4-diaminobenzoate (3.60 g, 21.67 mmol) was added and the mixture stirred for 17 h. The resulting mixture was diluted with water (100 mL) and extracted with EtOAc (6×100 mL). The combined organic extracts were washed with brine (3×100 mL), dried over Na₂SO₄ and concentrated in vacuo. The intermediate was dissolved in acetic acid (50 mL) and heated to 60° C. for 80 mins. The resulting mixture was concentrated in vacuo and the residue was dissolved in EtOAc (50 mL). The organics were washed with saturated aqueous sodium bicarbonate solution (2×30 mL), brine (2×30 mL), dried over Na₂SO₄ and concentrated in vacuo. Purification by chromatography on silica eluting with 50-100% EtOAc in heptanes afforded the title compound.

LC-MS (Method E): Rt 0.91 min; MS m/z 306.1=[M+H]+

1H NMR (250 MHz, DMSO-d6) δ 12.54 (br s, 1H), 8.12 (d, J=1.1 Hz, 1H), 7.80 (dd, J=8.4, 1.6 Hz, 1H), 7.58 (d, J=8.5 Hz, 1H), 7.47 (t, J=5.4 Hz, 1H), 4.38 (d, J=5.9 Hz, 2H), 3.86 (s, 3H), 1.41 (s, 9H).

Step 2: 2-[(tert-Butoxycarbonylamino)methyl]-1H-benzimidazole-5-carboxylic acid

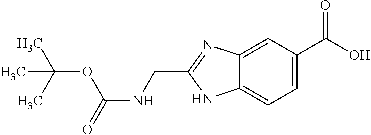 [SMILES: CC(C)(C)OC(=O)NCc1nc2cc(C(=O)O)ccc2[nH]1]

Methyl 2-Rtert-butoxycarbonylamino)methyll-1H-benzimidazole-5-carboxylate (step 1) (500 mg, 1.64 mmol) was added to solution of LiOH (39 mg, 1.64 mmol) in a mixture of MeOH (5 mL), THF (5 mL) and water (5 mL) and stirred at 50° C. for 1 h 40 mins. Additional LiOH (39 mg, 1.64 mmol) was added and stirring continued at 50° C. for 3 h 35 mins. Further LiOH (117 mg mg, 4.91 mmol) was added and the temperature increased to 60° C. and the reaction was allowed to continue overnight. Additional LiOH (390 mg, 16.4 mmol) was added and stirring continued for 6 h at 60° C. A final portion of LiOH (390 mg, 16.4 mmol) was added and the mixture stirred and heated at 60° C. for an additional 23 h. The resulting mixture was acidified to pH 4 with 1M HC1 and extracted with EtOAc (5×10 mL). The combined organic extracts were concentrated in vacuo to afford the title compound as a colourless powder.

LC-MS (Method E): Rt 0.83 min; MS m/z 292.0=[M+H]+

1H NMR (250 MHz, Methanol-d4) δ 8.47-8.38 (m, 1H), 8.28-8.16 (m, 1H), 7.90-7.81 (m, 1H), 4.80 -4.70 (m, 2H), 1.68-1.07 (m, 9H).

Step 3: tert-Butyl N-[[5-(cycloheptylmethylcarbamoyl)-1H-benzimi dazol-2-yl]methyl]carbamate

To a solution of cycloheptylmethanamine (74 μL, 0.51 mmol) and 2-Rtert-butoxycarbonylamino)methyl1-1H-benzimidazole-5-carboxylic acid (step 2) (85%, 160 mg, 0.47 mmol) in DMF (3 mL) was added HATU (195 mg, 0.51 mmol) and the mixture stirred for 10 mins. DIPEA (90 μL, 0.51 mmol) was added and the reaction mixture stirred at room temperature for 3 h 30 mins. The resulting mixture was diluted with water (5 mL) and extracted with EtOAc (3×5 mL). The combined organic extracts were washed with brine (2×5 mL), dried over Na₂SO₄ and concentrated in vacuo. The residue was redissolved in EtOAc (2 mL), washed with saturated aqueous sodium bicarbonate solution (2×2 mL) and concentrated in vacuo. Purification by C18 reverse phase chromatography eluting with 10-100% water in MeCN (0.1% ammonium hydroxide) afforded the title compound as a colourless solid.

LC-MS (Method A): Rt 2.52 min; MS m/z 401.3=[M+H]+

1H NMR (500 MHz, Methanol-d4) δ 8.03 (s, 1H), 7.71 (dd, J=8.4, 1.3 Hz, 1H), 7.57 (d, J=8.4 Hz, 1H), 4.51 (s, 2H), 3.24 (d, J=7.0 Hz, 2H), 1.91-1.78 (m, 3H), 1.76-1.68 (m, 2H), 1.67-1.59 (m, 2H), 1.59 -1.42 (m, 12H), 1.33-1.23 (m, 3H).

### Example 1.10.1

### 2-Benzyl-N-1(1-methylcyclohexyl)methylj -1H-benzimidazole-5-carboxamide

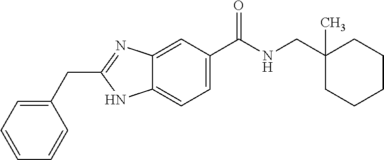 [SMILES: CC1(CNC(=O)c2ccc3[nH]c(Cc4ccccc4)nc3c2)CCCCC1]

The title compound was prepared from 2-benzyl-1H-benzimidazole-5-carboxylic acid and (1-methylcyclohexyl)methanamine analogously to Example 1.10 step 3.

LC-MS (Method A): Rt 2.43 min; MS m/z 362.2=[M+H]+

1H NMR (500 MHz, DMSO-d6) δ 12.46 (br s, 1H), 8.17 (t, J=6.2 Hz, 1H), 8.01 (br s, 1H), 7.70-7.61 (m, 1H), 7.57-7.39 (m, 1H), 7.36-7.28 (m, 4H), 7.27-7.20 (m, 1H), 4.20 (s, 2H), 3.17 (d, J=6.4 Hz, 2H), 1.55-1.46 (m, 2H), 1.46-1.18 (m, 8H), 0.88 (s, 3H).

### Example 1.10.2

### 2-Benzyl-N-(cyclooctylmethyl)-1H-benzimidazole-5-carboxamide

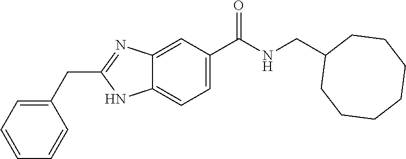 [SMILES: O=C(NCC1CCCCCCC1)c1ccc2[nH]c(Cc3ccccc3)nc2c1]

The title compound was prepared from 2-benzyl-1H-benzimidazole-5-carboxylic acid and cyclooctylmethanamine analogously to Example 1.10 step 3.

LC-MS (Method A): Rt 2.62 min; MS m/z 376.2=[M+H]+

1H NMR (500 MHz, DMSO-d6) δ 8.36 (t, J=5.7 Hz, 1H), 8.01 (br s, 1H), 7.67 (dd, J=8.4, 1.3 Hz, 1H), 7.49 (d, J=8.0 Hz, 1H), 7.35-7.31 (m, 4H), 7.28-7.20 (m, 1H), 4.20 (s, 2H), 3.10 (t, J=6.6 Hz, 2H), 1.86-1.78 (m, 1H), 1.70-1.60 (m, 4H), 1.57-1.38 (m, 8H), 1.30-1.22 (m, 2H).

### Example 2.1

### tert-Butyl N-[[1-[2-[(2-benzyl-1H-benzimidazol-5-yl)amino]-2-oxo-ethyl]cyclohexyl]methyl]carbamate

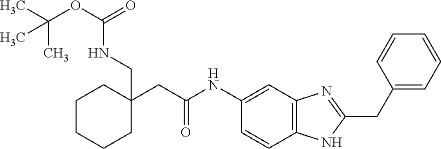 [SMILES: CC(C)(C)OC(=O)NCC1(CC(=O)Nc2ccc3[nH]c(Cc4ccccc4)nc3c2)CCCCC1]

2-Benzyl-1H-benzimidazol-5-amine (Intermediate A)(50 mg, 0.22 mmol) was added to a solution of 241-Rtert-butoxycarbonylamino)methylicyclohexyllacetic acid (67 mg, 0.25 mmol), HATU (102 mg, 0.27 mmol) and DIPEA (0.12 mL, 0.67 mmol) in DMF (2 mL) and the reaction mixture was stirred for 18 h. The resulting mixture was partitioned between EtOAc (10 mL) and water (10 mL) and the phases were separated. The organic phase was washed with 1M aq. LiOH (10 mL) and brine (10 mL). The combined organic extracts were dried over MgSO₄ and concentrated in vacuo to afford a yellow oil. The oil was purified by preparative HPLC (basic pH, early elution method) to afford the title compound as a colourless solid.

LC-MS (Method A): Rt 2.61 min; MS m/z 477.3=[M+H]+

1H NMR (500 MHz,Methanol-d4) δ 7.95 (s, 1H), 7.59-7.15 (m, 7H), 4.23 (s, 2H), 3.21 (s, 2H), 2.33 (s, 2H), 1.67-1.55 (m, 4H), 1.53-1.44 (m, 13H), 1.44-1.31 (m, 2H).

### Example 2.1.1

### N-(2-Benzyl-1H-benzimidazol-5-yl)-2-(4,4-difluorocyclohexypacetamide

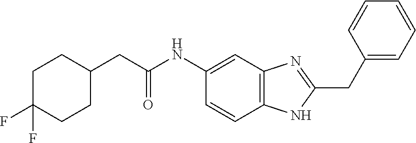 [SMILES: O=C(CC1CCC(F)(F)CC1)Nc1ccc2[nH]c(Cc3ccccc3)nc2c1]

The title compound was prepared from 2-benzyl-1H-benzimidazol-5-amine (Intermediate A) and 2-(4,4-difluorocyclohexyl)acetic acid analogously to Example 2.1.

LC-MS (Method A): Rt 1.95 min; MS m/z 384.2=[M+H]+

1H NMR (500 MHz, Methanol-d4) δ 7.92 (s, 1H), 7.43 (br. s, 1H), 7.34-7.28 (m, 4H), 7.28-7.11 (m, 2H), 4.20 (s, 2H), 2.32 (d, J=7.2 Hz, 2H), 2.10-1.92 (m, 3H), 1.91-1.70 (m, 4H), 1.44-1.31 (m, 2H).

### Example 2.2

### tert-Butyl N-[1-[5-[[2-(2-adamantyl)acetyl]amino]-1H-benzimidazol-2-yl]-2-methoxy-ethyl]carbamate

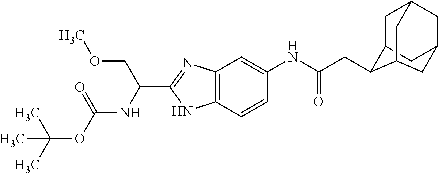 [SMILES: COCC(NC(=O)OC(C)(C)C)c1nc2cc(NC(=O)CC3C4CC5CC(C4)CC3C5)ccc2[nH]1]

To a stirred solution of 2-(tert-butoxycarbonylamino)-3-methoxy-propanoic acid (36 mg, 0.16 mmol), 2-(2-adamantyl)-N-(3,4-diaminophenyl)acetamide (Intermediate B)(95%, 50 mg, 0.16 mmol) and DIPEA (36 μL, 0.21 mmol) in DMF (1 mL) was added HATU (60 mg, 0.16 mmol) and the reaction mixture was stirred at room temperature for 2 h. The resulting mixture was concentrated in vacuo and the residue was diluted with sat. NaHCO₃ (10 mL) and EtOAc (10 mL). The organic layer was separated, washed with water (2 x 5 mL), brine (2×5 mL), dried over Na₂SO₄ and concentrated in vacuo. The crude material was dissolved in acetic acid (1 mL) and stirred at 70° C. for 2 h. The resulting mixture was concentrated in vacuo and the residue was partitioned between sat. NaHCO₃ (10 mL) and EtOAc (10 mL). The organic layer was separated, washed with water (2×5 mL) and dried over Na₂SO₄. The crude material was purified by preparative HPLC (acidic pH, early elution method) to afford the title compound as an off-white powder.

LC-MS (Method A): Rt 2.80 min; MS m/z 483.3=[M+H]+

1H NMR (500 MHz, Methanol-d4) δ 7.98 (d, J=1.4 Hz, 1H), 7.47 (d, J=8.7 Hz, 1H), 7.25 (dd, J=8.7, 1.8 Hz, 1H), 5.16-4.97 (m, 1H), 3.85-3.65 (m, 2H), 3.36 (s, 3H), 2.56 (d, J=7.7 Hz, 2H), 2.36 (t, J=7.6 Hz, 1H), 2.09-2.01 (m, 2H), 1.96-1.82 (m, 6H), 1.82-1.74 (m, 4H), 1.68-1.58 (m, 2H), 1.52-1.24 (m, 9H).

The compounds of the following tabulated Examples (Table Ex2.2) were prepared analogously to Example 2.2 from 2-(2-adamantyl)-N-(3,4-diaminophenyl)acetamide (Intermediate B) and the appropriate commercially available acid.

| Ex. | Structure and Name | Retention Time, [M + H]+, 1H NMR |
|---|---|---|
| 2.2.1 | 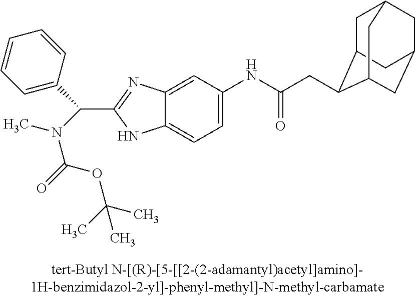 [SMILES: CN(C(=O)OC(C)(C)C)[C@H](c1ccccc1)c1nc2cc(NC(=O)CC3C4CC5CC(C4)CC3C5)ccc2[nH]1] | LC-MS (Method A): Rt 3.52 min; MS m/z 529.4 = [M + H]+ 1H NMR (500 MHz, DMSO-d6) δ 12.35 (s, 1H), 9.98 − 9.74 (m, 1H), 8.10 − 7.87 (m, 1H), 7.56 − 7.09 (m, 7H), 6.63 (br. s, 1H), 2.76 (s, 3H), 2.46 (d, J = 7.5 Hz, 2H), 2.28 − 2.20 (m, 1H), 2.00 − 1.91 (m, 2H), 1.89 − 1.65 (m, 10H), 1.57 − 1.49 (m, 2H), 1.40 (s, 9H). |
| 2.2.2 | 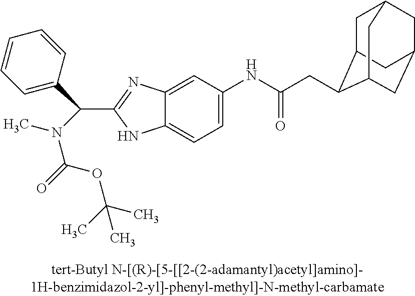 [SMILES: CN(C(=O)OC(C)(C)C)[C@@H](c1ccccc1)c1nc2cc(NC(=O)CC3C4CC5CC(C4)CC3C5)ccc2[nH]1] | LC-MS (Method A): Rt 3.51 min; MS m/z 529.4 = [M + H]+ 1H NMR (500 MHz, DMSO-d6) δ 12.47 − 12.25 (m, 1H), 9.98 − 9.73 (m, 1H), 8.09 − 7.89 (m, 1H), 7.59 − 7.06 (m, 7H), 6.62 (br. s, 1H), 2.76 (s, 3H), 2.46 (d, J = 7.7 Hz, 2H), 2.28 − 2.21 (m, 1H), 1.99 − 1.91 (m, 2H), 1.88 − 1.66 (m, 10H), 1.57 − 1.48 (m, 2H), 1.40 (s, 9H). |
| 2.2.3 | 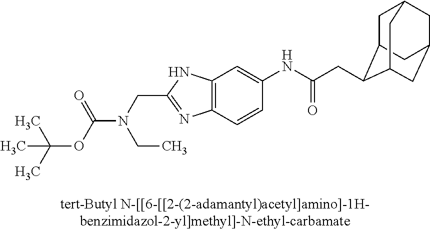 [SMILES: CCN(Cc1nc2ccc(NC(=O)CC3C4CC5CC(C4)CC3C5)cc2[nH]1)C(=O)OC(C)(C)C] | LC-MS (Method A): Rt 3.02 min; MS m/z 467.5 = [M + H]+ 1H NMR (250 MHz, DMSO-d6 VT NMR @353K) δ 11.81 (s, 1H), 9.54 (s, 1H), 8.01 − 7.74 (m, 1H), 7.50 − 7.11 (m, 2H), 4.54 (s, 2H), 3.33 (q, J = 7.1 Hz, 2H), 2.48 − 2.44 (obscured m, 2H), 2.32 − 2.23 (m, 1H), 2.03 − 1.70 (m, 12H), 1.55 (d, J = 12.4 Hz, 2H), 1.40 (s, 9H), 1.07 (t, J = 7.1 Hz, 3H). |
| 2.2.4 | 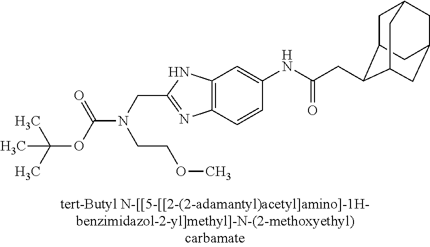 [SMILES: COCCN(Cc1nc2ccc(NC(=O)CC3C4CC5CC(C4)CC3C5)cc2[nH]1)C(=O)OC(C)(C)C] | LC-MS (Method A): Rt 2.77 min; MS m/z 497.3 = [M + H]+ 1H NMR (500 MHz, DMSO-d6 + 1 drop D₂O) δ 7.93 (br s, 1H), 7.40 (d, J = 7.8 Hz, 1H), 7.17 (br s, 1H), 4.54 (s, 2H), 3.52 − 3.36 (m, 4H), 3.21 (s, 3H), 2.43 (d, J = 7.7 Hz, 2H), 2.20 (t, J = 7.4 Hz, 1H), 1.91 (d, J = 12.5 Hz, 2H), 1.85 − 1.74 (m, 4H), 1.74 − 1.61 (m, 6H), 1.49 (d, J = 12.5 Hz, 2H), 1.44 − 1.12 (m, 9H). |
| 2.2.5 | 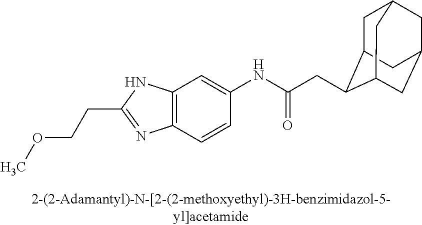 [SMILES: COCCc1nc2ccc(NC(=O)CC3C4CC5CC(C4)CC3C5)cc2[nH]1] | LC-MS (Method A): Rt 2.36 min; MS m/z 368.3 = [M + H]+ 1H NMR (500 MHz, DMSO-d6) δ 12.02 (s, 1H), 9.81 (s, 1H), 7.95 (s, 1H), 7.43 − 7.02 (m, 2H), 3.73 (t, J = 6.6 Hz, 2H), 3.26 (s, 3H), 3.00 (t, J = 6.6 Hz, 2H), 2.45 (d, J = 7.6 Hz, 2H), 2.24 (t, J = 7.6 Hz, 1H), 1.98 − 1.92 (m, 2H), 1.87 − 1.78 (m, 4H), 1.78 − 1.65 (m, 6H), 1.55 − 1.48 (m, 2H). LC-MS (Method A): Rt 2.24 min; MS m/z 382.3 = [M + H]+ |
| 2.2.6 | 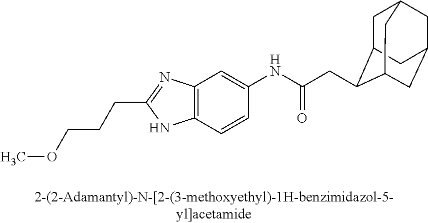 [SMILES: COCCCc1nc2cc(NC(=O)CC3C4CC5CC(C4)CC3C5)ccc2[nH]1] | 1H NMR (500 MHz, DMSO-d6) δ 12.01 (s, 1H), 9.80 (s, 1H), 7.93 (s, 1H), 7.44-7.02 (m, 2H), 3.38 (t, J = 6.4 Hz, 2H), 3.24 (s, 3H), 2.80 (t, J = 7.6 Hz, 2H), 2.45 (d, J = 7.6 Hz, 2H), 2.24 (t, J = 7.5 Hz, 1H), 2.01-1.92 (m, 4H), 1.89-1.84 (m, 1H), 1.81 (d, J = 11.2 Hz, 3H), 1.78-1.66 (m, 6H), 1.52 (d, J = 12.4 Hz, 2H). |

### Example 2.3

### tert-Butyl N-[[5-[[2-(2-adamanty)acetyl]amino]-1H-benzimidazol-2-yl]methyl]-N-methyl-carbamate

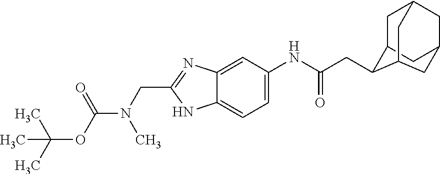 [SMILES: CN(Cc1nc2cc(NC(=O)CC3C4CC5CC(C4)CC3C5)ccc2[nH]1)C(=O)OC(C)(C)C]

Step 1: tert-Butyl N-methyl-N-[(5-nitro-1H-benzimidazol-2-yl)methyl]carbamate

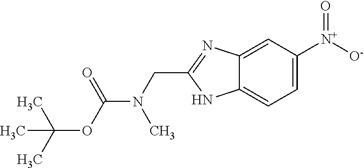 [SMILES: CN(Cc1nc2cc([N+](=O)[O-])ccc2[nH]1)C(=O)OC(C)(C)C]

A solution of 2-[tert-butoxycarbonyl(methyl)amino]acetic acid (1.4 g, 7.4 mmol) and HATU (3.38 g, 8.88 mmol) in DMF (15 mL) was treated with 4-nitrobenzene-1,2-diamine (1.36 g, 8.88 mmol) and DIPEA (2.58 mL, 14.8 mmol) and the mixture was stirred at room temperature for 16 h. The resulting mixture was diluted with EtOAc (25 mL) and washed with water (3×25 mL). The combined aqueous portions were back-extracted with EtOAc (25 mL) and the combined organic extracts were washed with saturated aqueous NaHCO₃ (25 mL), brine (25 mL), dried over MgSO₄ and concentrated in vacuo. The residue was dissolved in acetic acid (10 mL) and stirred at 70° C. for 3 h. The resulting mixture was concentrated in vacuo and the residue was partitioned between NaHCO₃ (50 mL) and EtOAc (50 mL). The organic layer was separated, washed with water (3×50 mL), brine (50 mL), dried over MgSO4, filtered and concentrated in vacuo. Purification by chromatography on silica eluting with 0-100% EtOAc in heptanes afforded the title compound as an orange solid.

LC-MS (Method A): Rt 2.67 min; MS m/z 307=[M+H]+

1H NMR (500 MHz, DMSO-d6) δ 12.97 (br. s, 1H), 8.43 (s, 1H), 8.08 (dd, J=8.9, 2.3 Hz, 1H), 7.70 (d, J=8.4 Hz, 1H), 4.66 (s, 2H), 2.96 (s, 3H), 1.58-1.19 (m, 9H).

Step 2: tert-Butyl N-[(5-amino-1H-benzimidazol-2-yl)methyl]-N-methyl-carbamate

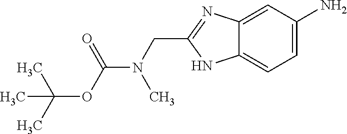 [SMILES: CN(Cc1nc2cc(N)ccc2[nH]1)C(=O)OC(C)(C)C]

A solution of tert-butyl N-methyl-N-[(5-nitro-1H-benzimidazol-2-yl)methyl]carbamate (step 1) (99%, 500 mg, 1.62 mmol) in EtOH (10 mL) was purged with nitrogen (3 times) and treated with 10% Pd/C (50% wet) (5%, 86 mg, 0.04 mmol) The mixture was placed under a hydrogen atmosphere and stirred at room temperature for 16 h. The resulting mixture was filtered through Celite® (filter material) and concentrated in vacuo to afford the title compound.

LC-MS (Method E): Rt 0.71 min; MS m/z 277.1=[M+H]+

1H NMR (500 MHz, DMSO-d6) δ 11.84-11.53 (m, 1H), 7.27-7.05 (m, 1H), 6.75-6.40 (m, 2H), 4.91 -4.60 (m, 2H), 4.46 (s, 2H), 2.86 (s, 3H), 1.53-1.25 (m, 9H).

Step 3: tert-Butyl N-[[5-[[2-(2-adamantypacetyl]amino]-1H-benzimidazol-2-yllmethyll-N-methyl-carbamate

A solution of 2-(2-adamantyl)acetic acid (Intermediate B step 3) (103 mg, 0.53 mmol) and HATU (243 mg, 0.64 mmol) in DMF (3 mL) was treated with DIPEA (0.19 mL, 1.06 mmol) and teat-butyl N-[(5-amino-1H-benzimidazol-2-yl)methyl]-N-methyl-carbamate (step 2) (98%, 150 mg, 0.53 mmol) and the mixture was stirred at room temperature for 16 h. The resulting mixture was diluted with EtOAc (20 mL) and washed with water (3×20 mL). The organic portion was dried over MgSO₄ and concentrated in vacuo. Purification by C18 reverse phase chromatography eluting with 10-100% MeCN in water (0.1% formic acid) afforded the title compound as an off-white solid.

LC-MS (Method A): Rt 2.86 min; MS m/z 453.4=[M+H]+

1H NMR (500 MHz, Methanol-d4) δ 7.98 (s, 1H), 7.47 (d, J=8.8 Hz, 1H), 7.30-7.22 (m, 1H), 4.65 (s, 2H), 3.06-2.94 (m, 3H), 2.57 (d, J=7.7 Hz, 2H), 2.40-2.33 (m, 1H), 2.09-2.01 (m, 2H), 1.96-1.83 (m, 6H), 1.82-1.75 (m, 4H), 1.64 (d, J=12.6 Hz, 2H), 1.57-1.29 (m, 9H).

### Example 2.4

### N-(Cycloheptylmethyl)-2-(2,3-dihydrobenzofuran-3-yl)-1H-benzimidazole-5-carboxamide

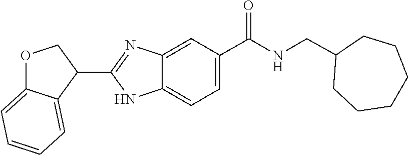 [SMILES: O=C(NCC1CCCCCC1)c1ccc2[nH]c(C3COc4ccccc43)nc2c1]

To a solution of 2-(2,3-dihydrobenzofuran-3-yl)-1H-benzimidazole-5-carboxylic acid (Intermediate D) (90%, 61 mg, 0.20 mmol) in DMF (1 mL) was added DIPEA (38 μL, 0.22 mmol) and HATU (82 mg, 0.22 mmol) followed by a solution of cycloheptylmethanamine (27 mg, 0.22 mmol) in DMF (1 mL) and the mixture stirred at room temperature for 1 h. The resulting mixture was diluted with water and extracted with EtOAc (5 mL). The organic extract was washed with brine (5 mL), dried over Na₂SO₄ and concentrated in vacuo. The crude residue was purified by preparative HPLC (basic pH, early elution method) to afford the title compound as a colourless powder.

LC-MS (Method A): Rt 2.91 min; MS m/z 390.2=[M+H]+

1H NMR (500 MHz, DMSO-d6) δ 8.38 (t, J=5.2 Hz, 1H), 8.04 (br s, 1H), 7.69 (d, J=8.5 Hz, 1H), 7.52 (d, J=8.2 Hz, 1H), 7.29 (d, J=7.4 Hz, 1H), 7.18 (t, J=7.7 Hz, 1H), 6.90-6.84 (m, 2H), 5.08 (dd, J=9.6, 6.9 Hz, 1H), 5.05-5.00 (m, 1H), 4.98-4.93 (m, 1H), 3.11 (t, J=6.3 Hz, 2H), 1.80-1.70 (m, 3H), 1.66-1.61 (m, 2H), 1.58-1.52 (m, 2H), 1.51-1.43 (m, 2H), 1.42-1.35 (m, 2H), 1.21-1.14 (m, 2H).

### Example 2.5

### 2-(2-Adamantyl)-N-12-[hydroxy(phenyl)methyl]-1H-benzimidazol-5-yljacetamide

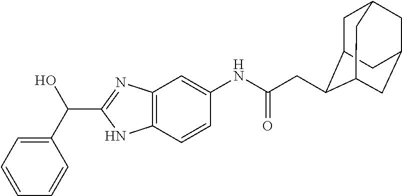 [SMILES: O=C(CC1C2CC3CC(C2)CC1C3)Nc1ccc2[nH]c(C(O)c3ccccc3)nc2c1]

Step 1: 2-(2-Adamantyl)-N- [2-[[methoxy(pheny)methyl]-1H-benzimidazol-5-yl]acetamide

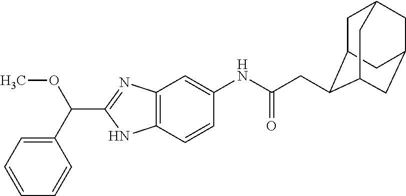 [SMILES: COC(c1ccccc1)c1nc2cc(NC(=O)CC3C4CC5CC(C4)CC3C5)ccc2[nH]1]

The title compound was prepared from 2-(2-adamantyl)-N-(3,4-diaminophenyl)acetamide (Intermediate B) and 2-methoxy-2-phenyl-acetic acid analogously to Example 2.2.

LC-MS (Method A): Rt 2.88 min; MS m/z 430.3=[M+H]+

1H NMR (500 MHz, DMSO-d6) δ 12.42-12.32 (m, 1H), 9.94-9.70 (m, 1H), 8.06-7.80 (m, 1H), 7.51 -7.06 (m, 7H), 5.61-5.49 (m, 1H), 3.36 (s, 3H), 2.48-2.41 (m, 2H), 2.27-2.19 (m, 1H), 1.98-1.91 (m, 2H), 1.88-1.64 (m, 10H), 1.56-1.47 (m, 2H).

Step 2: 2-(2-Adamantyl)-N424hydroxy(phenyl)methyll -1H-benzimidazol-5-yl] acetamide

1M BBr₃ in DCM (0.37 mL, 0.37 mmol) was added dropwise to solution of 2-(2-adamantyl)-N[2-[methoxy(phenyl)methyl]-1H-benzimidazol-5-yl]acetamide (step 1) (53 mg, 0.12 mmol) in DCM (3 mL) and the mixture was stirred at room temperature overnight. The resulting mixture was allowed to stand at room temperature for 2 days whereupon the solvent evaporated to afford a white/yellow solid. The solid was suspended in water (5 mL) and sonicated. The acidic aqueous was adjusted to pH 8 using saturated aqueous sodium bicarbonate solution. EtOAc (10 mL) was added and the mixture was sonicated until all solids dissolved. The organic layer was separated, washed with water and passed through a phase separating column. The mixture was concentrated in vacuo and the crude product was suspended in MeOH (1 mL) and filtered. The filtrate was concentrated in vacuo then the residue was suspended MeOH (1 mL) and briefly heated and sonicated. After cooling to room temperature, the suspension was filtered, the solids were discarded and the filtrate purified by C18 reverse phase chromatography eluting with 10-100% MeCN in water (+0.1% formic acid) to afford the title compound as a colourless solid.

LC-MS (Method A): Rt 2.53 min; MS m/z 416.3=[M+H]+

1H NMR (500 MHz, Methanol-d4) δ 7.93 (d, J=1.8 Hz, 1H), 7.53-7.48 (m, 2H), 7.45 (d, J=8.6 Hz, 1H), 7.38-7.31 (m, 2H), 7.31-7.25 (m, 1H), 7.25-7.20 (m, 1H), 5.97 (s, 1H), 2.59-2.52 (m, 2H), 2.39 -2.32 (m, 1H), 2.09-2.00 (m, 2H), 1.95-1.73 (m, 10H), 1.68-1.58 (m, 2H).

### Example 3.1

### 2-Cyclohexyl-N-(2-phenyl-1H-benzimidazol-5-yl)acetamide

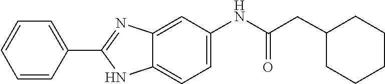 [SMILES: O=C(CC1CCCCC1)Nc1ccc2[nH]c(-c3ccccc3)nc2c1]

To a cooled (0° C.) solution of 2-phenyl-1H-benzimidazol-5-amine (50 mg, 0.24 mmol) and DIPEA (84 μL, 0.48 mmol) in DCM (5 mL) was added dropwise 2-cyclohexylacetyl chloride (42 mg, 0.26 mmol) and the mixture was stirred at room temperature for 1 h. The resulting mixture was washed with a saturated solution of sodium bicarbonate (5 mL), dried over Na₂SO₄ and concentrated in vacuo. Purification by preparative HPLC (basic pH, early elution method) afforded the title compound.

LC-MS (Method A): Rt 2.29 min; MS m/z 334.2=[M+H]+

1H NMR (500 MHz, DMSO-d6) 6=12.96-12.55 (m, 1H), 9.98-9.71 (m, 1H), 8.27-7.95 (m, 3H), 7.66 -7.06 (m, 5H), 2.29-2.15 (m, 2H), 1.88-1.54 (m, 6H), 1.38-1.09 (m, 3H), 1.06-0.90 (m, 2H).

### Example 3.2

### N-(2-Benzyl-1H-benzimidazol-5-yl)adamantane-1-carboxamide

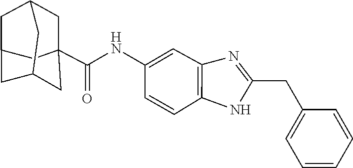 [SMILES: O=C(Nc1ccc2[nH]c(Cc3ccccc3)nc2c1)C12CC3CC(CC(C3)C1)C2]

To a cooled (0° C.) solution of 2-benzyl-1H-benzimidazol-5-amine (Intermediate A) (60 mg, 0.27 mmol) and DIPEA (56 μL, 0.32 mmol) in DMF (1 mL) was added adamantane-l-carbonyl chloride (59 mg, 0.30 mmol). The solution was warmed to room temperature and stirred for 1 h. The resulting mixture was diluted with EtOAc (5 mL) and washed with water (5 mL), brine (5 mL), dried over Na₂SO₄ and concentrated in vacuo. The residue was suspended in water (1.5 mL) and MeCN (0.5 mL) and filtered, washing with ether and heptanes then dried under a flow of nitrogen to afford the title compound.

LC-MS (Method A): Rt 2.50 min; MS m/z 386.3=[M+H]+

1H NMR (500 MHz, DMSO-d6) δ 12.14 (s, 1H), 9.06-8.97 (m, 1H), 7.92-7.82 (m, 1H), 7.41-7.21 (m, 7H), 4.13 (s, 2H), 2.04-1.99 (m, 3H), 1.93-1.90 (m, 6H), 1.72-1.69 (m, 6H).

### Example 3.3

### N-(Cycloheptylmethyl)-7-fluoro-2-[(3-hydroxyphenyl)methyl]-1H-benzimidazole-5-carboxamide

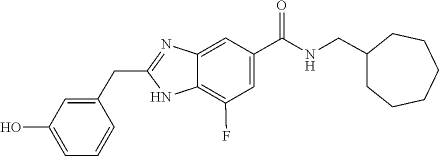 [SMILES: O=C(NCC1CCCCCC1)c1cc(F)c2[nH]c(Cc3cccc(O)c3)nc2c1]

Step 1: 2[(3-Benzyloxyphenyl)methyl1-5-bromo-7-fluoro-1H-benzimidazole

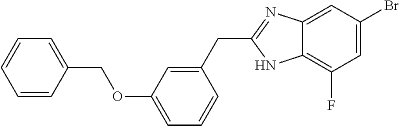 [SMILES: Fc1cc(Br)cc2nc(Cc3cccc(OCc4ccccc4)c3)[nH]c12]

To a solution of 2-(3-benzyloxyphenyl)acetic acid (650 mg, 2.68 mmol) in DMF (10 mL) was added HATU (1113 mg, 2.93 mmol) followed by DIPEA (0.85 mL, 4.88 mmol). The mixture was stirred for 30 mins at room temperature under nitrogen and then 5-bromo-3-fluoro-benzene-1,2-diamine (500 mg, 2.44 mmol) was added. After stirring at room temperature overnight, the resulting mixture was diluted with EtOAc (50 mL) and washed with water (3×50 mL) and brine (3×50 mL). The organic portion was dried over Na₂SO₄ and concentrated in vacuo. The resulting black oil was taken up in acetic acid (10 mL) and heated at 70° C. for 3 h and then allowed to cool to room temperature. The mixture was diluted with water (100 mL) and then extracted with EtOAc (50 mL). The organic extract was washed with water (2×50 mL), sat. aq. NaHCO₃ (50 mL), brine (50 mL), dried over Na₂SO₄ and concentrated in vacuo. The crude product was purified by chromatography on silica eluting with 0-100% EtOAc in heptanes to afford the title compound as a light brown solid.

LC-MS (Method E): Rt 1.31 min; MS m/z 411.0/413.0=[M+H]+

1H NMR (250 MHz, DMSO-d6) δ 12.71 (br. s, 1H), 7.51 (s, 1H), 7.45-7.29 (m, 5H), 7.28-7.19 (m, 2H), 7.01-6.97 (m, 1H), 6.93-6.86 (m, 2H), 5.07 (s, 2H), 4.15 (s, 2H).

Step 2: 24(3-B enzyloxyphenyl)methyll -N-(cycloheptylmethyl)-7-fluoro-1H-benzimidazole-5-carboxamide

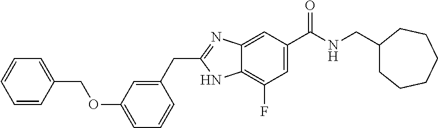 [SMILES: O=C(NCC1CCCCCC1)c1cc(F)c2[nH]c(Cc3cccc(OCc4ccccc4)c3)nc2c1]

All reagents charged to COware equipment (carbon monoxide generating system) according to the following procedure;

Chamber A was charged 2[(3-benzyloxyphenyl)methyl1-5-bromo-7-fluoro-1H-benzimidazole (step 1) (83%, 200 mg, 0.40 mmol), sodium carbonate (128 mg, 1.21 mmol) and XantPhos Pd-G3 (third generation G3 Buchwald precatalyst) (19 mg, 0.020 mmol). Toluene (5 mL) was added followed by cycloheptylmethanamine (77 mg, 0.61 mmol). The mixture was de-gassed with nitrogen for 5 mins. After this time formic acid (46 μL, 1.21 mmol) in toluene (5 mL) was added to chamber B followed by mesyl chloride (94 μL, 1.21 mmol). The apparatus was de-gassed with nitrogen for a further 2 mins and then sealed. TEA (338 μL, 2.42 mmol) was added to chamber B (to generate CO gas). The sealed system was heated at 100° C. overnight. The resulting mixture from chamber A was concentrated in vacuo and the residue was taken up in EtOAc (50 mL). The mixture was washed with water (2×25 mL), brine (25 mL), dried over Na₂SO₄ and concentrated in vacuo. The crude material was purified by chromatography on silica eluting with 0-100% EtOAc in heptanes to afford the title compound as a light yellow solid.

LC-MS (Method E): Rt 1.35 min; MS m/z 486.2=[M+H]+

1H NMR (500 MHz, DMSO-d6) δ 13.20-12.70 (m, 1H), 8.46 (t, J=5.6 Hz, 1H), 8.00-7.74 (m, 1H), 7.48 (d, J=11.5 Hz, 1H), 7.43 (d, J=7.1 Hz, 2H), 7.36 (t, J=7.3 Hz, 2H), 7.33-7.29 (m, 1H), 7.25 (t, J=7.9 Hz, 1H), 7.01 (s, 1H), 6.94-6.88 (m, 2H), 5.08 (s, 2H), 4.19 (s, 2H), 3.11 (t, J=6.3 Hz, 2H), 1.80-1.67 (m, 3H), 1.67-1.59 (m, 2H), 1.58-1.34 (m, 6H), 1.22-1.13 (m, 2H). Step 3: N-(Cycloheptylmethyl)-7-fluoro-24(3-hydroxyphenyl)methyll -1H-benzimidazole -5-carboxamide To a solution of 24(3-benzyloxyphenyl)methyll-N-(cycloheptylmethyl)-7-fluoro-1H-benzimidazole-5-carboxamide (step 2) (75 mg, 0.15 mmol) in EtOH (25 mL) was added 10% Pd/C (50% wet) (5%, 33 mg, 0.015 mmol) and the mixture placed under a hydrogen atmosphere and stirred at room temperature for 16 h. The resulting mixture was filtered through Celite® (filter material), washing through with EtOH (20 mL). The filtrate was concentrated in vacuo and the crude product was by preparative HPLC (basic pH, early elution method) afforded the title compound as a white solid.

LC-MS (Method A): Rt 2.96 min; MS m/z 396.2=[M+H]+

1H NMR (500 MHz, DMSO-d6) δ 12.69 (br. s, 1H), 9.35 (s, 1H), 8.45 (t, J=5.7 Hz, 1H), 7.83 (br. s, 1H), 7.47 (d, J=11.8 Hz, 1H), 7.11 (t, J=7.8 Hz, 1H), 6.74 (d, J=7.7 Hz, 1H), 6.71-6.69 (m, 1H), 6.63 (dd, J=8.0, 1.9 Hz, 1H), 4.12 (s, 2H), 3.10 (t, J=6.3 Hz, 2H), 1.80-1.67 (m, 3H), 1.67-1.58 (m, 2H), 1.58-1.33 (m, 6H), 1.21-1.12 (m, 2H).

### Example 3.3.1

### N-(Cycloheptylmethyl)-6-fluoro-2-[(3-hydroxyphenyl)methyl]-1H-benzimidazole-5-carboxamide

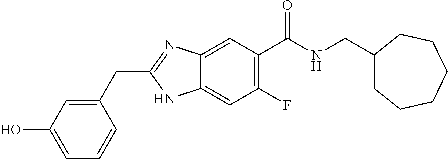 [SMILES: O=C(NCC1CCCCCC1)c1cc2nc(Cc3cccc(O)c3)[nH]c2cc1F]

The title compound was prepared analogously to Example 3.3 (steps 1-3) by replacing 5-bromo-3-fluoro-benzene-1,2-diamine (step 1) with 4-bromo-5-fluoro-benzene-1,2-diamine.

LC-MS (Method A): Rt 2.71 min; MS m/z 396.2=[M+H]+

1H NMR (500 MHz, DMSO-d6) δ 9.44 (br s, 1H), 8.28-8.23 (m, 1H), 7.75 (d, J=6.2 Hz, 1H), 7.48 (d, J=10.5 Hz, 1H), 7.13 (t, J=7.8 Hz, 1H), 6.75 (d, J=7.7 Hz, 1H), 6.73-6.71 (m, 1H), 6.67 (dd, J=8.0, 1.8 Hz, 1H), 4.21 (s, 2H), 3.10 (t, J=6.2 Hz, 2H), 1.75-1.70 (m, 3H), 1.67-1.61 (m, 2H), 1.57-1.52 (m, 2H), 1.50-1.45 (m, 2H), 1.42-1.36 (m, 2H), 1.22-1.15 (m, 2H).

### Example 3.3.2

### N-(Cycloheptylmethyl)-4-fluoro-2-[(3-hydroxyphenyl)methyl]-1H-benzimidazole-5-carboxamide

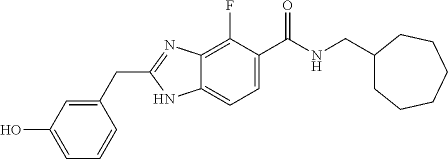 [SMILES: O=C(NCC1CCCCCC1)c1ccc2[nH]c(Cc3cccc(O)c3)nc2c1F]

The title compound was prepared analogously to Example 3.3 (steps 1-3) by replacing 5-bromo-3-fluoro-benzene-1,2-diamine (step 1) with 4-bromo-3-fluoro-benzene-1,2-diamine.

LC-MS (Method A): Rt 2.90 min; MS m/z 396.2=[M+H]+

1H NMR (500 MHz, DMSO-d6) δ 9.35 (br s, 1H), 8.14 (br s, 1H), 7.37-7.33 (m, 1H), 7.31-7.26 (m, 1H), 7.10 (t, J=7.8 Hz, 1H), 6.73 (d, J=7.7 Hz, 1H), 6.71-6.67 (m, 1H), 6.62 (dd, J=8.0, 1.9 Hz, 1H), 4.10 (s, 2H), 3.11 (t, J=6.2 Hz, 2H), 1.77-1.70 (m, 3H), 1.67-1.60 (m, 2H), 1.58-1.52 (m, 2H), 1.51 -1.44 (m, 2H), 1.43-1.35 (m, 2H), 1.22-1.14 (m, 2H).

### Example 3.4

### tert-Butyl N-[2-[5-[[2-(2-adamantyl)acetyl[amino]-1H-benzimidazol-2-yl]ethyl]carbamate

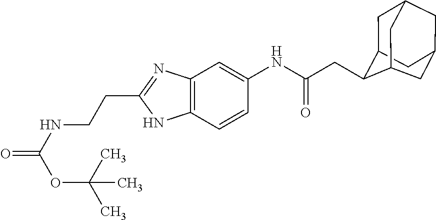 [SMILES: CC(C)(C)OC(=O)NCCc1nc2cc(NC(=O)CC3C4CC5CC(C4)CC3C5)ccc2[nH]1]

To a stirred solution of 3-(tert-butoxycarbonylamino)propanoic acid (75 mg, 0.40 mmol), 2-(2-adamantyl)-N-(3,4-diaminophenyl)acetamide (Intermediate B) (95%, 125 mg, 0.40 mmol) and DIPEA (90 μL, 0.52 mmol) in DMF (2 mL) was added HATU (151 mg, 0.40 mmol) and the reaction mixture was stirred at room temperature for 2 h. The resulting mixture was concentrated in vacuo and the residue was partitioned between sat. NaHCO₃ (10 mL) and EtOAc (10 mL). The organic layer was separated, washed with water (2×5 mL), brine (2×5 mL), dried over Na₂SO₄ and concentrated in vacuo. The resulting crude material was dissolved in acetic acid (2 mL) and stirred at 70° C. for 2 h. The mixture was concentrated in vacuo and the residue was partitioned between sat. NaHCO₃ (10 mL) and EtOAc (10 mL). The organic layer was washed with water (2×5 mL) and dried over Na₂SO₄ The crude material was purified by preparative HPLC (basic pH, early elution method) to afford the title compound as an off-white powder.

LC-MS (Method A): Rt 2.46 min; MS m/z 453.3=[M+H]+

1H NMR (500 MHz, Methanol-d4) δ 7.92 (s, 1H), 7.43 (br s, 1H), 7.18 (br s, 1H), 3.50 (t, J=7.0 Hz, 2H), 3.02 (t, J=6.9 Hz, 2H), 2.56 (d, J=7.7 Hz, 2H), 2.36 (t, J=7.7 Hz, 1H), 2.05 (d, J=14.6 Hz, 2H), 1.98-1.83 (m, 6H), 1.83-1.73 (m, 4H), 1.64 (d, J=12.5 Hz, 2H), 1.45-1.20 (m, 9H).

### Example 3.4.1

### 2-(2-Adamantyl)-N-12-1(3,5-dimethylisoxazol-4-yOmethyll-1H-benzimidazol-5-yljacetamide

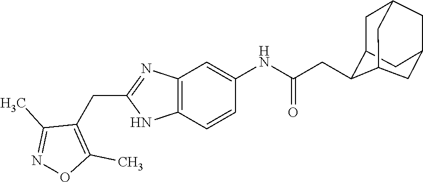 [SMILES: Cc1noc(C)c1Cc1nc2cc(NC(=O)CC3C4CC5CC(C4)CC3C5)ccc2[nH]1]

The title compound was prepared from 2-(2-adamantyl)-N-(3,4-diaminophenyl)acetamide (Intermediate B) and 2-(3,5-dimethylisoxazol-4-yl)acetic acid analogously to Example 3.4.

LC-MS (Method A): Rt 2.56 min; MS m/z 419.4=[M+H]+

1H NMR (500 MHz, DMSO-d6) δ 12.06 (br. s, 1H), 9.82 (br. s, 1H), 8.07-7.78 (m, 1H), 7.48-7.03 (m, 2H), 3.90 (s, 2H), 2.45 (d, J=7.7 Hz, 2H), 2.32 (s, 3H), 2.26-2.20 (m, 1H), 2.08 (s, 3H), 1.98-1.90 (m, 2H), 1.89-1.66 (m, 10H), 1.56-1.47 (m, 2H).

### Example 3.5

### 2-Benzyl-N-(2,2-dimethylpropyl)-1H-benzimidazole-5-carboxamide

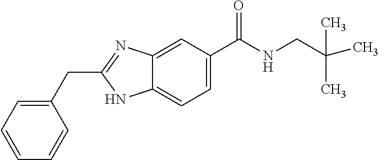 [SMILES: CC(C)(C)CNC(=O)c1ccc2[nH]c(Cc3ccccc3)nc2c1]

A solution of 2-benzyl-1H-benzimidazole-5-carboxylic acid (75 mg, 0.3 mmol) in DMF (2 mL) was treated with EDCI (63 mg, 0.33 mmol), DMAP (40 mg, 0.33 mmol) and HOAt (45 mg, 0.33 mmol). After stirring at room temperature for 5 mins, 2,2-dimethylpropan-l-amine (52 mg, 0.59 mmol)) was added and the reaction mixture was stirred under an inert atmosphere at room temperature for 16 h. The resulting mixture was diluted with EtOAc (20 mL) and washed with water (2×10 mL), brine (2×10 mL) and concentrated in vacuo. The crude product was purified by HPLC (acidic pH, standard elution method) to afford the title compound as a colourless solid.

LC-MS (Method A): Rt 1.89 min; MS m/z 322.2=[M+H]+

1H NMR (500 MHz, DMSO-d6) δ 12.69-12.33 (m, 1H), 8.29-8.20 (m, 1H), 8.16-7.86 (m, 1H), 7.67 (d, J=8.3 Hz, 1H), 7.59-7.39 (m, 1H), 7.36-7.28 (m, 4H), 7.27-7.20 (m, 1H), 4.20 (s, 2H), 3.12 (d, J =6.4 Hz, 2H), 0.90 (s, 9H).

### Example 3.5.1

### 2-Benzyl-N-(1,1,2,2-tetramethylpropyl)-1H-benzimidazole-5-carboxamide ET4146

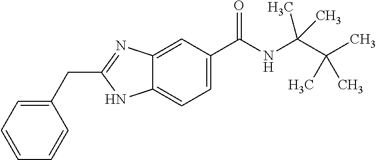 [SMILES: CC(C)(C)C(C)(C)NC(=O)c1ccc2[nH]c(Cc3ccccc3)nc2c1]

The title compound was prepared from 2-benzyl-1H-benzimidazole-5-carboxylic acid and 2,3,3-trimethylbutan-2-amine analogously to Example 3.5.

LC-MS (Method A): Rt 2.31 min; MS m/z 350.2=[M+H]+

1H NMR (500 MHz, DMSO-d6) δ 12.45 (s, 1H), 7.89 (s, 1H), 7.55 (dd, J=8.4, 1.4 Hz, 1H), 7.47 (d, J=8.3 Hz, 1H), 7.34-7.29 (m, 4H), 7.26-7.21 (m, 1H), 7.05 (s, 1H), 4.19 (s, 2H), 1.42 (s, 6H), 0.99 (s, 9H).

### Preparation of Intermediates

### Intermediate A

### 2-Benzyl-1H-benzimidazol-5-amine

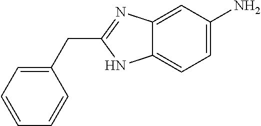 [SMILES: Nc1ccc2[nH]c(Cc3ccccc3)nc2c1]

Step 1: 2-Benzyl-6-nitro-1H-benzimidazole

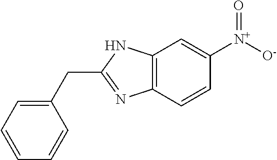 [SMILES: O=[N+]([O-])c1ccc2nc(Cc3ccccc3)[nH]c2c1]

A solution of DIPEA (4.56 mL, 26.12 mmol), 2-phenylacetic acid (1.60 g, 11.75 mmol) , HATU (4.47 g, 11.75 mmol) and 4-nitrobenzene-1,2-diamine (2.0 g, 13.06 mmol) in DMF (50 mL) was stirred at room temperature for 72 h. The resulting mixture was diluted with EtOAc (60 mL) and washed with water (2 x 50 mL), brine (2×50 mL), dried over MgSO₄ and concentrated in vacuo. The crude material was taken up in acetic acid (50 mL) and stirred at 60° C. for 20 h. The mixture was concentrated in vacuo and the resulting residue partitioned between EtOAc (30 mL) and cold sat. aq. NaHCO₃ solution (30 mL). The phases were separated and the organics were washed with water (2×30 mL), brine (30 mL), dried over MgSO₄ and concentrated in vacuo to afford the title compound as a red viscous oil.

LC-MS (Method E): Rt 1.00 min; MS m/z 254.0=[M+H]+

1H NMR (500 MHz, DMSO-d6) δ 12.98 (br s, 1H), 8.40 (s, 1H), 8.07 (dd, J=8.9, 2.3 Hz, 1H), 7.66 (d, J=8.9 Hz, 1H), 7.40-7.30 (m, 4H), 7.30-7.21 (m, 1H), 4.26 (s, 2H).

Step 2: 2-Benzyl-1H-benzimidazol -5-amine

To a cooled (0° C.) solution of 2-benzyl-5-nitro-1H-benzimidazole (step 1) (3.1 g, 12.24 mmol) in MeOH (40.5 mL) and acetic acid (13.5 mL) was added zinc powder (4.8 g, 73.44 mmol) and the reaction mixture was allowed to warm to room temperature and stirred for 20 mins. The resulting mixture was filtered through Celite® (filter material) washing through with MeOH. The filtrate was concentrated in vacuo and the crude residue dissolved in EtOAc (50 mL) and sat. aq. NaHCO₃ solution (50 mL). The resulting biphasic mixture was filtered then the phases of the filtrate separated. The aqueous layer was extracted with CHCl₃/IPA (2:1, 3×20 mL) and the combined organic portions were concentrated in vacuo. The crude material was dissolved in 3M aq. HC1 solution (heating required for dissolution) and then treated with 2M NaOH until a solid precipitate persisted. Filtration failed to isolate the product so the combined solid and filtrate was concentrated in vacuo to afford a brown oil. Purification by C18 reverse phase column chromatography eluting with 10-100% MeCN in water (0.1% formic acid) afforded the title compound as a pink glassy solid.

LC-MS (Method E): Rt 0.65 min; MS m/z 224.2=[M+H]+

1H NMR (500 MHz, DMSO-d6) δ 7.41-7.28 (m, 6H), 6.82 (d, J=1.7 Hz, 1H), 6.78 (dd, J=8.7, 2.0 Hz, 1H), 4.35 (s, 2H).

### Intermediate B

### 2-(2-Adamantyl)-N-(3,4-diaminophenyl)acetamide

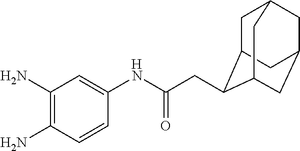

Step 1: Ethyl 2-(2-adamantylidene)acetate

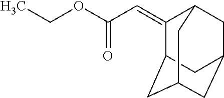 [SMILES: CCOC(=O)C=C1C2CC3CC(C2)CC1C3]

Ethyl 2-diethoxyphosphorylacetate (7.26 mL, 36.61 mmol) was added dropwise to a cooled (0° C.) suspension of NaH, 60% dispersion in mineral oil (1.86 g, 46.6 mmol) in THF (100 mL). After stirring at 0° C. for 30 mins, adamantan-2-one (5.0 g, 33.28 mmol) was added and the mixture was allowed to warm to room temperature and stirred for 2 hours. The resulting mixture was diluted with DCM (100 mL) and washed with water (100 mL). The aqueous portion was extracted with DCM (100 mL) and the combined organic extracts dried over MgSO₄ and concentrated in vacuo to afford a colourless oil. The oil was purified by chromatography on silica eluting with 0-20% EtOAc in heptanes to afford the title compound as a colourless oil.

LC-MS (Method B): Rt 1.43 min; MS m/z 221.3=[M+H]+

1H NMR (500 MHz, Chloroform-d) δ 5.58 (s, 1H), 4.14 (q, J=7.1 Hz, 2H), 4.06 (s, 1H), 2.43 (s, 1H), 2.00 -1.90 (m, 6H), 1.88-1.78 (m, 6H), 1.27 (t, J=7.1 Hz, 3H).

Step 2: Ethyl 2-(2-adamantyl)acetate

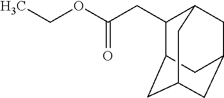 [SMILES: CCOC(=O)CC1C2CC3CC(C2)CC1C3]

A suspension of ethyl 2-(2-adamantylidene)acetate (step 1) (95%, 14.0 g, 60.37 mmol) and Pd/C (10%, 6.42 g, 6.04 mmol) in EtOH (125 mL) was placed under a hydrogen atmosphere and was stirred for 18 hours. The resulting mixture was filtered through glass filter paper and the filter cake washed with EtOH (2×10 mL). The filtrate was concentrated in vacuo to afford the title compound as a colourless oil.

LC-MS (Method B): Rt 1.47 min; MS m/z 223.0=[M+H]+

1H NMR (500 MHz, Chloroform-d) δ 4.12 (q, J=7.1 Hz, 2H), 2.44 (d, J=7.6 Hz, 2H), 2.23 (t, J=7.6 Hz, 1H), 1.91-1.75 (m, 8H), 1.71 (d, J=10.9 Hz, 4H), 1.62-1.50 (m, 3H), 1.25 (t, J=7.1 Hz, 3H). Step 3: 2-(2-Adamantyl)acetic acid

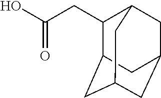 [SMILES: O=C(O)CC1C2CC3CC(C2)CC1C3]

A solution of ethyl 2-(2-adamantyl)acetate (step 2) (100%, 18.3 g, 82.31 mmol) in MeOH (200 mL) and 2M aq. sodium hydroxide (82.31 mL, 164.63 mmol) was stirred at 70° C. for 2 hours. The mixture was allowed to cool to room temperature and concentrated in vacuo. The resulting solution was diluted with water (200 mL) and 6M aq. HC1 solution (-30 mL) was added causing a white precipitate to form. EtOAc (300 mL) was added and the phases were separated. The aqueous portion was further extracted with EtOAc (200 mL) and the combined organic extracts were washed with brine (200 mL), dried over MgSO₄ and concentrated in vacuo to afford the title compound as a white solid.

LC-MS (Method B): Rt 1.15 min; MS m/z 193.4=[M+H]+

1H NMR (500 MHz, Chloroform-d)δ 2.50 (d, J=7.6 Hz, 2H), 2.24 (t, J=7.5 Hz, 1H), 1.93-1.77 (m, 8H), 1.74 (d, J=11.2 Hz, 4H), 1.56 (d, J=12.5 Hz, 2H). Step 4: 2-(2-Adamantyl)-N-(4-amino-3-nitro-phenyl)acetamide

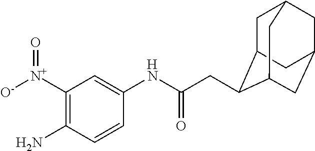

HATU (13.66 g, 35.91 mmol) was added to a cooled (0° C.) solution of 2-(2-adamantyl)acetic acid (step 3) (6.34 g, 32.65 mmol) in DMF (60 mL). DIPEA (8.53 mL, 48.97 mmol) was added dropwise over 1 min and the resulting solution stirred at 0° C. for 5 mins and at room temperature for 10 mins. The solution was cooled back to 0° C. and 2-nitrobenzene-1,4-diamine (5.0 g, 32.65 mmol) was added. The resulting solution was stirred at 0° C. for 1 hour and after warming to room temperature, diluted with water (60 mL). EtOAc (100 mL) and more water (40 mL) were added and the layers separated. The aqueous layer was extracted with EtOAc (100 mL) and the combined organic extracts were washed with saturated aqueous sodium bicarbonate solution (2×100 mL), 10% potassium carbonate solution (2×100 mL) and filtered under vacuum. The biphasic filtrate was placed into a separating funnel and the layers were separated. The organic layer was passed through a phase separating Isolute® cartridge and concentrated in vacuo to afford a dark black/brown/red gum. DCM (-80 mL) was added and the suspension was agitated. More DCM was added and the suspension was filtered under vacuum, washing with DCM and drying under vacuum to afford the title compound as a red/brown solid.

LC-MS (Method B): Rt 1.22 min; MS m/z 330.2=[M+H]+

1H NMR (500 MHz, DMSO-d6) δ 9.88 (s, 1H), 8.40 (d, J=2.5 Hz, 1H), 7.52 (dd, J=9.1, 2.5 Hz, 1H), 7.31 (s, 2H), 7.03-6.90 (m, 1H), 2.41 (d,J=7.6 Hz, 2H), 2.21 (t, J=7.5 Hz, 1H), 1.95-1.60 (m, 12H), 1.56-1.46 (m, 2H).

Step 5: 2-(2-Adamantyl)-N-(3,4-diaminophenyl)acetamide

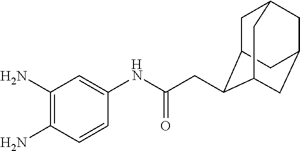

A solution of 2-(2-adamantyl)-N-(4-amino-3-nitro-phenyl)acetamide (step 4) (4.0 g, 12.14 mmol) in EtOH (60 mL) was purged with nitrogen and treated with Pd/C (10%, 1.03 g, 0.97 mmol). The mixture was placed under a hydrogen atmosphere and stirred at room temperature overnight. The resulting mixture was filtered through Celite® (filter material), washing with EtOAc, and concentrated in vacuo to afford the title compound as a brown foam.

LC-MS (Method B): Rt 0.97 min; MS m/z 300.2=[M+H]+(100% @ 215 nm)

1H NMR (500 MHz, DMSO-d6) δ 9.27 (s, 1H), 6.81 (d, J=2.3 Hz, 1H), 6.53 (dd, J=2.3, 6.5 Hz, 1H), 6.38 (d, J=6.4 Hz, 1H), 4.60-4.07 (m, 4H), 2.33 (d, J=7.6 Hz, 2H), 2.20-2.13 (m, 1H), 1.95-1.62 (m, 12H), 1.55-1.42 (m, 2H).

### Intermediate C

### 2-(1-Adamantyl)-N-(3,4-diaminophenyl)acetamide

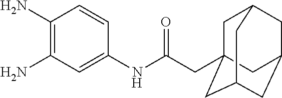

Step 1: 2-(1-Adamantyl)-N-(4-amino-3-nitro-phenyl)acetamide

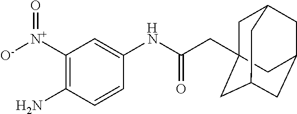

2-Nitrobenzene-1,4-diamine (3.15 g, 20.59 mmol) was added to a solution of 2-(1-adamantyl)acetic acid (4.0 g, 20.59 mmol), HATU (8.61 g, 22.65 mmol) and DIPEA (5.38 mL, 30.88 mmol) in DMF (20 mL). After stirring at room temperature for 18 hours, the reaction mixture was partitioned between EtOAc (100 mL) and water (100 mL). A black precipitate formed in the biphasic mixture. The solid was filtered off and was discarded. The phases were separated and the organic layer was washed with water (100 mL) and brine (2×50 mL), dried over MgSO₄ and was concentrated in vacuo to afford a brown/black oil. The oil was triturated in DCM (-40 mL) and the resulting suspension filtered to afford the title compound as a red/black solid.

LC-MS (Method B): Rt 1.24 min; MS m/z 330.2=[M+H]+

1H NMR (500 MHz, DMSO-d6) δ 9.73 (s, 1H), 8.39 (d, J=2.5 Hz, 1H), 7.50 (dd, J=9.1, 2.5 Hz, 1H), 7.29 (s, 2H), 6.96 (d, J=9.1 Hz, 1H), 2.00 (s, 2H), 1.93 (s, 3H), 1.69-1.63 (m, 3H), 1.63-1.54 (m, 9H).

Step 2: 2-(1-Adamantyl)-N-(3,4-diaminophenyl)acetamide

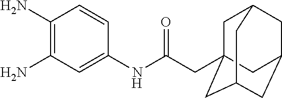

A suspension of 2-(1-adamantyl)-N-(4-amino-3-nitro-phenyl)acetamide (step 1) (4.82 g, 14.63 mmol) and Pd/C (10%, 1.24 g, 1.17 mmol) in EtOH (50 mL) was placed under a hydrogen atmosphere and stirred for 18 hours. The resulting mixture was filtered through Celite® (filter material) and the solid washed with EtOH (3×10 mL). The filtrate was concentrated in vacuo to afford the title compound as a purple solid.

LC-MS (Method B): Rt 0.93 min; MS m/z 300.3=[M+H]+

1H NMR (500 MHz, DMSO-d6) δ 9.12 (s, 1H), 6.83 (d, J=2.3 Hz, 1H), 6.52 (dd, J=8.2, 2.3 Hz, 1H), 6.38 (d, J=8.2 Hz, 1H), 4.54-4.22 (m, 4H), 1.96-1.88 (m, 5H), 1.70-1.54 (m, 12H).

### Intermediate D

### 2-(2,3-Dihydrobenzofuran-3-y0-1H-benzimidazole-5-carboxylic acid

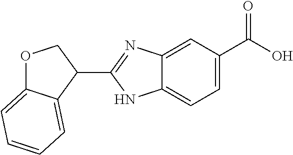 [SMILES: O=C(O)c1ccc2[nH]c(C3COc4ccccc43)nc2c1]

Step 1: Methyl 2-(2,3-dihydrobenzofuran-3-yl)-1H-benzimidazole-5-carboxylate

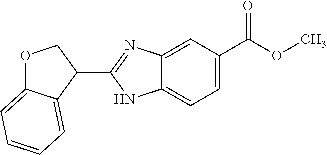 [SMILES: COC(=O)c1ccc2[nH]c(C3COc4ccccc43)nc2c1]

To a solution of 2,3-dihydrobenzofuran-3-carboxylic acid (50 mg, 0.30 mmol) in DMF (2 mL) was added DIPEA (59 μL, 0.34 mmol) and HATU (127 mg, 0.34 mmol) followed by methyl 3,4-diaminobenzoate (56 mg, 0.34 mmol) and the mixture was stirred at room temperature for 16 h. The resulting mixture was diluted with water (5 mL) and extracted with EtOAc (2×5 mL). The combined organic extracts were washed with brine (5 mL) and concentrated in vacuo. The crude residue was dissolved in acetic acid (2 mL) and heated at 60° C. for 3 h. The resulting mixture was diluted with EtOAc (10 mL) and washed with sat. NaHCO₃ solution (2×10 mL), dried over Na₂SO₄ and concentrated in vacuo. The crude residue was purified by chromatography on silica eluting with 0-100% EtOAc in heptanes to afford the title compound as a pale orange glass.

LC-MS (Method E): Rt 0.97 min; MS m/z 294.9=[M+H]+

1H NMR (500 MHz, Methanol-d4) δ 8.22 (br s, 1H), 7.91 (d, J=8.5 Hz, 1H), 7.55 (br s, 1H), 7.23-7.14 (m, 2H), 6.92-6.84 (m, 2H), 5.09-5.03 (m, 1H), 4.96 (t, J=9.4 Hz, 1H), 4.87-4.83 (obscured m, 1H), 3.91 (s, 3H).

Step 2: 2-(2,3-Dihydrobenzofuran-3-yl)-1H-benzimidazole-5-carboxylic acid To a solution of methyl 2-(2,3-dihydrobenzofuran-3-yl)-1H-benzimidazole-5-carboxylate (step 1) (90%, 69 mg, 0.21 mmol) in MeOH (0.3 mL), THF (0.3 mL) and water (0.3 mL) was added LiOH (5.6 mg, 0.23 mmol) and the mixture was stirred at room temperature for 2 h. Further LiOH (5.6 mg, 0.23 mmol.) was added and the mixture was heated to 50° C. overnight. The resulting mixture was cooled to room temperature and acidified to pH 4 using 1M HC1. The mixture was diluted with water and extracted with chloroform/IPA (2:1). The combined organic extracts were passed through a hydrophobic frit and concentrated in vacuo to afford the title compound.

LC-MS (Method E): Rt 0.84 min; MS m/z 280.9=[M+H]+

### Biological Example

### Automated whole-cell patch clamp assay to detect TMEM16A activity in recombinant cells Cell culture and preparation

Fisher rat thyroid (FRT) cells stably expressing human TMEM16A (TMEM16Aabc variant; Dr Luis Galietta, Insituto Giannina, Italy) were cultured in T-75 flasks in Hams F-12 media with Coon's modification (Sigma) supplemented with 10% (v/v) foetal bovine serum, penicillin-streptomycin (10,000 U/mL/10000 μg/mL), G-418 (750μg/mL), L-glutamine (2 mM) and sodium bicarbonate solution (7.5% v/v). At —90% confluence cells were harvested for experiments by detachment with a 2:1 (v/v) mixture of Detachin (BMS Biotechnology) and 0.25% (w/v) trypsin-EDTA. Cells were diluted to a density of 3.5-4.5×10 6 cells/mL with media consisting of CHO-S-SFM II (Sigma), 25 mM HEPES (Sigma) and Soy bean trypsin inhibitor (Sigma).

### Whole-Cell Patch Clamp Recording

FRT-TMEM16A cells were whole-cell patch clamped using an automated planar patch clamp system (Qpatch, Sophion). Briefly, once high resistance (GOhm) seals were established between the cells and the planar recording array the patch was ruptured using suction pulses to establish the whole-cell recording configuration of the patch clamp technique. The assay employed the following solutions (all reagents Sigma):

Intracellular solution (mM): N-methyl-D-glucamine 130, CaCl₂ 18.2, MgCl₂ 1, HEPES 10, EGTA 10, BAPTA 20, Mg-ATP 2, pH 7.25, 325mOsm with sucrose.

Extracellular solution (mM): N-methyl-D-glucamine 130, CaCl₂ 2, MgCl₂ 1, HEPES 10, pH 7.3, 320 mOsm with sucrose.

The intracellular solution buffers intracellular calcium at levels required to give —20% activation of the maximal TMEM16A mediated current (EC20 for calcium ions). Cells were voltage clamped at a holding potential of -70mV and a combined voltage step (to +70 mV)/ramp (−90 my to +90 mV) was applied at 0.05 Hz. After a period of current stabilisation test compounds, solubilised in 100% (v/v) DMSO and subsequently diluted into extracellular solution, were applied to generate a cumulative concentration response curve. Each concentration of test compound was incubated for 5 minutes before addition of the next concentration. After the final concentration was tested a supramaximal concentration of either a known active positive modulator or the TMEM16A inhibitor, CaCCinhA01 (Del La Fuente et al, 2008) was added to define the upper and lower limits of the assay.

Compound activity was quantified by measuring the increase in current upon compound addition and expressing this as a percentage increase of baseline TMEM16A current level. Percentage increases in current were determined for each concentration and the data plotted as a function of concentration using either the Qpatch software or Graphpad Prism v6.05 providing the concentration which gave 50% of its maximal effect (EC₅₀) and maximum efficacy (percentage of baseline increase).

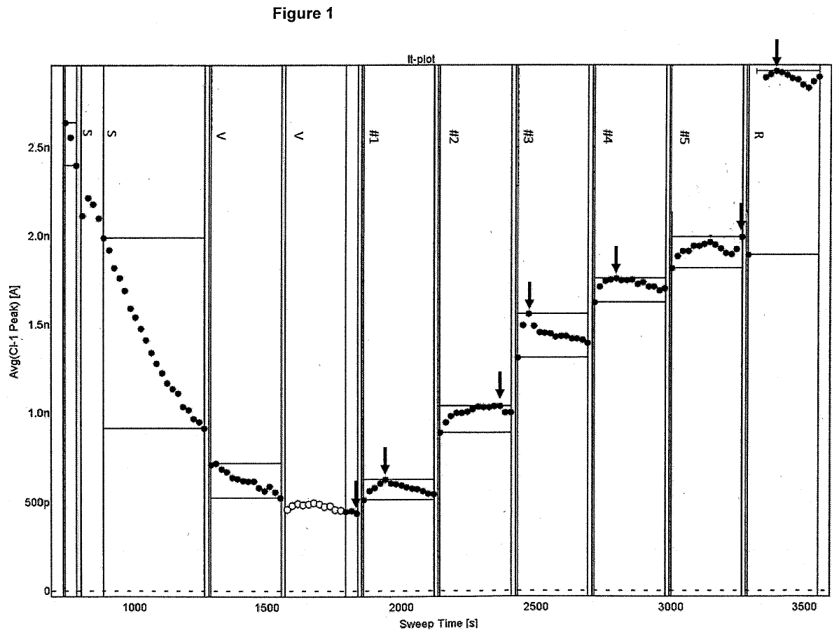

The method of calculating the results is illustrated in FIG. 1, which shows an example trace from the Qpatch TMEM16A assay. In FIG. 1, IBL equals baseline current, km equals the peak current during test compound concentration 1 incubation period and so on.

Peak TMEM16A current at +70mV was plotted as a function of time over the assay period. Baseline current (I_{BL}) was measured after a period of stabilisation. The increase in current for each compound addition was determined by taking the peak current during the incubation period and subtracting the current from the previous recording period and then expressing this as a percentage of the baseline current. For test compound concentration 1 in FIG. 1 this is:

(I_{[#1]}−I_{BL}/I_{BL})×100

For each additional concentration tested the increase in current was determined by subtracting the current from the previous incubation period and normalising the baseline value—for test concentration 2 in FIG. 1 this is:

(I_{[#2]}−I_{[#1]}/I_{BL})×100

The values for each test concentration were plotted as a cumulative function of concentration e.g. for test concentration two this would be the sum of the peak changes measured during concentration one plus concentration two.

The results for % potentiation at 3.33μM obtained for the example compounds are shown in Table 2, from which it can be seen that the compounds of the present invention are capable of significantly increasing the TMEM16A current level.

TABLE 2
% Potentiation shown by 3.33 μM solution
of Test Compounds and Calculated EC₅₀ Values

| | % Potentiation | |
|---|---|---|
| Example | @ 3.33 μM Avg | EC₅₀ Avg (μM) |
| 1.1 | 127.42 | 0.250 |
| 1.2 | 111.46 | 0.225 |
| 1.2.1 | 145.99 | 0.441 |
| 1.2.2 | 144.25 | 0.203 |
| 1.2.3 | 153.06 | 0.792 |
| 1.3 | 78.45 | 0.563 |
| 1.3.1 | 134.61 | 0.425 |
| 1.3.2 | 92.92 | 0.581 |
| 1.4 | 81.29 | 0.384 |
| 1.5 | 162.43 | 1.084 |
| 1.5.1 | 105.43 | 0.222 |
| 1.5.2 | 135.94 | 0.227 |
| 1.5.3 | 93.11 | 0.566 |
| 1.5.4 | 218.70 | 0.834 |
| 1.6 | 83.87 | 0.231 |
| 1.7 | 136.32 | 0.587 |
| 1.7.1 | 156.93 | 0.273 |
| 1.7.2 | 143.30 | 0.294 |
| 1.7.3 | 114.41 | 1.12 |
| 1.7.4 | 148.28 | 1.31 |
| 1.8 | 126.24 | 0.260 |
| 1.9 | 52.097 | — |
| 1.10 | 110.07 | 0.892 |
| 1.10.1 | 124.47 | 0.210 |
| 1.10.2 | 76.062 | 0.192 |
| 2.1 | 214.76 | 0.860 |
| 2.1.1 | 47.606 | 0.582 |
| 2.2 | 197.3 | 0.622 |
| 2.2.1 | 97.67 | 0.907 |
| 2.2.2 | 127.18 | 0.948 |
| 2.2.3 | 154.64 | 1.035 |
| 2.2.4 | 190.41 | 1.079 |
| 2.2.5 | 112.54 | 0.758 |
| 2.2.6 | 167.14 | 0.780 |
| 2.3 | 200.58 | 1.069 |
| 2.4 | 133.35 | 0.658 |
| 2.5 | 58.86 | 0.150 |
| 3.1 | 74.05 | 0.287 |
| 3.2 | 58.487 | 0.332 |
| 3.3 | 113.72 | 0.554 |
| 3.3.1 | 95.75 | 0.471 |
| 3.3.2 | 90.25 | 0.598 |
| 3.4 | 99.79 | 0.471 |
| 3.4.1 | 116.41 | 0.576 |
| 3.5 | 143.57 | 1.018 |
| 3.5.1 | 89.54 | 0.779 |

### Sheep Mucociliary Clearance (MCC)

MCC in sheep was measured as described by Coote et al., “NVP-QBE170: an inhaled blocker of the epithelial sodium channel with a reduced potential to induce hyperkalaemia,” Br J Pharmacol. 2015 Jun; 172(11): 2814-2826. Briefly, adult ewes are nasally intubated with test compounds delivered as dry powder lactose blends. Hypertonic saline and water control are administered to the sheep by nebulization via endotracheal tube. Aerosolized technetium labelled sulphur colloid (⁹⁹ mTc-SC) is used to measure the effects of the various doses of test compounds or control on MCC by gamma scintigraphy. The ewes are administered ⁹⁹ mTc-SC at selected time intervals following administration of test substances. Serial images are taken periodically and counts from the right lung are corrected for decay and expressed as a percentage of radioactivity cleared relative to the baseline image (% cleared). Differences in clearance of ⁹⁹ mTc-SC are compared at time intervals after radioaerosol administration. See also Hirsh et al., ‘Pharmacological properties of N-(3,5-diamino-6-chloropyrazine-2-carbonyl)-N’-444-(2,3-dihydroxypropoxy)phenyllbutyl-guanidine methanesulfonate, a novel epithelial sodium channel blocker with potential clinical efficacy for cystic fibrosis lung disease,” J Pharmacol Exp Ther. 2008 Apr; 325(1):77-88, and Coote et al., “Camostat attenuates airway epithelial sodium channel function in vivo through the inhibition of a channel-activating protease,” J Pharmacol Exp Ther. 2009 May; 329(2):764-74.

All literature and patent documents referred to herein are incorporated by reference to the fullest extent possible.

### REFERENCES

Accurso F J, Moss R B, Wilmott R W, Anbar R D, Schaberg A E, Durham T A, Ramsay B W; TIGER-1 Investigator Study Group (2011) Denufosol tetrasodium in patients with cystic fibrosis and normal to mildly impaired lung function. Am J Respir Crit Care Med, 183(5):627-634.
Boucher RC (2007) Evidence for airway surface dehydration as the initiating event in C F airway disease. J Intern Med., 261(1):5-16.
Caputo A, Caci E, Ferrera L, Pedemonte N, Barsanti C, Sondo E, Pfeffer U, Ravazzolo R, Zegarra-Moran O & Galietta L J (2008) TMEM16A, a membrane protein associated with calcium-dependent chloride channel activity. Science, 322(5901):590-594.
Del La Fuente R, Namkung W, Mills A & Verkman A S (2008) Small molecule screen identifies inhibitors of a human intestinal calcium-activated chloride channel Mol Pharmacol, 73(3):758-768.
Kellerman D, Rossi Mospan A, Engels J, Schaberg A, Gorden J & Smiley L (2008) Denufosol: a review of studies with inhaled P2Y(2) agonists that led to Phase 2. Pulm Pharmacol Ther, 21(4):600-607.
Kunzelmann K & Mall M (2003) Pharmacotherapy of the ion transport defect in cystic fibrosis: role of purinergic receptor agonists and other potential therapeutics. Am J Respir Med, 2(4):299-309.
Matsui H, Grubb B R, Tarran R, Randell S H, Gatzy J T, Davis C W and Boucher R C (1998) Evidence for periciliary liquid layer depletion, not abnormal ion composition, in the pathogenesis of cystic fibrosis airways disease. Cell, 95(7):1005-15.
Moss R B (2013) Pitfalls of drug development: lessons learned from trials of denufosol in cystic fibrosis. J Pediatr, 162(4):676-680.
Pedemonte N & Galietta L J (2014) Structure and function of TMEM16 proteins (anoctamins). Physiol Rev, 94(2):419-459.
Pezzulo A A, Tang X X, Hoegger M J, Abou Alaiwa M H, Ramachandran S, Moninger T O, Karp P H, Wohlford-Lenan C L, Haagsman H P, van Eijk M, Banfi B, Horswill A R, Stoltz D A, McCray P B Jr, Welsh MJ & Zabner J (2012) redcued airway surface pH impairs bacterial killing in the porcine cystic fibrosis lung. Nature, 487(7405):109-113.
Yang Y D, Cho H, Koo J Y, Tak M H, Cho Y, Shim W S, Park SP, Lee J, Lee B, Kim B M, Raouf R, Shin Y K & Oh U (2008) TMEM16 confers receptor-activated calcium-dependent chloride conductance. Nature, 455(7217):1210-1215.

1. A compound of general formula (I): 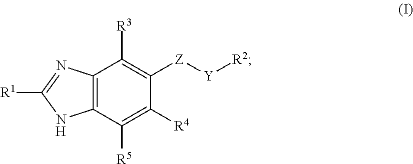
or a stereoisomer, solvate, or pharmaceutically acceptable salt thereof,
wherein:
R¹ is
i. [CH(R⁷)]ₙ—N(R⁸—C(O)OR⁹;
n is 1 or 2;
each R⁷ is independently H, phenyl or C₁₋₃ alkyl optionally substituted with one or more substituents selected from OH and OCH_{3;}
R⁸ is H, C₁₋₃ alkyl optionally substituted with one or more substituents selected from OH and methoxy;
R⁹ is C₂₋₄ alkyl; or
ii. CH(R¹¹)(R¹²);
R¹¹ is H, OH, CH₃, CH₂OH or a group which combines with a substituent on R¹² as defined below;
R¹² is selected from cyclohexyl optionally substituted with one or more substituents selected from OH and methoxy; and phenyl or 5- or 6-membered heteroaryl, wherein said phenyl or heteroaryl is optionally substituted with one or more substituents selected from OH, methoxy, methyl, fluoro, chloro and a substituent which, together with R¹¹ and the atoms to which it is attached, forms a 5- or 6-membered oxygen-containing heterocyclic ring fused to the phenyl or heteroaryl group R¹²; or
iii. C₂₋₆ alkyl optionally substituted with OR¹⁵, wherein R′5 is methyl or ethyl; or
iv. 6- to 10-membered aryl or 5- to 10-membered heteroaryl, either or which is optionally substituted with one or more substituents selected from fluoro, chloro, OH or methoxy;
Z is selected from —NH—C(O)— and —C(O)—NH—;
Y is selected from —CH₂— and —CH(CH₃)—; or Y combines with R² as defined below; and
R² is selected from:
i. a 3- to 10-membered carbocyclic ring system or a 6- to 10-membered aryl or 5- to 10-membered heteroaryl ring system, wherein the aryl, heteroaryl or carbocyclic ring system is optionally substituted with one or more substituents selected from fluoro; chloro; CN; nitro; OH; C₁₋₆ alkyl optionally substituted with one or more substituents selected from halo, OH and CN; O(C₁₋₆ alkyl) optionally substituted with one or more substituents selected from halo, OH and CN; and
CH₂NH—C(O)O—C₁₋₆ alkyl optionally substituted with one or more substituents selected from halo and OH; or
Y and R² together form an unsubstituted C₃₋₈ alkyl group or a group CH₂—C(R¹⁷)(R¹⁸)—CH₂—N(R¹⁹)R20;
wherein each of R¹⁷, R¹⁸ and R¹⁹ is independently H or C₁₋₄ alkyl; and
R²⁰ is C₁₋₄ alkyl or C₁₋₄ haloalkyl;
R³, R⁴ and R⁵ are each independently either H or F;
provided that:
A. when R¹ is CH(R¹¹)(R¹²) ; where R¹¹ is H or methyl and R¹² is phenyl which is unsubstituted or substituted with 1 or 2 substituents, wherein the substituents are selected from halo and methoxy:
R² is not phenyl or heteroaryl, wherein said phenyl or heteroaryl is optionally substituted with 1 or 2 substituents selected from halo, C₁₋₄ alkyl, C₁₋₄ alkoxy and a 5-membered heteroaryl ring; and
B. when R¹ is CH(R¹¹)(R¹²); where R¹² is phenyl and R¹¹ together with a substituent on R¹² and the atoms to which they are attached combine to form a 5- or 6-membered ring fused to the phenyl ring R¹², wherein the 5- or 6-membered ring is optionally substituted with C₁₋₃ alkyl:
i. R² is not phenyl or heteroaryl, wherein said phenyl or heteroaryl is optionally substituted with 1, 2 or 3 substituents, wherein the substituents are selected from halo, C₁₋₄ alkyl, C₁₋₄ haloalkyl and C₁₋₄ alkoxy; and
ii. Y and R² do not combine to form C₃₋₁₀ alkyl; and
C. when R¹ is CH(R¹¹)(R¹²); where R¹¹ is H and R¹² is cyclohexyl:
R² is not phenyl optionally substituted with 1, 2 or 3 substituents, wherein the substituents are selected from halo, methyl, methoxy; unsubstituted 5- to 8 membered heteroaryl.

2. The compound according to claim 1, wherein R² is unsubstituted cyclohexyl and R¹ is CH(R¹¹)(R¹²), where R¹¹ and R¹² are as defined in claim 1.

3. The compound according to claim 2, wherein R¹² is phenyl optionally substituted with OH or methoxy.

4. The compound according to claim 3, wherein R¹ is unsubstituted benzyl and R² is unsubstituted cyclohexyl.

5. The compound according to claim 4 selected from:
N-(2-B enzyl-1H-b enzimi dazol-5-yl)-2-cycl ohexyl -acetami de;
2-Benzyl-N-(cyclohexylmethyl)-1H-benzimidazole-5-carboxamide; and
salts and solvates thereof.

6-8. (canceled)

9. The compound of claim 1:
selected from:
N-(2-Benzyl-1H-benzimidazol-5-yl)-2-cyclohexyl-acetamide (Compound 1.1);
2-Benzyl-N-(cyclohexylmethyl)-1H-benzimidazole-5-carboxamide (Compound 1.2);
N-(1-Adamantylmethyl)-2-benzyl-1H-benzimidazole-5-carboxamide (Compound 1.2.1);
2-Benzyl-N- [(1-methylcyclopentyl)methyl]-1 H-benzimidazole-5-carboxamide (Compound 1.2.2);
2-Benzyl-N-[(1R)-1-cyclohexylethyl]-1H-benzimidazole-5-carboxamide (Compound 1.2.3);
N-(Cycloheptylmethyl)-2-(1,1-dimethylpropyl)-3H-benzimidazole-5-carboxamide Compound 1.3);
N-(Cycloheptylmethyl)-2-[(1-hydroxycyclohexyl)methyl]-1H-benzimidazole-5-carboxamide (Compound 1.3.1);
N-(Cycloheptylmethyl)-2-(2-hydroxy-1-phenyl-ethyl)-1H-benzimidazole-5-carboxamide (Compound 1.3.2);
N-(Cyclohexylmethyl)-2-[(3-hydroxyphenyl)methyl]-3H-benzimidazole-5-carboxamide (Compound 1.4);
2-(1-Adamantyl)-N-[2-[(3-hydroxyphenyl)methyl]-1H-benzimidazol-5-yl]acetamide (Compound 1.5);
N[2-[(3-hydroxyphenyl)methyl]-1H-benzimidazol-5-yl]-2-(1-methylcyclohexyl) acetamide (Compound 1.5.1);
2-Cycloheptyl-N[2-[(3-hydroxyphenyl)methyl]-1H-benzimidazol-5-yl]acetamide (Compound 1.5.2);
2-Cyclohexyl-N[2-[(3-hydroxyphenyl)methyl]-1H-benzimidazol-5-yl]acetamide (Compound 1.5.3);
2-(1-Adamantyl)-N-(2-benzyl-1H-benzimidazol-5-yl)acetamide (Compound 1.5.4);
N-(2-Benzyl-1H-benzimidazol-5-yl)-2-(2-hydroxy-2-adamantyl)acetamide (Compound 1.6);
2-(2-Adamantyl)-N[2-[(3-methoxyphenyl)methyl]-1H-benzimidazol-5-yl]acetamide (Compound 1.7);
2-(2-Adamantyl)-N[2-[(1s)-1-phenylethyl]-1H-benzimidazol-5-yl]acetamide (Compound 1.7.1);
2-(2-Adamantyl)-N[2-[(1R)-1-phenylethyl]-1H-benzimidazol-5-yl]acetamide (Compound 1.7.2);
tert-Butyl N-[[5-[[2-(1-adamantyl)acetyl]amino]-1H-benzimidazol-2-yl]methyl]carbamate (Compound 1.7.3);
2-(1-Adamantyl)-N-[2-[(2-methoxy-3-pyridyl)methyl]-1H-benzimidazol-5-yl]acetamide (Compound 1.7.4);
2-(2-Adamantyl)-N[2-[(3-hydroxyphenyl)methyl]-1H-benzimidazol-5-yl]acetamide (Compound 1.8);
2-tert-Butyl-N-[(5-chloro-2-hydroxy-phenyl)methyl]-1H-benzimidazole-5-carboxamide (Compound 1.9);
tert-Butyl N[[5-(cycloheptylmethylcarbamoyl)-1H-benzimidazol-2-yl]methyl] carbamate (Compound 1.10);
2-Benzyl-N-[(1-methylcyclohexyl)methyl]-1H-benzimidazole-5-carboxamide (Compound 1.10.1);
2-Benzyl-N-(cyclooctylmethyl)-1H-benzimidazole-5-carboxamide (Compound 1.10.2);
tert-Butyl N-[[1-[2-[(2-benzyl-1H-benzimidazol-5-yl)amino]-2-oxo-ethyl]cyclohexyl] methyl]carbamate (Compound 2.1);
N-(2-Benzyl-1H-benzimidazol-5-yl)-2-(4,4-difluorocyclohexyl)acetamide (Compound 2.1.1);
tert-Butyl N-[1-[54[2-(2-adamantyl)acetyl]amino]-1H-benzimidazol-2-yl]-2-methoxy-ethyl]carbamate (Compound 2.2);
tert-Butyl N-[(R)45-[[2-(2-adamantyl)acetyl]amino]-1H-benzimidazol-2-yl]-phenyl-methyl]-N-methyl-carbamate Compound 2.2.1;
tert-Butyl N-RS)45-[[2-(2-adamantyl)acetyl]amino]-1H-benzimidazol-2-yl]-phenyl-methyl]-N-methyl-carbamate (Compound 2.2.2);
tert-Butyl N-[[64[2-(2-adamantyl)acetyl]amino]-1H-benzimidazol-2-yl]methyl]-N-ethyl-carbamate (Compound 2.2.3);
tert-Butyl N-[[5-[[2-(2-adamantyl)acetyl]amino]-1H-benzimidazol-2-yl]methyl]-N-(2-methoxyethyl)carbamate (Compound 2.2.4);
2-(2-Adamantyl)-N[2-(2-methoxyethyl)-3H-benzimidazol-5-yl]acetamide (Compound 2.2.5);
2-(2-Adamantyl)-N[2-(3-methoxypropyl)-1H-benzimidazol-5-yl]acetamide (Compound 2.2.6);
tert-Butyl N-[[5-[[2-(2-adamantyl)acetyl]amino]-1H-benzimidazol-2-yl]methyl]-N-methyl-carbamate (Compound 2.3);
N-(Cycl oheptylmethyl)-2-(2,3-dihydrob enzofuran-3-yl)-1H-b enzimi dazol e-5-carb oxami de (Compound 2.4);
2-(2-adamantyl)-N[2-[hydroxy(phenyl)methyl]-1H-benzimidazol-5-yl]acetamide (Compound 2.5);
2-Cyclohexyl-N-(2-phenyl-1H-benzimidazol-5-ypacetamide (Compound 3.1);
N-(2-Benzyl-1H-benzimidazol-5-yl)adamantane-1-carboxamide (Compound 3.2);
N-(Cycloheptylmethyl)-7-fluoro-2-[(3-hydroxyphenyl)methyl]-1H-benzimidazole-5-carboxamide (Compound 3.3);
N-(Cycloheptylmethyl)-6-fluoro-2-[(3-hydroxyphenyl)methyl]-1H-benzimidazole-5-carboxamide (Compound 3.3.1);
N-(Cycloheptylmethyl)-4-fluoro-2-[(3-hydroxyphenyl)methyl]-1H-benzimidazole-5-carboxamide (Compound 3.3.2);
tert-Butyl N-[245-[[2-(2-adamantyl)acetyl]amino]-1H-benzimidazol-2-yflethyl]carbamate (Compound 3.4);
2-(2-Adamantyl)-N[2-[(3,5-dimethylisoxazol-4-yl)methyl]-1H-benzimidazol-5-yl]acetamide (Compound 3.4.1);
2-Benzyl-N-(2,2-dimethylpropyl)-1H-benzimidazole-5-carboxamide (Compound 3.5);
2-Benzyl-N-(1,1,2,2-tetramethylpropyl)-1H-benzimidazole-5-carboxamide (Compound 3.5.1); or a stereoisomer, solvate, or pharmaceutically acceptable salt thereof.

10. A compound of general formula (IA): 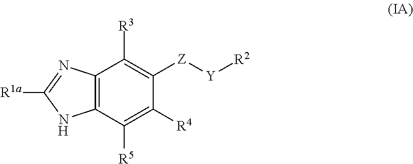
wherein R², R³, R⁴, R⁵, Y and Z are as defined for general formula (I) in claim 1 and: R^{1a} is
i. [CH(R^{7a})]ₙ—N(R^{8a})—C(O)OR^{9a};
n is 1 or 2;
each R⁷ is independently H, phenyl or C₁₋₃ alkyl optionally substituted with one or more substituents selected from OH and OCH₃;
R⁸ is H, C₁₋₃ alkyl optionally substituted with one or more substituents selected from OH and methoxy;
provided that when n is 1, R^{7a} and R^{8a} are not both H;
R^{9a} is C₂₋₄ alkyl; or
ii. CH(R^{11a})(R^{12a});
R^{11a} is H, OH, CH₃, CH₂OH or a group which combines with a substituent on R^{12a} as defined below;
R^{12a} is selected from phenyl or 5- or 6-membered heteroaryl, wherein said phenyl or heteroaryl is optionally substituted with one or more substituents selected from OH, methoxy, methyl, fluoro, chloro and a substituent which, together with R^{11a} and the atoms to which it is attached, forms a 5- or 6-membered oxygen-containing heterocyclic ring fused to the phenyl or heteroaryl group R^{12a};
provided that when R^{12a} is phenyl or 6-membered heteroaryl optionally substituted with one or more substituents selected from OH, methoxy, methyl, fluoro or chloro, R^{11a} is not H; or
iii. methyl or ethyl substituted with OR^{15a}, wherein R^{15a} is methyl or ethyl; or
iv. 6- to 10-membered aryl or 5- to 10-membered heteroaryl, either or which is optionally substituted with one or more substituents selected from fluoro, chloro, OH or methoxy; provided that:
A. when R^{1a} is CH(R₁₁ₐ)(R^{12a}) ; where R^{11a} is H or methyl and R^{12a} is phenyl which is unsubstituted or substituted with 1 or 2 substituents, wherein the substituents are selected from halo and methoxy:
i. R² is not phenyl or heteroaryl, wherein said phenyl or heteroaryl is optionally substituted with 1 or 2 substituents selected from halo, C₁₋₄ alkyl, C₁₋₄ alkoxy and a 5-membered heteroaryl ring; and
B. when R^{1a} is CH(R^{11a})(R^{12a}) ; where R^{12a} is phenyl and R^{11a} together with a substituent on R^{12a} and the atoms to which they are attached combine to form a 5- or 6-membered ring fused to the phenyl ring R^{12a}, wherein the 5- or 6-membered ring is optionally substituted with C₁₋₃ alkyl:
i. R^{2a} is not phenyl or heteroaryl, wherein said phenyl or heteroaryl is optionally substituted with 1, 2 or 3 substituents, wherein the substituents are selected from halo, C₁₋₄ alkyl, C₁₋₄ haloalkyl and C₁₋₄ alkoxy; and
ii. Y and R² do not combine to form C₃₋₁₀ alkyl;
or a stereoisomer, solvate, or pharmaceutically acceptable salt thereof.

11. The compound according to claim 10, wherein R^{1a} is [CH(R^{7a})]ₙ—N(R⁸ a—C(O)OR⁹ a, wherein n, R^{7a}, R^{8a} and R^{9a} are as defined in claim 10.

12.-16. (Canceled)

17. The compound according to claim 10, wherein R^{1a} is CH(R^{11a})(R^{12a}) , wherein R^{1a} and R^{12a} are as defined in claim 10.

18.-24. (canceled)

25. A compound of general formula (IB): 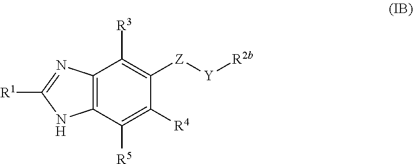
wherein R¹, R³, R⁴, R⁵, Y and Z are as defined for general formula (I) in claim 1 and:
R^{2b} is selected from:
a 3- to 10-membered carbocyclic ring system substituted with one or more substituents selected from fluoro; chloro; CN; nitro; OH; C₁₋₆ alkyl optionally substituted with one or more substituents selected from halo, OH and CN; O(C₁₋₆ alkyl) optionally substituted with one or more substituents selected from halo, OH and CN; and CH₂NH—C(O)O—C₁₋₆ alkyl optionally substituted with one or more substituents selected from halo and OH; or
Y and R^{2b} together form an unsubstituted C₃₋₈ alkyl group or a group CH₂—C(R^{17b})(R^{18b})—CH₂—N(R^{19b})R^{20b};
wherein each of R^{17b}, R^{18b} and R^{19b} is independently H or C₁₋₄ alkyl; and
R^{20b} is C₁₋₄ alkyl or C₁₋₄ haloalkyl;
provided that:
when R¹ is CH(R¹¹)(R¹²); where R¹² is phenyl and Ru together with a substituent on R¹² and the atoms to which they are attached combine to form a 5- or 6-membered ring fused to the phenyl ring R¹², wherein the 5- or 6-membered ring is optionally substituted with C₁₋₃ alkyl:
Y and R^{2b} do not combine to form C₃₋₈ alkyl
or a stereoisomer, solvate, or pharmaceutically acceptable salt thereof.

26. The compound according to claim 25, wherein R^{2b} is a 3- to 10-membered carbocyclic ring system substituted as defined in claim 25.

27. The compound according to claim 26, wherein R^{2b} is adamantyl substituted with OH.

28. The compound according to claim 26, wherein R^{2b} is a carbocyclic ring system, selected from cyclopentyl, cyclohexyl, cycloheptyl and cyclooctyl, any of which substituted with one or more substituents selected from OH, fluoro, C₁₋₆ alkyl, O(C₁₋₆ alkyl), and NH—C(O)O—C₁₋₆ alkyl, especially OH, C₁₋₄ alkyl, O(C₁₋₄ alkyl) and CH₂ NH—C(O)O—C₁₋₄ alkyl.

29. (canceled) .

30. The compound according to claim 25, wherein:
Y and R^{2b} together form an unsubstituted C₃₋₈ alkyl group; or
Y and R^{2b} together form a group CH₂—C(R^{17b})(R^{18b})—CH₂—N(R^{19b})R^{20b},
wherein each of R^{17b}, R^{18b} and R^{19b} is independently H or methyl and R^{20b} is C₁₋₄ haloalkyl.

31. A method for the treatment or prophylaxis of diseases and conditions affected by modulation of TMEM16A, the method comprising administering to a patient an effective amount of a compound according to claim 1.

32. The method according to claim 31, wherein the diseases and conditions affected by modulation of TMEM16A are selected from respiratory diseases and conditions, dry mouth (xerostomia), intestinal hypermobility, cholestasis and ocular conditions.

33. (canceled)

34. A pharmaceutical composition comprising a compound according to claim 1 and a pharmaceutically acceptable excipient.

35.-38 (canceled)
